(19) United States
(12) Patent Application Publication
(10) Pub. No.: US 2011/0263460 A1
(43) Pub. Date: Oct. 27, 2011

(54) COMPOSITIONS AND METHODS FOR PRODUCING REPLICATION COMPETENT HUMAN IMMUNODEFICIENCY VIRUS (HIV)

(75) Inventors: Miguel E. Quinones-Mateu, Rocky River, OH (US); Jan Weber, Shaker Heights, OH (US)

(73) Assignee: Diagnostic Hybrids, Inc.

(21) Appl. No.: 12/765,155

(22) Filed: Apr. 22, 2010

(51) Int. Cl. C40B 40/08; C12N 15/63; C12Q 1/70; C12N 7/01; C40B 50/02

(52) U.S. Cl. 506/17; 435/235.1; 506/24; 435/5; 435/320.1

(57) ABSTRACT

The invention provides methods for producing a replication competent chimeric human immunodeficiency virus (HIV) that optionally contains a heterologous reporter gene, and methods for generating these viruses. The invention's recombinant viruses are useful in the determination of, for example, antiretroviral drug susceptibility, HIV drug resistance, HIV phenotyping, HIV genotyping, HIV fitness, HIV tropism or coreceptor usage, HIV serum neutralization, and for HIV vaccine development, HIV vector development, and HIV virus production.

### FIELD OF INVENTION

The invention provides methods for producing a replication competent chimeric human immunodeficiency virus (HIV) that optionally contains a heterologous reporter gene, and methods for generating these viruses. The invention's recombinant viruses are useful in the determination of, for example, antiretroviral drug susceptibility, HIV drug resistance, HIV phenotyping, HIV genotyping, HIV fitness, HIV tropism or coreceptor usage, HIV serum neutralization, and for HIV vaccine development, HIV vector development, and HIV virus production.

### BACKGROUND

The research community and pharmaceutical companies have been successful in developing and testing many antiretroviral (ARV) drugs that block HIV-1W-1 replication. To date, more than 25 ARVs have been approved for therapy. However, a significant concern for HIV-infected individuals, and from a public health perspective, is the emergence of drug resistance. Once a patient starts on highly active antiretroviral therapy (HAART), emergence of ARV resistance and subsequent virological failure is almost inevitable and as a consequence, must be monitored to avoid resumption in disease and to justify new treatment alternatives. Determination of the resistance phenotype to all drugs permits an informed decision for new treatments because cross-resistance can limit the use of other drugs. Thus, monitoring drug resistance has become an important clinical tool in the management of HIV-infected patients.

What is needed are improved phenotypic and genotypic assays that provide faster and more meaningful data to determine the resistance and/or susceptibility of HIV to anti-HIV drugs, to guide treatment decisions, and manage complex anti-viral drug paradigms in order to provide an optimal treatment regimen that is individualized for each patient.

### SUMMARY OF THE INVENTION

The invention provides an in vitro method for producing a replication competent chimeric human immunodeficiency virus (HIV) that optionally contains a heterologous reporter gene, comprising a) providing 1) a first DNA sequence encoding an HIV RNA sequence, 2) a first restriction enzyme, 3) a second restriction enzyme, 4) a first yeast vector that lacks a second DNA sequence encoding HIV 5′ long terminal repeat (LTR), and that comprises a third DNA sequence encoding an HIV genome sequence, wherein the HIV genome sequence contains, in place of a sequence that corresponds to the first DNA sequence, i) a restriction sequence which can be specifically cleaved by the first restriction enzyme, and ii) a restriction sequence which can be specifically cleaved by the second restriction enzyme, 5) a second vector that comprises, in operable combination, i) a fourth DNA sequence encoding an HIV genome sequence, wherein the HIV genome sequence comprises a heterologous sequence in place of the sequence corresponding to the first DNA sequence, and wherein the heterologous sequence is flanked by A) a restriction sequence which can be specifically cleaved by the first restriction enzyme, and B) a restriction sequence which can be specifically cleaved by the second restriction enzyme, and ii) optionally a heterologous reporter gene, and 6) a host cell, b) introducing the first DNA sequence by homologous recombination into the first yeast vector to produce a second yeast vector that comprises the first DNA sequence flanked by i) a restriction sequence which can be specifically cleaved by the first restriction enzyme, and ii) a restriction sequence which can be specifically cleaved by the second restriction enzyme, c) contacting the second yeast vector produced in step b) with the first restriction enzyme and with the second restriction enzyme, wherein the contacting produces a cleaved nucleotide sequence comprising the first DNA sequence, d) introducing the cleaved nucleotide sequence produced in step c) into the second vector under conditions to substitute the heterologous sequence with the first DNA sequence, thereby producing a fourth vector that comprises, in operable combination, i) a fifth DNA sequence encoding an HIV genome sequence, wherein the HIV genome comprises the first DNA sequence in place of the sequence corresponding to the first DNA sequence, and ii) the optional heterologous reporter gene, and e) transfecting the fourth vector into the host cell to produce a replication competent chimeric HIV that comprises the first DNA sequence operably linked to the optional heterologous reporter gene. In one embodiment, the method comprises, prior to the transfecting of step e), transforming the fourth vector into a bacterial cell to produce a transformed bacterial cell. In an alternative embodiment, the method further comprises purifying the fourth vector from the transformed bacterial cell. In another alternative embodiment, the method further comprises f) contacting the replication competent chimeric HIV produced by step e) with a test compound. In yet another embodiment, the method further comprises g) determining phenotypic susceptibility of the HIV, that is produced in step e), to the test compound. In a further embodiment, the method further comprises h) generating a database that comprises the phenotypic susceptibility of the HIV, that is produced by step e), to the test compound. In yet another embodiment of the invention's methods, the HIV RNA sequence comprises at least one mutation relative to a reference HIV RNA sequence, and wherein the database comprises a listing of the mutation. In a further embodiment of the method, the steps from step a) to step d) do not include propagation of an HIV particle, that comprises the first DNA sequence, by a producer cell. In another embodiment, the heterologous sequence of step a)5) is selected from the group of a linker sequence and a lethal gene sequence. In a further embodiment, the first DNA sequence that is comprised in the replication competent chimeric HIV produced by step e), has 100% identity to the first DNA sequence in step a)1). In yet another embodiment, the replication competent chimeric HIV that is produced by step e) is infectious of a cell that is susceptible to HIV. In another embodiment, the HIV RNA sequence of step a)1) is from a sample obtained from an HIV-infected subject. In one embodiment, the first DNA sequence is produced by reverse-transcribing and amplifying the HIV RNA sequence. In another embodiment, the first yeast vector further comprises a heterologous reporter gene. In an alternative embodiment, the first yeast vector of step a)4) comprises pRECnfl-TRPΔ(p2-INT)/URA3-hRluc having SEQ ID NO:08. In a particular embodiment, the second vector of step 5) comprises pNL4-3-Δ(p24-VPR)-hRluc having SEQ ID NO:07.

The invention also provides a composition comprising a replication competent chimeric HIV, expressing an optional heterologous reporter gene, produced by any of the methods described herein.

The invention further provides a database produced by any of the methods described herein.

Also provided by the invention is a composition comprising a vector that a) lacks a DNA sequence encoding HIV 5′ long terminal repeat (LTR), and b) comprises an HIV genome sequence that contains, in place of a first DNA sequence encoding an HIV RNA sequence, i) a restriction sequence which can be specifically cleaved by a first restriction enzyme, and ii) a restriction sequence which can be specifically cleaved by a second restriction enzyme. In one embodiment, the vector further comprises a reporter gene. In a further embodiment, the vector comprises pRECnfl-TRPΔ(p2-INT)/URA3-hRluc having SEQ ID NO:08. In yet another embodiment, the vector further comprises a second DNA sequence that corresponds to the first DNA sequence, wherein the second DNA sequence is from a HIV-infected subject.

The invention additionally provides a composition comprising a vector that comprises, in operable combination, i) a DNA sequence encoding an HIV genome sequence containing a deletion of an HIV sequence, wherein the deleted HIV sequence is substituted by a heterologous sequence, and ii) a reporter gene. In one embodiment, the vector further comprises iii) a first restriction sequence and a second restriction sequence that flank the heterologous sequence. In yet another embodiment, the vector comprises pNL4-3-Δ(p24-VPR)-hRluc having SEQ ID NO:07. In a further embodiment, the deleted HIV sequence is substituted with a corresponding sequence from a HIV-infected subject.

The invention also provides a kit comprising (a) one or more compositions described herein, and (b) instructions for using the composition. In a particular embodiment, the kit contains a composition comprising a vector that a) lacks a DNA sequence encoding HIV 5′ long terminal repeat (LTR), and b) comprises an HIV genome sequence that contains, in place of a first DNA sequence encoding an HIV RNA sequence, i) a restriction sequence which can be specifically cleaved by a first restriction enzyme, and ii) a restriction sequence which can be specifically cleaved by a second restriction enzyme. In another embodiment, the kit contains a composition comprising a vector that comprises, in operable combination, i) a DNA sequence encoding an HIV genome sequence containing a deletion of an HIV sequence, wherein the deleted HIV sequence is substituted by a heterologous sequence, and ii) a reporter gene.

### BRIEF DESCRIPTION OF THE DRAWINGS

FIG. 1. Art methods to produce recombinant HIV. A. Schema of the most common methods to produce recombinant HIV. B. Complementation system to produce chimeric HIV using the yeast-base cloning method.

FIG. 2. Time of virus propagation in MT-4 cells to achieve enough yield (TCID₅₀≧10⁴ IU/ml) to run HIV-1 phenotypic assays.

FIG. 3. Comparing virus production using three different vectors and the complementation system.

FIG. 4. Construction of HIV-1 expressing renilla (hRluc) or firefly (fluc2) luciferase genes. (A) Replacing the EGFP gene in the p83-10-EGFP plasmid (5) with the luciferase genes. (B) Introduction of the luciferase genes into the pNL4-3-EGFP vector. (C) Schema of the resulting HIV-1 sequence with the hRluc or fluc2 genes between the Env and Nef open reading frames.

FIG. 5. (A) Replication kinetics of hRluc-expressing and fluc2-expressing viruses. (B) HIV-1-hRluc and HIV-1-fluc2 are able to infect a variety of cell lines.

FIG. 6. Drug susceptibility curves (A) and IC₅₀ determination comparison (B) using HIV-1 expressing either hRluc or fluc2 proteins.

FIG. 7. Predicted nucleotide sequence following the successful insertion of the SphI-SalI linker 5′-GCATGCGGCGCGCCGTCGAC-3′ (SEQ ID NO13) into the pNL4-3-hRluc vector.

FIG. 8. Sequenogram of the six clones tested. The fourth clone is the only one not correct.

FIG. 9. Schema of the “pNL4-3-Δ(SphI-SalI)-hRluc” vector, that is interchangeably named “pNL4-3-Δ(p24-VPR)-hRluc” since, in one embodiment, SphI cuts in p24 and SalI cuts in VPR. A schematic of pNL4-3-Δ(SphI-SalI)-hRluc is also shown in FIG. 10, and its DNA sequence SEQ ID NO:07 in FIG. 22.

FIG. 10. Schema of the production of p2-Int-recombinant viruses using the pNL4-3-Δ(SphI-SalI)-hRluc vector (also referred to herein as pNL4-3-Δ(p24-VPR)-hRluc). The DNA sequence of the p24-VPR fragment shown in FIG. 10 is listed as SEQ ID NO:05 (FIG. 20).

FIG. 11. Genotype (mutations) and phenotype (drug susceptibility) of the 08-188 p2-Int recombinant virus constructed using the single plasmid transfection approach based on the pNL4-3-Δ(SphI-SalI)-hRluc vector (also referred to herein as pNL4-3-Δ(p24-VPR)-hRluc).

FIG. 12. Comparing virus production using three different vectors and the complementation system (two vectors) versus a one vector transfection approach

FIG. 13. Turn-around-time of the HIV-1 drug susceptibility assay using the art's method (two vectors) and the invention's exemplary method (one vector).

FIG. 14. Schematic of HIV-1 genome.

FIG. 15. DNA sequence encoding the genome of exemplary HIV-1 strain HXB2 (SEQ ID NO:09).

FIG. 16. DNA sequence of the 5′ LTR (SEQ ID NO:01) deleted from the TRP vector.

FIG. 17. DNA sequence of an exemplary firefly (fluc2) luciferase gene (SEQ ID NO:02).

FIG. 18. DNA sequence of the p2-int fragment (SEQ ID NO:03) that was deleted from the TRP vector.

FIG. 19. DNA sequence of an exemplary Renilla (hRluc) luciferase gene (SEQ ID NO:04).

FIG. 20. DNA sequence of the p24-VPR fragment (SEQ ID NO:05) that was deleted in the pNL4-3Δ(p24-VPR)-hRluc vector. The DNA sequence of the pNL4-3-Δ(p24-VPR)-hRluc vector is shown in FIG. 22 (SEQ ID NO:07).

FIG. 21. DNA sequence of the pNL4-3 vector without reporter gene (SEQ ID NO:06).

FIG. 22. DNA sequence of the pNL4-3-Δ(p24-VPR)-hRluc vector (also referred to as pNL4-3-Δ(SphI-SalI)-hRluc) (SEQ ID NO:07).

FIG. 23. DNA sequence of “pRECnfl-TRP-Δ(p2-INT)/URA3-hRluc” (also referred to herein as “pRECnfl-TRP-Δp2-Int-hRluc”) (SEQ ID NO:08) that was used to introduce the patient-derived HIV fragment by yeast-based recombination. This vector contains the complete HIV-1 genome (NL4-3 strain) minus the 5′ LTR, minus the p2/p7/p1/p6 regions from the gag gene and the pol (protease, reverse transcriptase & integrase) gene, and minus a p2-Int 3,232 nt fragment. The p2-Int fragment corresponds to the p2/p7/p1/p6 from Gag+the pol (PR, RT, INT) gene.

### DEFINITIONS

To facilitate understanding of the invention, a number of terms are defined below.

The term “recombinant nucleotide sequence” refers to a nucleotide sequence (e.g., DNA, RNA) that is comprised of segments joined together by means of molecular biological techniques. A “recombinant amino acid sequence” refers to an amino acid sequence expressed by a recombinant nucleotide sequence.

A “chimeric” sequence (e.g., nucleotide sequence, polypeptide sequence) refers to a sequence that contains at least two sequences that are covalently linked together. The linked sequences may be derived from different sources (e.g., different organisms, different tissues, different cells, etc.) or may be different sequences from the same source.

“Correspond to,” “corresponding with” and grammatical equivalents when in reference to a first sequence (e.g., nucleotide sequence and/or amino acid sequence) that corresponds to a second sequence mean that the first and second sequences are homologous and/or have the same or similar biological function. For example, where a first DNA sequence is from a HIV-infected patient and spans the HIV integrase gene, then a second DNA sequence that “corresponds” to the first DNA sequence refers, in one embodiment, to a sequence that is homologous to the HIV-infected patient's integrase gene. In another embodiment, the second DNA sequence, which “corresponds” to the first DNA sequence, has the same or similar biological function as the HIV-infected patient's integrase gene.

The terms “flanking,” and “flank” when made in reference to a first and second nucleotide sequences in relation to a third nucleotide sequence mean that the first nucleotide sequence is linked to the 5′ end of the third sequence (in the presence or absence of intervening nucleotides), and the second nucleotide sequence is linked to the 3′ end of the third sequence (in the presence or absence of intervening nucleotides). For example, where first restriction sequence and a second restriction sequence flank a DNA sequence of interest, means that the first restriction sequence is linked to the 5′ end of the DNA sequence of interest (in the presence or absence of intervening nucleotides), and the second restriction sequence is linked to the 3′ end of the DNA of interest (in the presence or absence of intervening nucleotides).

The term “recombinant mutation” refers to a mutation that is introduced by means of molecular biological techniques. This is in contrast to mutations that occur in nature.

The terms “endogenous” and “wild type” when in reference to a sequence refer to a sequence that is naturally found, e.g., in a cell or virus. An endogenous sequence in a virus includes a sequence that is found in the virus in the absence of selection by man-made agents (e.g., antiviral therapeutics or vaccines). The term “heterologous” refers to a sequence that is not endogenous to the cell or virus, but rather contains one or more mutation relative to the naturally occurring sequence. A heterologous sequence is exemplified by a linker sequence and lethal gene sequence, as described below.

The term “recombinant virus” refers to a virus that contains a recombinant DNA molecule, recombinant protein and/or recombinant mutation, as well as progeny of that virus.

The terms “mutation” and “modification” refer to a deletion, insertion, or substitution. A “deletion” is defined as a change in a nucleic acid sequence or amino acid sequence in which one or more nucleotides or amino acids, respectively, is absent. An “insertion” or “addition” is that change in a nucleic acid sequence or amino acid sequence that has resulted in the addition of one or more nucleotides or amino acids, respectively. An insertion also refers to the addition of any synthetic chemical group, such as those for increasing solubility, dimerization, binding to receptors, binding to substrates, resistance to proteolysis, and/or biological activity of the amino acid sequence. A “substitution” in a nucleic acid sequence or an amino acid sequence results from the replacement of one or more nucleotides or amino acids, respectively, by a molecule that is a different molecule from the replaced one or more nucleotides or amino acids. For example, a nucleic acid may be replaced by a different nucleic acid as exemplified by replacement of a thymine by a cytosine, adenine, guanine, or uridine. Alternatively, a nucleic acid may be replaced by a modified nucleic acid as exemplified by replacement of a thymine by thymine glycol. Substitution of an amino acid may be conservative or non-conservative. A “conservative substitution” of an amino acid refers to the replacement of that amino acid with another amino acid that has a similar hydrophobicity, polarity, and/or structure. For example, the following aliphatic amino acids with neutral side chains may be conservatively substituted one for the other: glycine, alanine, valine, leucine, isoleucine, serine, and threonine. Aromatic amino acids with neutral side chains that may be conservatively substituted one for the other include phenylalanine, tyrosine, and tryptophan. Cysteine and methionine are sulphur-containing amino acids, which may be conservatively substituted one for the other. Also, asparagine may be conservatively substituted for glutamine, and vice versa, since both amino acids are amides of dicarboxylic amino acids. In addition, aspartic acid (aspartate) may be conservatively substituted for glutamic acid (glutamate) as both are acidic, charged (hydrophilic) amino acids. Also, lysine, arginine, and histidine may be conservatively substituted one for the other since each is a basic, charged (hydrophilic) amino acid. “Non-conservative substitution” is a substitution other than a conservative substitution. Guidance in determining which and how many amino acid residues may be substituted, inserted or deleted without abolishing biological and/or immunological activity may be found using computer programs well known in the art, for example, DNAStar™ software.

The invention contemplates homologs of each and every one of the sequences and portions described herein. “Homolog” and “variant” of a sequence of interest interchangeably refer to a sequence that differs by at least one insertion, deletion, and/or substitution from the sequence of interest. In one embodiment, a homolog of a sequence of interest has from 95% to 100% identity (including from 96% to 100%, from 97% to 100%, from 98% to 100%, from 99% to 100%) to the sequence of interest. In another embodiment, where the sequence of interest is a DNA sequence, a homolog of the DNA sequence includes sequences that hybridize under high stringent conditions to the DNA sequence. “High stringency conditions” when used in reference to nucleic acid hybridization comprise conditions equivalent to binding or hybridization at 42° C. in a solution of 5× SSPE (43.8 g/l NaCl, 6.9 g/l NaH₂PO₄—H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 0.1× SSPE, 1.0% SDS at 42° C. when a probe of about 500 nucleotides in length is employed. In another embodiment, high stringency conditions comprise conditions equivalent to binding or hybridization at 68° C. in a solution containing 5× SSPE, 1% SDS, 5× Denhardt's reagent and 100 μg/ml denatured salmon sperm DNA followed by washing in a solution containing 0.1× SSPE, and 0.1% SDS at 68° C. when a probe of about 100 to about 1000 nucleotides in length is employed.

“Portion” when made in reference to a sequence refers to a fragment of that sequence. The fragment may range in size from 2 contiguous residues to the entire sequence minus one residue. Thus, a nucleic acid sequence comprising “at least a portion of” a first nucleotide sequence comprises from two (2) nucleotide residue of the first nucleotide sequence to the entire first nucleotide sequence. Also, an amino acid sequence comprising “at least a portion of”a first amino acid sequence comprises from two (2) amino acid residues of the first amino acid sequence to the entire first amino acid sequence.

“Operable combination” and “operably linked” when in reference to the relationship between nucleic acid sequences and/or amino acid sequences refer to linking the sequences such that they perform their intended function. For example, operably linking a promoter sequence to a nucleotide sequence of interest refers to linking the promoter sequence and the nucleotide sequence of interest in a manner such that the promoter sequence is capable of directing the transcription of the nucleotide sequence of interest and/or the synthesis of a polypeptide encoded by the nucleotide sequence of interest.

“Amplification” of a target nucleotide sequence refers to the production of multiple copies of the target sequence. Nucleic acid sequences may be amplified by techniques such as polymerase chain reaction (PCR), nucleic acid sequence based amplification (NASBA), self-sustained sequence replication (3SR), transcription-based amplification (TAS), ligation chain reaction (LCR). In one preferred embodiment, amplification uses a “polymerase chain reaction” (“PCR”), which refers to the method of K. B. Mullis that is disclosed in U.S. Pat. Nos. 4,683,195, 4,683,202 and 4,965,188, and that describes a method for increasing the concentration of a segment of a target sequence in a mixture of DNA sequences without cloning or purification.

“Amplicon” refers to a nucleic acid sequence that has been amplified.

“Genotype” is the genetic composition of a cell, an organism, or an individual (i.e. the specific allele makeup of the individual), usually with reference to a specific character under consideration. Inherited genotype, transmitted epigenetic factors, and non-hereditary environmental variation contribute to the “phenotype”, i.e., any observable characteristic or trait, such as its morphology, development, biochemical properties, physiological properties, and/or behavior. Genotype differs subtly from genomic sequence. A sequence is an absolute measure of base composition of an individual, or a representative of a species or group. In contrast, a genotype typically implies a measurement of how an individual differs from, or is specialized within, a group of individuals or a species. So typically, one refers to a cell's genotype with regard to a particular gene of interest. In polyploid individuals, genotype refers to the combination of alleles. Methods for determining genotype are known in the art, including PCR, DNA sequencing, Allele Specific Oligonucleotide (ASO) probes, and hybridization to DNA microarrays or beads.

“Subject” and “animal” interchangeably refer to any multicellular animal, preferably a mammal, e.g., humans, non-human primates, murines, ovines, bovines, ruminants, lagomorphs, porcines, caprines, equines, canines, felines, ayes, etc.). Thus, mammalian subjects include mouse, rat, guinea pig, hamster, ferret and chinchilla.

“Propagation” of a virus refers to the release of virus particles from a cell (such as a producer cell) into culture medium. A “producer cell” is a cell that is susceptible to a virus, and is capable of releasing replication-competent and/or replication-incompetent viral particles into culture medium.

The term “susceptible” as used herein in reference to a cell that is susceptible to a virus describes the ability of a permissive or non-permissive host cell to be infected by the virus. Susceptibility of a cell may be determined by detection in the cell of viral proteins and/or viral nucleic acids (including both RNA and DNA), by release of progeny virus into the culture medium, and/or by observation of a cytopathic effect. HIV-susceptible cells include cells (e.g., primary cell, cell line, etc.) that express the receptor CD4 and/or CXCR4 and/or CCR5, and are exemplified by the cells MT-4, MT-2, PM1, HUT78, 174xCEM, CEM.CCR5.CXCR4, U87.CD4.CXCR4, U87.CD4.CCR5, GHOSTX4/R5, and TZM-bl, T cells, etc.

“CXCR4” (also referred to as “fusin”) and “CCR5” are both chemokine receptor proteins normally embedded in the membrane of a cell. HIV-1 is able to use either CXCR4 or CCR5 as a co-receptor CD4 being the main receptor) to facilitate binding and entry into T cells. HIV strains that use CXCR4 are called “X4”, while HIV strains that use CCR5 are called “R5.” “Infection” refers to adsorption of the virus to the cell and penetration into the cell. A cell may be susceptible without being permissive in that a virus can penetrate it in the absence of viral replication and/or release of virions from the cell. A permissive cell line however must be susceptible. Susceptibility of a cell to a virus may be determined by methods known in the art such as detecting the presence of viral proteins using electrophoretic analysis (i.e., SDS-PAGE) of protein extracts prepared from the infected cell cultures. Susceptibility to a retrovirus may also be determined by detecting the presence of retroviral RNA.

The terms “permissive” and “permissiveness” as used herein describe the sequence of interactive events between a virus and its putative host cell. The process begins with viral adsorption to the host cell surface and ends with release of infectious virions. A cell is “permissive” (i.e., shows “permissiveness”) if it is capable of supporting viral replication as determined by, for example, production of viral nucleic acid sequences and/or of viral peptide sequences, regardless of whether the viral nucleic acid sequences and viral peptide sequences are assembled into a virion. While not required, in one embodiment, a cell is permissive if it generates virions and/or releases the virions contained therein. Many methods are available for the determination of the permissiveness of a given cell line. For example, the replication of a particular virus in a host cell line may be measured by the production of various viral markers including viral proteins, viral nucleic acid (including both RNA and DNA) and the progeny virus. The presence of viral proteins may be determined using electrophoretic analysis (i.e., SDS-PAGE) of protein extracts prepared from the infected cell cultures. Viral nucleic acid sequences may be quantitated using nucleic acid hybridization assays. Production of progeny virus may also be determined by observation of a cytopathic effect. However, in some embodiments, this method may be less preferred than detection of viral nucleic acid sequences, since a cytopathic effect may not be observed even when viral replication is detectable by the presence of viral nucleic acid sequences. The invention is not limited to the specific quantity of replication of virus.

The terms “not permissive” and “non-infections” encompasses, for example, a cell that is not capable of supporting viral replication as determined by, for example, production of viral nucleic acid sequences and/or of viral peptide sequences, and/or assembly of viral nucleic acid sequences and viral peptide sequences into a virion.

The term “viral proliferation” as used herein describes the spread or passage of infectious virus from a permissive cell to additional cells of either a permissive or susceptible character.

The terms “cytopathic effect” and “CPE” as used herein describe changes in cellular structure (i.e., a pathologic effect). Common cytopathic effects include cell destruction, syncytia (i.e., fused giant cells) formation, cell rounding, vacuole formation, and formation of inclusion bodies.

The terms “reduce,” “inhibit,” “diminish,” “suppress,” “decrease,” and grammatical equivalents (including “lower,” “smaller,” etc.) when in reference to the level of any molecule (e.g., amino acid sequence, and nucleic acid sequence such as those encoding any of the polypeptides described herein), cell, viral particle, and/or phenomenon (e.g., viral infection, viral replication, viral propagation, disease symptom, binding to a molecule, affinity of binding, expression of a nucleic acid sequence, transcription of a nucleic acid sequence, enzyme activity, etc.) in a first sample (or in a first subject) relative to a second sample (or relative to a second subject), mean that the quantity of molecule, cell and/or phenomenon in the first sample (or in the first subject) is lower than in the second sample (or in the second subject) by any amount that is statistically significant using any art-accepted statistical method of analysis. In one embodiment, the quantity of molecule, cell and/or phenomenon in the first sample (or in the first subject) is at least 10% lower than, at least 25% lower than, at least 50% lower than, at least 75% lower than, and/or at least 90% lower than the quantity of the same molecule, cell and/or phenomenon in the second sample (or in the second subject). In another embodiment, the quantity of molecule, cell, and/or phenomenon in the first sample (or in the first subject) is lower by any numerical percentage from 5% to 100%, such as, but not limited to, from 10% to 100%, from 20% to 100%, from 30% to 100%, from 40% to 100%, from 50% to 100%, from 60% to 100%, from 70% to 100%, from 80% to 100%, and from 90% to 100% lower than the quantity of the same molecule, cell and/or phenomenon in the second sample (or in the second subject). In one embodiment, the first subject is exemplified by, but not limited to, a subject to whom the invention's compositions have been administered. In a further embodiment, the second subject is exemplified by, but not limited to, a subject to whom the invention's compositions have not been administered. In an alternative embodiment, the second subject is exemplified by, but not limited to, a subject to whom the invention's compositions have been administered at a different dosage and/or for a different duration and/or via a different route of administration compared to the first subject. In one embodiment, the first and second subjects may be the same individual, such as where the effect of different regimens (e.g., of dosages, duration, route of administration, etc.) of the invention's compositions is sought to be determined in one individual. In another embodiment, the first and second subjects may be different individuals, such as when comparing the effect of the invention's compositions on-one individual participating in a clinical trial and another individual in a hospital.

The terms “increase,” “elevate,” “raise,” and grammatical equivalents (including “higher,” “greater,” etc.) when in reference to the level of any molecule (e.g., amino acid sequence, and nucleic acid sequence such as those encoding any of the polypeptides described herein), cell, viral particle, and/or phenomenon (e.g., viral infection, viral replication, viral propagation, disease symptom, binding to a molecule, affinity of binding, expression of a nucleic acid sequence, transcription of a nucleic acid sequence, enzyme activity, etc.) in a first sample (or in a first subject) relative to a second sample (or relative to a second subject), mean that the quantity of the molecule, cell and/or phenomenon in the first sample (or in the first subject) is higher than in the second sample (or in the second subject) by any amount that is statistically significant using any art-accepted statistical method of analysis. In one embodiment, the quantity of the molecule, cell and/or phenomenon in the first sample (or in the first subject) is at least 10% greater than, at least 25% greater than, at least 50% greater than, at least 75% greater than, and/or at least 90% greater than the quantity of the same molecule, cell and/or phenomenon in the second sample (or in the second subject). This includes, without limitation, a quantity of molecule, cell, and/or phenomenon in the first sample (or in the first subject) that is at least 10% greater than, at least 15% greater than, at least 20% greater than, at least 25% greater than, at least 30% greater than, at least 35% greater than, at least 40% greater than, at least 45% greater than, at least 50% greater than, at least 55% greater than, at least 60% greater than, at least 65% greater than, at least 70% greater than, at least 75% greater than, at least 80% greater than, at least 85% greater than, at least 90% greater than, and/or at least 95% greater than the quantity of the same molecule, cell and/or phenomenon in the second sample (or in the second subject).

“Alter” and “change”mean increase or decrease.

“Substantially the same” and “substantially similar” mean without an increase and without a decrease.

Reference herein to any numerical range expressly includes each numerical value (including fractional numbers and whole numbers) encompassed by that range. To illustrate, and without limitation, reference herein to a range of “at least 50” includes whole numbers of 50, 51, 52, 53, 54, 55, 56, 57, 58, 59, 60, etc., and fractional numbers 50.1, 50.2 50.3, 50.4, 50.5, 50.6, 50.7, 50.8, 50.9, etc. In a further illustration, reference herein to a range of “less than 50” includes whole numbers 49, 48, 47, 46, 45, 44, 43, 42, 41, 40, etc., and fractional numbers 49.9, 49.8, 49.7, 49.6, 49.5, 49.4, 49.3, 49.2, 49.1, 49.0, etc. In yet another illustration, reference herein to a range of from “5 to 10” includes each whole number of 5, 6, 7, 8, 9, and 10, and each fractional number such as 5.1, 5.2, 5.3, 5.4, 5.5, 5.6, 5.7, 5.8, 5.9, etc.

### BRIEF DESCRIPTION OF THE INVENTION

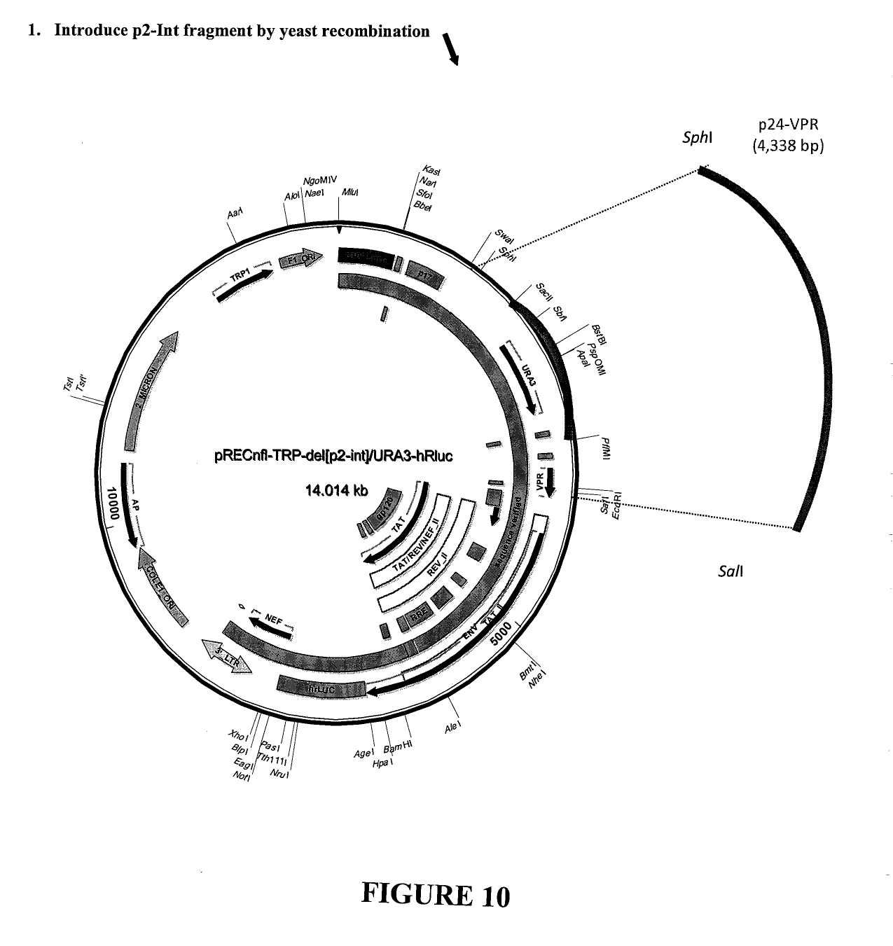

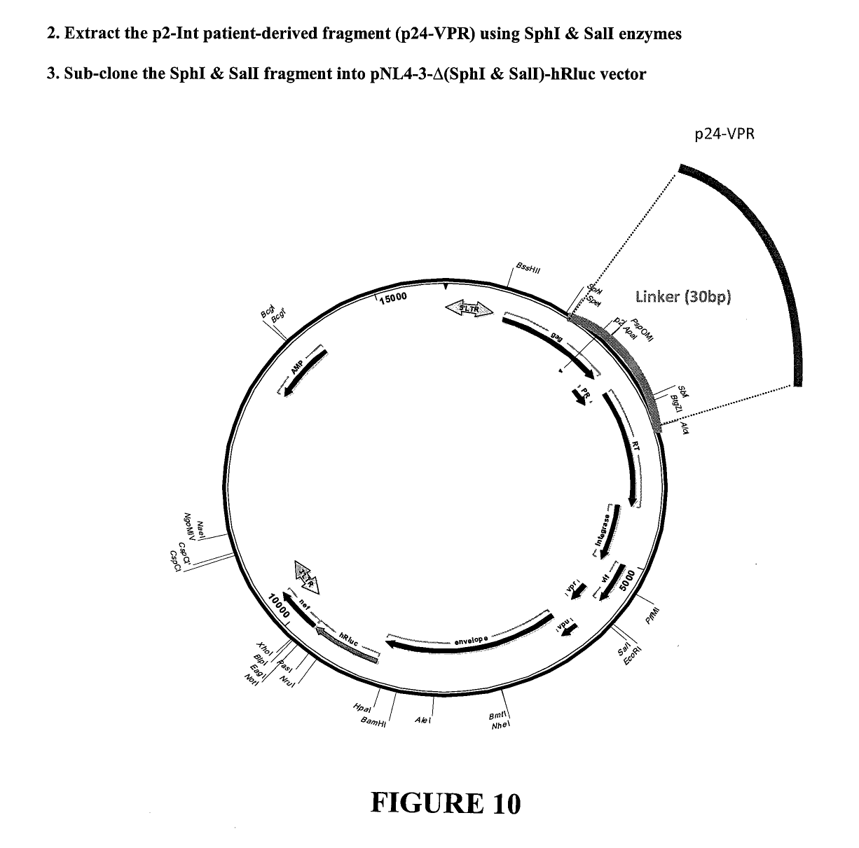

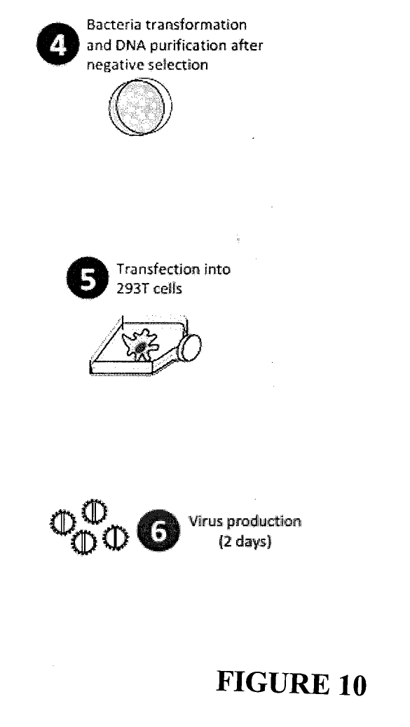

The invention provides a more efficient system than the prior art's systems to construct recombinant HIV expressing reporter genes, as summarized in the Exemplary FIG. 10. In one embodiment, the invention's methods introduce a patient-derived HIV genomic fragment into a vector (lacking the 5′LTR and the complementary HIV sequence) by homologous recombination in yeast cells. By doing this, the invention takes advantage of the unique feature of homologous recombination in yeast, which allows the cloning of one, two, or more overlapping DNA fragments into a single vector. A fragment spanning the patient-derived HIV genomic sequence is then transferred into a second vector (devoid of the complementary HIV sequence but containing a reporter gene without affecting the expression of any viral gene) by restriction enzymes and ligation. This vector carries a polylinker instead of the HIV sequence complementary to the patient-derived HIV sequence to be cloned, and/or a positive selection (lethal) gene to guarantee the growth only of clones carrying the patient-derived HIV sequence. This resulting single vector is transfected into HEK 293T-cells to produce high titers of fully infectious recombinant virus in two days. HIV replication can then be evaluated by multiple methods (e.g., reverse transcriptase or p24 EIA assays), including the expression of the intrinsic reporter gene.

The invention provides the development of a novel phenotypic assay to quantify antiretroviral resistance and construction of chimeric viruses tagged with reporter genes. In one embodiment, the inventors introduced the renilla luciferase (hRluc) gene between the Env and Nef open reading frames (5,6). In addition, the inventors modified the pRECnfl-LEU-HIV-1Δgene/URA3 by deleting non-essential components and created the pRECnfl-AK-HIV-1Δgene/URA3. The invention's vector expressing the renilla luciferase gene was then named pRECnfl-AK-HIV-1Δgene/URA3-hRluc.

### DETAILED DESCRIPTION OF THE INVENTION

The invention provides methods for producing replication competent chimeric human immunodeficiency viruses (HIV) that contain a heterologous reporter gene, and methods for generating these viruses. The invention's recombinant viruses are useful in the determination of, for example, antiretroviral drug susceptibility, HIV drug resistance, HIV phenotyping, HIV genotyping, HIV fitness, HIV tropism or coreceptor usage, HIV serum neutralization, and for HIV vaccine development, HIV vector development, and HIV virus production.

Thus, in one embodiment, the invention provides a method to produce fully infectious HIV recombinant viruses expressing reporter genes without deleting or altering the expression of any viral gene. The method allows the rapid and efficient cloning of an amplicon into an HIV genome vector devoid of at least a portion of the sequence for the 5′ long terminal repeat region through recombination/gap repair in organisms such as yeast. A sequence containing the amplicon is then cloned into an HIV genome vector through restriction enzyme digestion and ligation in organisms such as bacteria. The invention's single vector can be passed to a mammalian cell line which has been specifically engineered to produce replication competent HIV-1 particles.

The invention's novel methods for constructing HIV recombinant viruses expressing a reporter gene are more efficient than the prior art methods for determining HIV phenotype with respect to drug resistance, because it allows, in some embodiment, targeting of multiple HIV genes (such as gag, protease, reverse transcriptase, and integrase) and produces multi-gene screening in a single assay. Thus, the invention's novel assays are useful as a companion diagnostic modality that provides the most personalized and efficacious anti-HIV treatment regimen to-date.

The recombinant viruses produced by the invention's methods are useful in multiple applications such as (i) HIV vector development, (ii) HIV production, (iii) antiretroviral drug susceptibility, (iv) HIV drug resistance, (v) HIV phenotyping, (vi) HIV genotyping, (vii) HIV fitness determination, (viii) HIV coreceptor tropism, (ix) HIV serum neutralization, (x) HIV vaccine development, and (xi) other applications that utilize HIV. Thus, in one embodiment, high-throughput assays may be used to amplify a virus population from a patient, and use it to quantify the virus' resistance to available drugs. This may be accomplished by analyzing the replicative fitness of recombinant HIV-1 viruses, which express one or more chimeric reporter gene and which are derived from a subject, in the presence and absence of a drug (e.g., anti-retroviral drug), and correlating the results to in vivo treatment. In another embodiment, the recombinant viruses produced by the invention's methods may be used to analyze the effect of one or more mutations in one or more HIV-genes on HIV-1 transmission, replication, and/or pathogenesis.

The invention is further described under (A) the art's methods for constructing recombinant HIV, and (B) the invention's methods for constructing recombinant HIV.

### A. The Art's Methods for Constructing Recombinant HIV

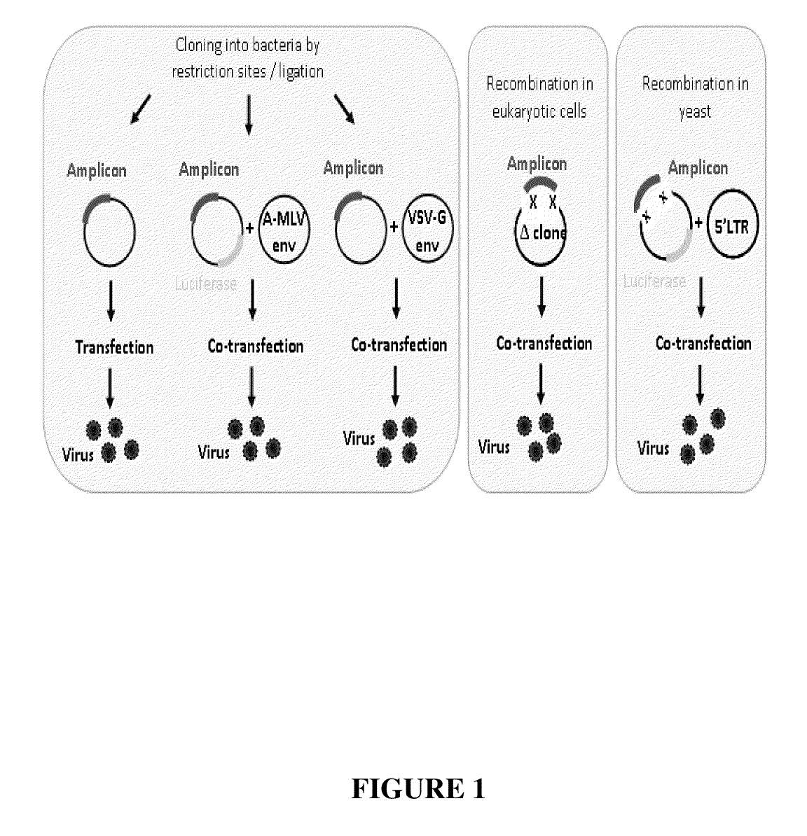

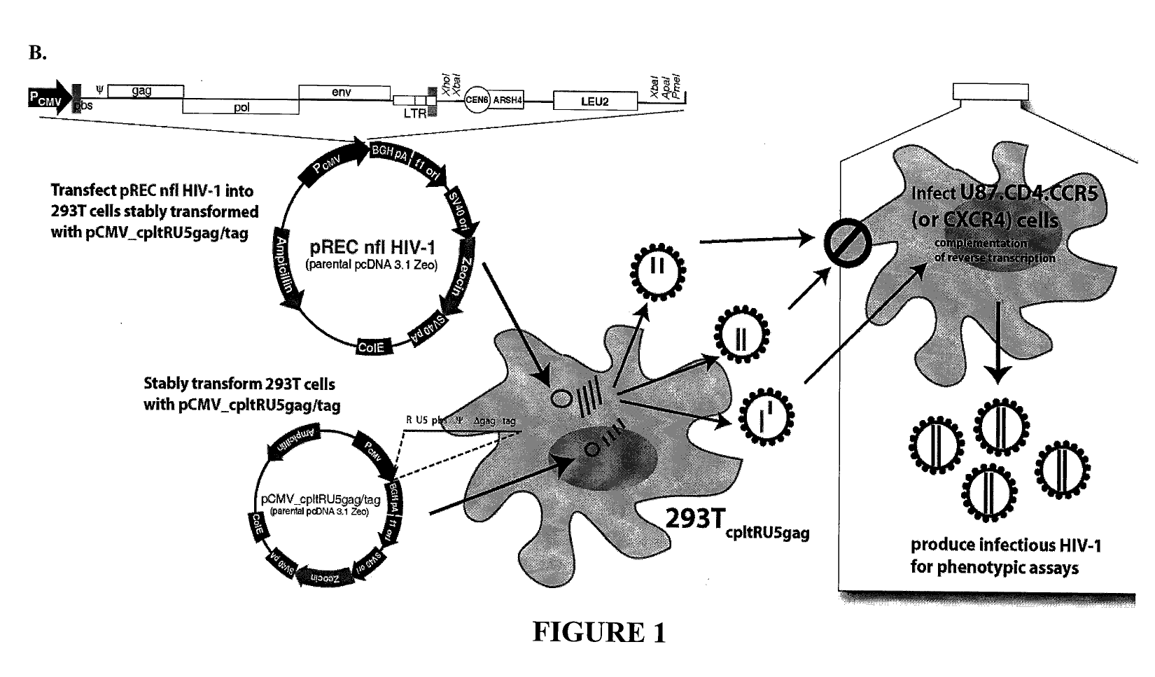

During the more than 25 years following the discovery of the HIV as the agent causing AIDS, multiple approaches have been evaluated to study this virus in vitro. Most of them involve the construction of recombinant viruses carrying fragment(s) of the HIV genome obtained from clinical samples. These methodologies can be summarized in three basic systems (FIG. 1A): Cloning into bacteria using restriction enzymes and ligation, homologous recombination in mammalian cells, and homologous recombination in yeast cells. Each of the prior art's method has disadvantages.

The yeast-based recombination method to clone and propagate HIV-1 strains has been described (Dudley et al. (2009); U.S. Patent Pub. No.: US 2009/0130654 A1). Briefly, the method involves extraction of HIV-1 RNA from plasma samples (or any other source of HIV-1), and a HIV-1 fragment is RT-PCR amplified. This PCR product is co-transformed into yeast together with the pRECnfl-LEU-HIV-1Δgene/URA3 vector. Recombinant plasmids are selected on C-leu-/FOA plates or media. The recombined plasmid (pREC_nfl HIV-1geneₚₐₜᵢₑₙₜ) is extracted from yeast and transformed into bacteria to increase the DNA yield. Plasmid DNA extracted from bacteria is used to co-transfect 293T cells together with pCMV_cpltRU5gag plasmid (carrying the 5′LTR of HIV-1). Virus produced from HEK 293T cells is propagated by infecting HIV-susceptible cells such as U87.CD4.CCR5, U87.CD4.CXCR4, or MT-4 cells, followed by determination of virus titer (TCID₅₀). A schema summarizing this process is depicted in FIG. 1B. As described by Dudley et al (2), this system was originally designed to construct recombinant viruses without the expression of any reporter gene.

However, yeast recombination as used in the art's above method creates a substantial drawback. As described above, the producer cells (HEK 293T) need to be co-transfected with two plasmids, i.e., one containing the Gag to 3′LTR sequence of the HIV-1 genome and a second one that provides the 5′LTR to complete reverse transcription and produce infectious virions. This complementation event has proven to be extremely variable, especially with viruses harboring multiple drug resistance mutations (impaired fitness) and expressing reporter genes such as human renilla luciferase (hRluc). Therefore, recombinant viruses need to be propagated in another cell line (e.g., MT-4 cells) for a period of time ranging from 5 to 28 days. In some cases, even after a month, no virus replication is detected.

### B. The Invention's Methods for Constructing Recombinant HIV

The invention's methods are described under 1. Human immunodeficiency virus (HIV), 2. Preliminary data, 3. Exemplary methods for producing reporter-tagged HIV particles, 4. Reporter genes, 5. Vectors, 6. Restriction sequences, 7. Phenotyping and genotyping, and 8. Kits.

1. Human Immunodeficiency Virus (HIV)

The invention's methods are useful for producing recombinant HIV particles. “Human immunodeficiency virus” and “HIV” refer to a retrovirus that can lead to acquired immunodeficiency syndrome (AIDS), a condition in humans in which the immune system begins to fail, leading to life-threatening opportunistic infections. HIV includes HIV-1 and HIV-2, both of which infect humans. HIV-1 is the virus that was initially discovered and termed LAV. It is more virulent, relatively easily transmitted, and is the cause of the majority of HIV infections globally. HIV-2 is less transmittable than HIV-1 and is largely confined to West Africa. “HIV” includes primary virus that is isolated from infected subjects, and cultured virus that is passaged in vivo and/or in vitro.

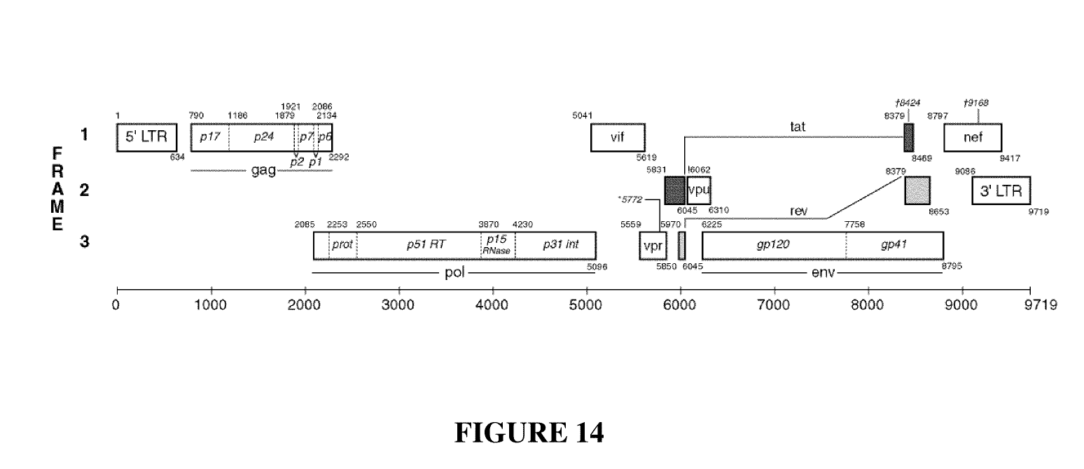

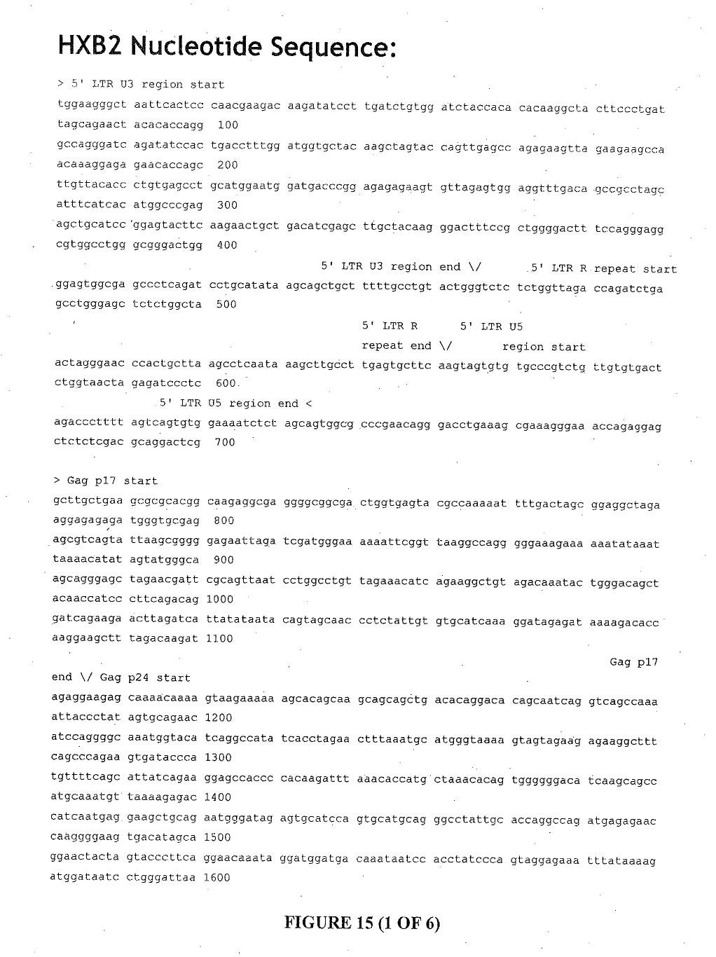

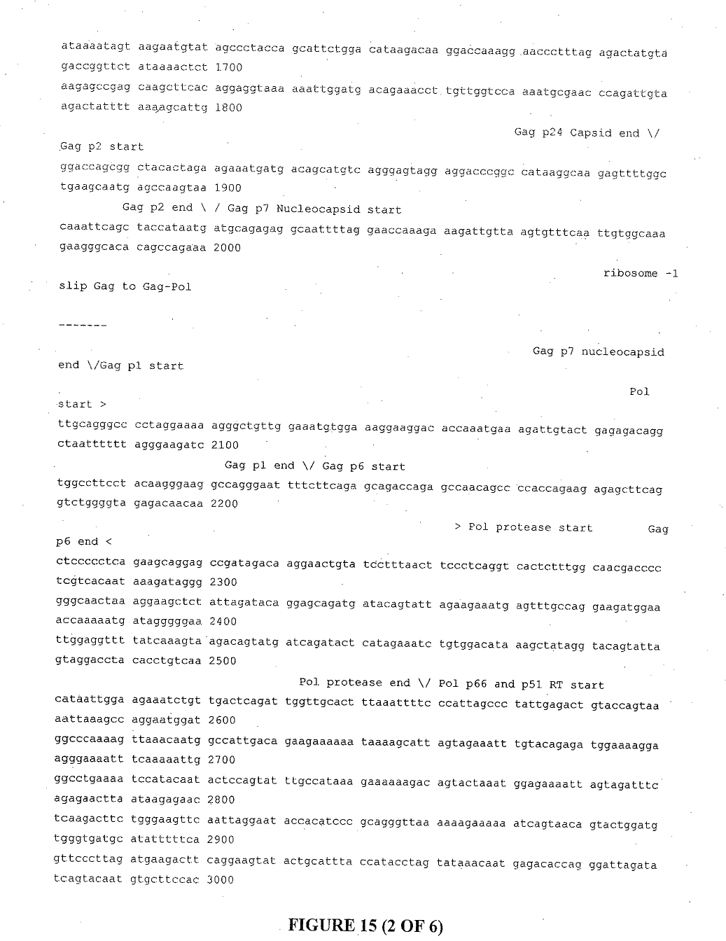

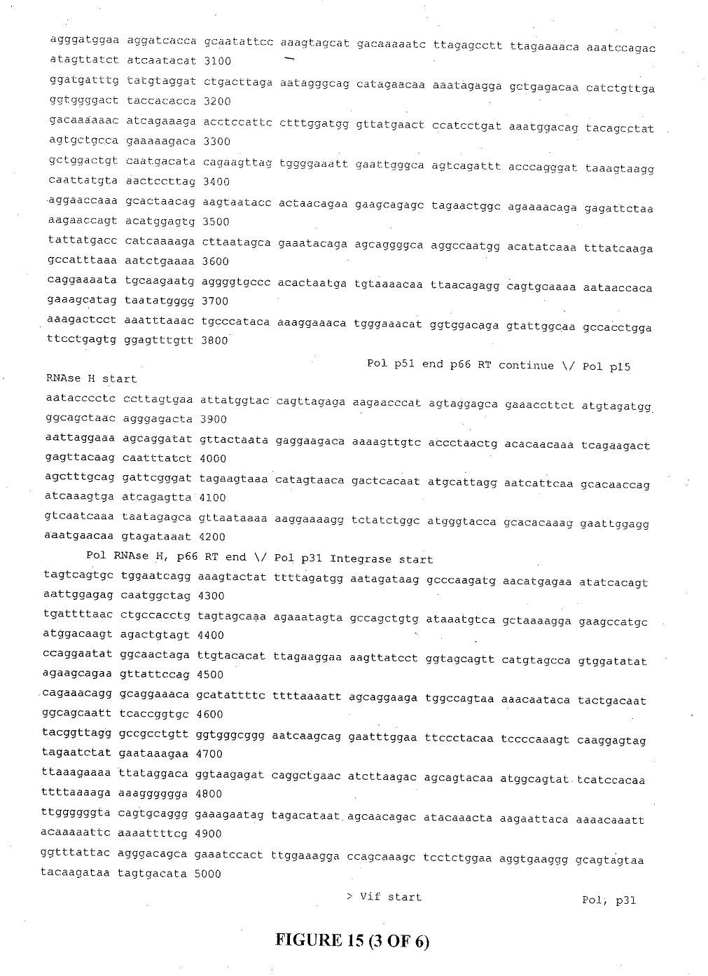

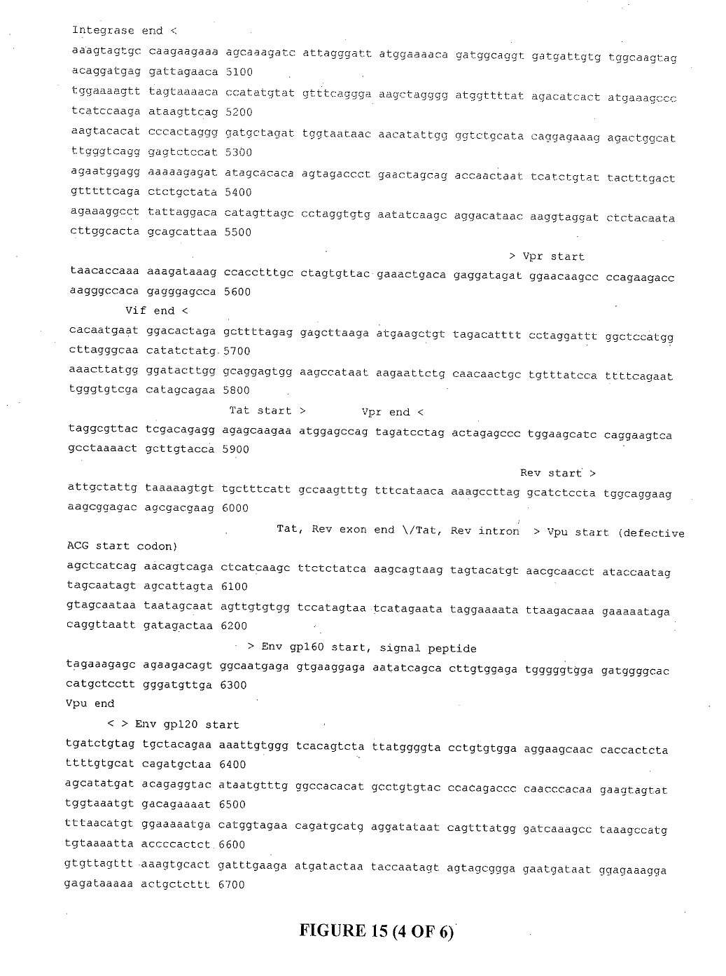

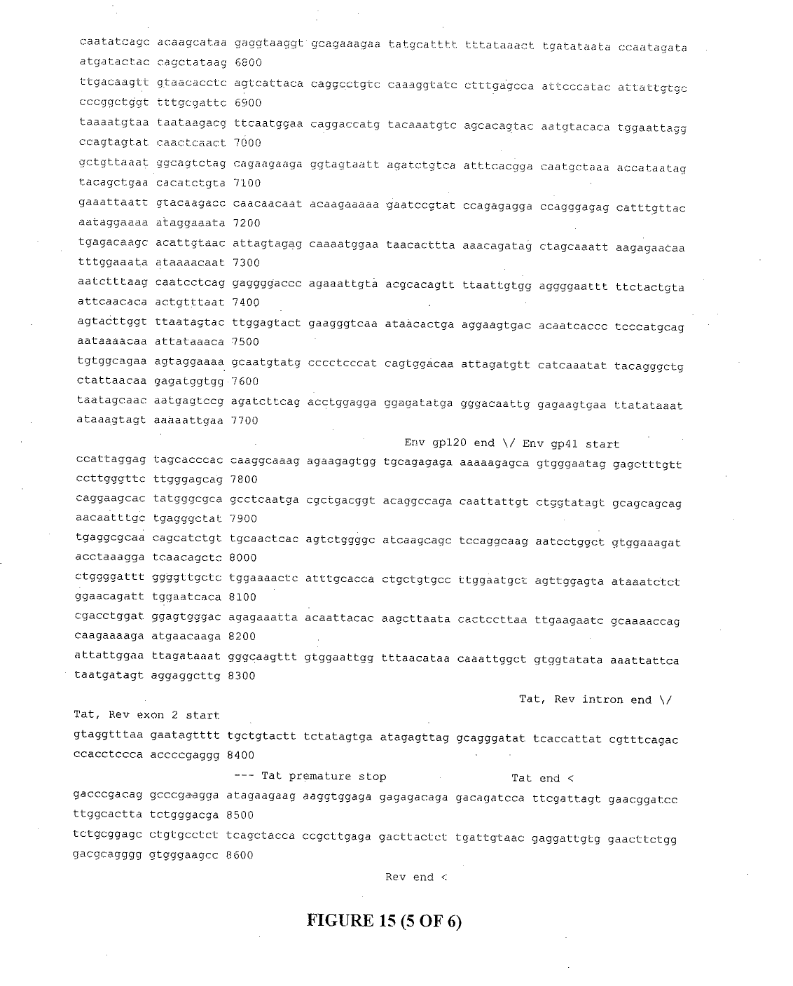

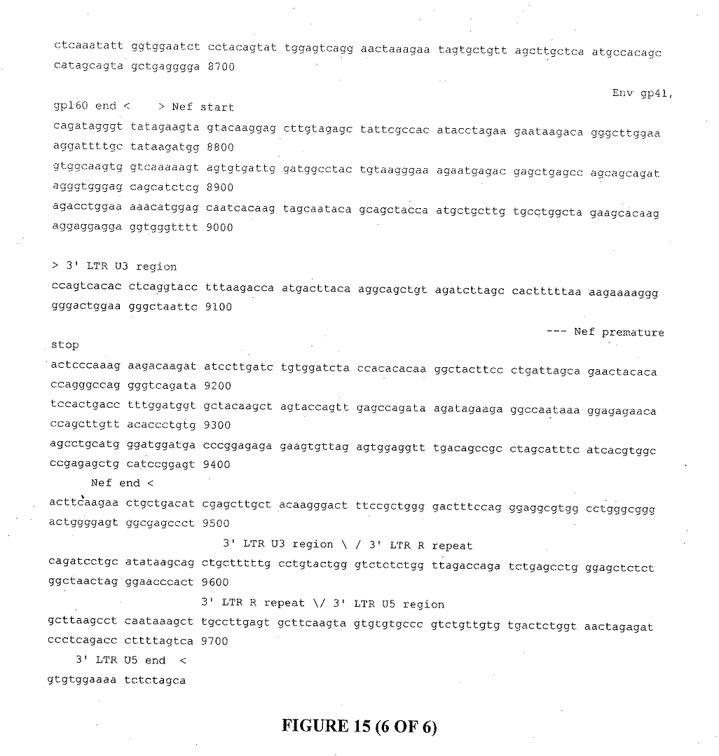

“HIV-1” is exemplified by a virus having a genome structure (FIG. 14) and/or having a nucleotide sequence that has from 80% to 100% identity (including any numerical value from 80% to 100%, such as 81%, 82%, 83%, 84%, 85%, 86%, 87%, 88%, 89%, 90%, 91%, 92%, 93%, 94%, 95%, 96%, 97%, 98%, and 99%) to strain HXB2D (GenBank accession number K03455) (SEQ ID NO:09 of FIG. 15).

“HIV-2” is exemplified by a virus having a genome structure and/or having a nucleotide sequence that has from 80% to 100% identity (including any numerical value from 80% to 100%, such as 81%, 82%, 83%, 84%, 85%, 86%, 87%, 88%, 89%, 90%, 91%, 92%, 93%, 94%, 95%, 96%, 97%, 98%, and 99%) to strain Mac239 (GenBank accession number M33262.1)

One skilled in the art understands that HIV may contain one or more mutations compared to a reference sequence, such as that of HXB2D (SEQ ID NO:09 of FIG. 15). “HIV” may be R5-tropic, X4-tropic, or R5X4-tropic. A “R5-tropic strain” refers to a virus strain that uses CCR5 co-receptor in the fusion process, exemplified by, but not limited to ADA, Ba-L, UCS, SF162, NLBa1, JRCSF, YU2.c, 92US715, and CC1/85. A “X4-tropic strain” refers to a virus strain that uses CXCR4 co-receptor in the fusion process, such as NL4-3, HXB2, and HXB3. A “R5X4-tropic strain” refers to a virus strain that uses both CCR5 and CXCR4 co-receptors in the fusion process, such as 89.6 strain. In general, R5-tropic strains are nearly exclusively present during acute infection with HIV and the asymptomatic phase, while X4-tropic viruses are involved in later stages of HIV infection.

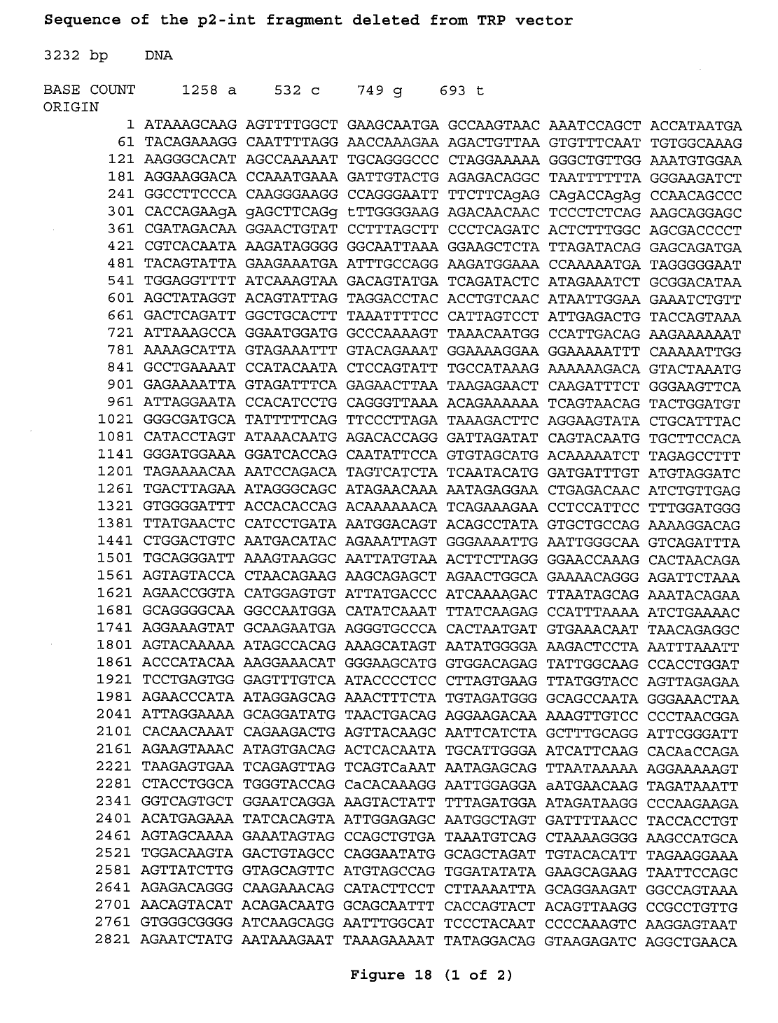

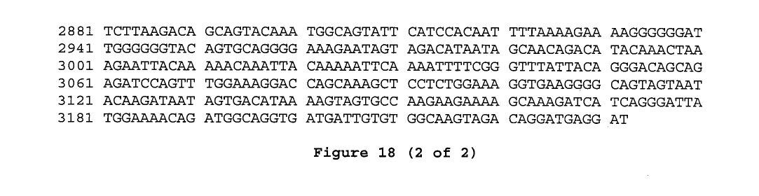

“HIV RNA sequence” refers to at least a portion of HIV RNA genome. “HIV genome” and “HIV RNA genome” are used interchangeably to include HIV genes and HIV genomic structural elements. Thus, an HIV RNA sequence includes coding sequences and portions thereof, non-coding sequences and portions thereof, full genes and portions thereof, structural elements and portions thereof, etc. An exemplary HIV genome is illustrated by the schematic (FIG. 14) and DNA sequence (SEQ ID NO:09 of FIG. 15) encoding it for strain HXB2D. In one embodiment, an HIV RNA sequence from a subject infected with HIV is used in the invention's methods, as exemplified by the sequence encoded by the DNA SEQ ID NO:03 of FIG. 18.

“HIV gene” refers to one or more of gag, pol, env, tat, rev, vif, vpr, vpu, nef, and vpx genes.

The “gag” gene encodes the capsid proteins Gag (group specific antigens). The precursor is the p55 myristylated protein, which is processed to p17 (MAtrix), p24 (CApsid), p7 (NucleoCapsid), and p6 proteins, by the viral protease. Gag associates with the plasma membrane, where virus assembly takes place. The 55-kDa Gag precursor is called “assemblin” to indicate its role in viral assembly.

The “pol” gene encodes the viral enzymes protease, reverse transcriptase, and integrase. These enzymes are produced as a Gag-Pol precursor polyprotein, which is processed by the viral protease; the Gag-Pol precursor is produced by ribosome frame shifting near the 3′ end of gag.

The “env” gene encodes Env, viral glycoproteins produced as a precursor (gp 160), which is processed to give a non-covalent complex of the external glycoprotein gp120 and the transmembrane glycoprotein gp41. The “tat” gene encodes Tat, trans-activator of HIV gene expression, is one of two essential viral regulatory factors (Tat and Rev) for HIV gene expression. Two forms are known, Tat-1 exon (minor form) of 72 amino acids and Tat-2 exon (major form) of 86 amino acids.

The “rev” gene encodes Rev, the second necessary regulatory factor for HIV expression. Rev is a 19-1(D phosphoprotein, localized primarily in the nucleolus/nucleus, and acts by binding to RRE and promoting the nuclear export, stabilization, and utilization of the viral mRNAs containing RRE.

The “vif” gene encodes Vif, viral infectivity factor, a basic protein typically 23 kD, that promotes the infectivity but not the production of viral particles. In the absence of Vif, the produced viral particles are defective, while the cell-to-cell transmission of virus is not affected significantly. Found in almost all lentiviruses, Vif is a cytoplasmic protein, existing in both a soluble cytosolic form and a membrane-associated form. The latter form of Vif is a peripheral membrane protein that is tightly associated with the cytoplasmic side of cellular membranes.

The “vpr” gene encodes Vpr, viral protein R, that is a 96-amino acid (14-kD) protein, which is incorporated into the virion. It interacts with the p6 Gag part of the Pr55 Gag precursor. Vpr detected in the cell is localized to the nucleus. Proposed functions for Vpr include targeting the nuclear import of pre-integration complexes, cell growth arrest, trans-activation of cellular genes, and induction of cellular differentiation.

The “vpu” gene encodes Vpu, viral protein U, that is unique to HIV-1, SIVcpz (the closest SIV relative of HIV-1), SIV-GSN, SIV-MUS, SIV-MON and SIV-DEN. There is no similar gene in HIV-2, SIV-SMM, or other Simian Immunodeficiency Viruses (SIVs). Vpu is a 16-kd (81-amino acid) type I integral membrane protein with at least two different biological functions: (a) degradation of CD4 in the endoplasmic reticulum, and (b) enhancement of virion release from the plasma membrane of HIV-1-infected cells.

The “nef” gene encodes Nef, a multifunctional 27-kd myristylated protein produced by an ORF located at the 3′ end of the primate lentiviruses. Other forms of Nef are known, including non-myristylated variants. Nef contains PxxP motifs that bind to SH3 domains of a subset of Src kinases and are required for the enhanced growth of HIV, but not for the down-regulation of CD4.

The “vpx” gene encodes Vpx, a virion protein of 12 kD found in HIV-2, SIV-SMM, SIV-RCM, SIV-MND-2, and SIV-DRL and not in HIV-1 or other SIVs. This accessory gene is a homolog of HIV-1 vpr, and viruses with vpx carry both vpr and vpx.

“HIV genomic structural element” refers to one or more of LTR, TAR, RRE, PE, SLIP, CRS, INS sequences.

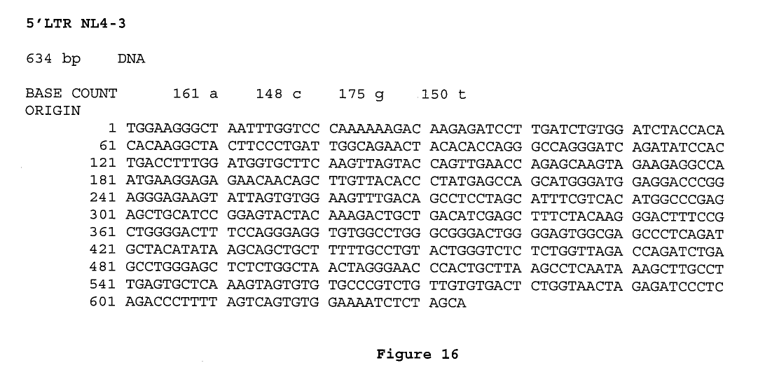

“LTR” and “long terminal repeat” refer to a DNA sequence flanking the genome of integrated proviruses. It contains important regulatory regions, especially those for transcription initiation and polyadenylation. The 5′ LTR of the reference HIV-1 strain HXB2 is exemplified by SEQ ID NO:01, FIG. 16)

“TAR” refers to a target sequence for viral trans-activation, the binding site for Tat protein and for cellular proteins. It consists of approximately the first 45 nucleotides of the viral mRNAs in HIV-1 (or the first 100 nucleotides in HIV-2 and SIV.)

“RRE” and “Rev responsive element” is an RNA element encoded within the env region of HIV-1. It consists of approximately 200 nucleotides (positions 7710 to 8061 from the start of transcription in HIV-1, spanning the border of gp120 and gp41).

“PE” and “Psi elements” refer to a set of 4 stem-loop structures preceding and overlapping the Gag start codon. PE are the sites recognized by the cysteine histidine box, a conserved motif with the canonical sequence CysX2CysX4HisX4Cys, present in the Gag p7 MC protein.

“SLIP” refers to a TTTTTT slippery site, followed by a stem-loop structure, and is responsible for regulating the −1 ribosomal frameshift out of the Gag reading frame into the Pol reading frame.

“CRS” and “cis-acting repressive sequences” refer to sequences that inhibit structural protein expression in the absence of Rev.

“INS” and “inhibitory/instability RNA sequences” refer to sequences found within the structural genes of HIV-1 and of other complex retroviruses. One of the best characterized elements spans nucleotides 414 to 631 in the gag region of HIV-1. The INS elements have been defined by functional assays as elements that inhibit expression post-transcriptionally.

2. Preliminary Data

During the development of the invention's methods and compositions, the inventor's preliminary data in Examples 1-4 herein showed that one of the benefits of the yeast-based cloning system to construct recombinant viruses (i.e., reproducing the in vivo quasispecies) was jeopardized by the need to propagate the virus in MT-4 cells for long periods of time. This creates a bottleneck that selects for viral variants more adapted to grow in vitro (4). In addition, this lengthy virus propagation step affects the commercial feasibility of the HIV-1 drug susceptibility assay by increasing its turn-around-time. To avoid the adverse effect's of the art's complementation system (i.e., co-transfection of two vectors into HEK 293T cells) on virus production from the producer cells, in one embodiment, the invention includes modifying the art's methodology to avoid the need for virus propagation, as exemplified in Example 5. This is further described below.

3. Exemplary Methods for Producing Reporter-Tagged HIV Particles

Thus, in one embodiment (summarized in Example 7 and FIG. 10), the invention provides an in vitro method for producing a replication competent chimeric HIV that contains a heterologous reporter gene, comprising a) providing 1) a first DNA sequence encoding an HIV RNA sequence (e.g., from an HIV-infected patient), 2) a first yeast vector (e.g., pRECnfl-TRPΔ(p2-INT)/URA3-hRluc vector) that lacks a second DNA sequence encoding HIV 5′ long terminal repeat (LTR) (exemplified by SEQ ID NO:01, FIG. 16), and that comprises, in operable combination, i) a third DNA sequence encoding an HIV genome sequence containing a deletion of a sequence that corresponds to the first DNA sequence, and ii) a first restriction sequence and a second restriction sequence flanking the deleted sequence that corresponds to the first DNA sequence, and 3) a second vector (e.g., eukaryotic vector pNL4-3-Δ(p24-VPR)-hRluc) that comprises, in operable combination, i) a fourth DNA sequence encoding an HIV genome sequence, wherein the HIV genome sequence comprises a heterologous sequence (e.g., linker and/or lethal gene) in place of the sequence corresponding to the first DNA sequence, and ii) a heterologous reporter gene, and 4) a host cell (e.g., mammalian HEK 293T cells), b) introducing the first DNA sequence by homologous recombination into the first yeast vector to produce a third yeast vector (e.g., pRECnfl-TRP-p2-INT) that comprises the first DNA sequence, c) contacting the third yeast vector produced in step b) with i) a first restriction enzyme that specifically cleaves the first restriction sequence, and ii) a second restriction enzyme that specifically cleaves the second restriction sequence, wherein the contacting produces a nucleotide sequence comprising the first DNA sequence, d) introducing the nucleotide sequence produced in step c) into the second vector under conditions to substitute the heterologous sequence with the first DNA sequence, thereby producing a fourth vector (e.g., pNL4-3-Δ(p24-VPR)-hRluc) that comprises, in operable combination, i) a fifth DNA sequence encoding an HIV genome sequence, wherein the HIV genome lacks a sequence corresponding to the first DNA sequence, ii) the first DNA sequence, and iii) the heterologous reporter gene, and e) transfecting the fourth vector into the host cell to produce a replication competent chimeric HIV that comprises the first DNA sequence operably linked to the heterologous reporter gene.

In one embodiment, the HIV RNA sequence is obtained from a sample. The terms “sample” and “specimen” as used herein are used in their broadest sense to include any composition that is obtained and/or derived from biological and/or environmental source, as well as sampling devices (e.g., swabs) which are brought into contact with biological and/or environmental samples. “Biological samples” include those obtained from an animal, including body fluids such as urine, blood, plasma, fecal matter, cerebrospinal fluid (CSF), semen, sputum, and saliva, as well as solid tissue. Biological samples also include a cell (such as cell lines, cells isolated from tissue whether or not the isolated cells are cultured after isolation from tissue, fixed cells such as cells fixed for histological and/or immuno-histochemical analysis), tissue (such as biopsy material), cell extract, tissue extract, and nucleic acid (e.g., DNA and RNA) isolated from a cell and/or tissue, and the like. “Environmental samples” include environmental material such as surface matter, soil, water, and industrial materials. In one preferred embodiment, the sample is from an HIV-infected subject. In other embodiments, the sample is from in vitro cultures of cells and/or HIV, from molecular clones of HIV, etc.

In one embodiment of the invention's methods, the HIV RNA sequence is reverse transcribed to DNA, followed by amplification to prepare an amplicon.

In another embodiment, the first DNA sequence encoding the HIV RNA sequence is introduced into a yeast vector by homologous recombination. “Homologous recombination” refers to a method in which nucleotide sequences are exchanged between two similar or identical strands of DNA. The process involves several steps of physical breaking and the eventual rejoining of DNA to produce new combinations of DNA sequences. In one embodiment, homologous recombination begins with a double-strand break of a first DNA sequence, and sections of DNA around the break on the 5′ end of the first DNA are removed in a process called resection. In one embodiment, recombination proceeds by strand invasion, in which an overhanging 3′ end of the first DNA sequence “invades” a second DNA sequence. A Holliday junction is formed between the first DNA sequence and second DNA sequence after strand invasion. In an alternative embodiment, recombination proceeds via a DNA repair pathway, in which a second Holliday junction forms.

Methods for using the exemplary yeast vector pRECnfl in a homologous recombination method to introduce an HIV fragment derived from a patient into the vector are described herein, and in the art (Moore et al. (2004); Dudley et al. (2009); Arts et al., Patent Application No. US 2009/0130654).

The invention's methods may optionally further comprise, prior to the transfection step, the step of transforming the fourth vector (e.g., pNL4-3-Δ(p24-VPR)-hRluc) into a bacterial cell to produce a transformed bacterial cell. This optional step may be used to amplifying the amount of DNA prior to transfection of eukaryotic host cells.

In one embodiment, the invention's methods may further comprise purifying the above-described fourth vector (e.g., pNL4-3-Δ(p24-VPR)-hRluc) from the transformed bacterial cell. Purifying may be done by positive selection using a heterologous lethal gene in the vector, to guarantee the growth only of clones carrying the patient-derived HIV sequence.

In some embodiments, the invention's methods are distinguished from those of the prior art in various respects, some of which are summarized in Table 4.

For example, in one embodiment, the invention's methods lack virus propagation in producer cells. In other words, the above described steps of homologous recombination into a yeast vector, restriction of an exemplary patient-derived HIV sequence out of the yeast vector, and subsequent ligation of the patient-derived HIV sequence into a eukaryotic vector, do not include propagation of HIV particles (that comprises a DNA sequence encoding the patient-derived HIV sequence) by a producer cell. The absence of the propagation step has the advantage of avoiding selection for viral variants that are more adapted to grow in vitro, and that have genotypic and/or phenotypic differences compared to the source patient-derived HIV.

In another distinction over the prior, in one embodiment, the invention's methods do not include co-transfection of 2 (two) vectors into a producer cell (e.g., HEK 293T) to produce infectious virions. Instead, the invention's methods, in preferred embodiments, transfect only 1 (one) vector into a producer cell to produce infectious virus particles.

In a further distinction over the prior, in one embodiment, the invention's methods do not require deleting any HIV genes from the infectious particles. Rather, in preferred embodiments, the virus particles produced by the invention's methods contain all the HIV genes, some of which being derived from a sample (e.g., from an HIV-infected patient), and the remaining genes being provided by a reference HIV (e.g., HXB2).

In some embodiments, the invention's methods further comprise step detecting the presence of the chimeric HIV that is produced by the transfection step. In one embodiment, the invention's chimeric HIV is purified. The terms “purified,” “isolated,” and grammatical equivalents thereof as used herein, refer to the reduction in the amount of at least one undesirable component (such as cell type, protein, and/or nucleic acid sequence) from a sample, including a reduction by any numerical percentage of from 5% to 100%, such as, but not limited to, from 10% to 100%, from 20% to 100%, from 30% to 100%, from 40% to 100%, from 50% to 100%, from 60% to 100%, from 70% to 100%, from 80% to 100%, and from 90% to 100%. Thus purification results in “enrichment,” i.e., an increase in the amount of a desirable cell type, protein and/or nucleic acid sequence in the sample.

In some embodiments, the second vector (e.g., eukaryotic vector pNL4-3-Δ(p24-VPR)-hRluc), into which the first DNA sequence encoding an HIV RNA sequence (e.g., from an HIV-infected patient) is introduced, comprises a fourth DNA sequence encoding an HIV genome sequence, wherein the HIV genome sequence comprises a heterologous sequence in place of the sequence corresponding to the first DNA sequence.

The heterologous sequence is exemplified by a linker sequence. “Linker sequence” when in reference to a nucleotide sequence refers to a nucleotide sequence from 5 to 200 nucleotides, including from 10 to 150, from 15 to 100, and from 20 to 100 nucleotides. The linker sequence is exemplified by the 20-nt 5′-GCATGCGGCGCGCCGTCGAC-3′ (SEQ ID NO:13) that was introduced in the pNL4-3-Δ(p24-VPR)-hRluc vector. In some embodiment, one advantage of including a linker sequence in the invention's vectors is that it reduces background expression of the deleted HIV genes. In other words, the background expression being reduced corresponds to the sequence that is cloned from the patient (e.g., p2/p7/p1/p6/PR/RT/INT). The remainder of the HIV genes could be expressed. This surprising advantage was contrary to the prior art's expectation that linker sequence may adversely affect the expression levels of adjacent genes (per Weber et al. (2006) J. Virological Methods 136:102-117, p108, 1^{st} column).

The heterologous sequence is also exemplified by a lethal gene sequence. “Lethal gene sequence” refers to a sequence whose expression by a cell brings about death of the cell. Lethal gene sequences are known in the art and exemplified by, but not limited to, the barnase gene (e.g., under control of a T7 promoter) (Flexi® Vector, Promega), Bacillus subtilis sacB gene (levansucrase) that confers sensitivity to sucrose (pDNR-LIB, Clontech), and the DNA binding domain of the mouse eukaryotic transcription factor GATA-1 (CloneSure™, PureBiotech).

The invention's methods provide several advantages, such as a) the high efficiency of cellular release and/or rapid release of the invention's reporter-tagged HIV, b) the higher success rate in producing the invention's reporter-tagged HIV when using the invention's methods that involve transfection with a single plasmid, as compared to the prior art's methods of co-transfection with two plasmids, c) the genotype of the invention's reporter-tagged HIV is the same as the genotype of the source HIV-RNA, such as from a HIV-infected patient, d) the invention's reporter-tagged HIV is replication competent, e) the replication kinetics of the invention's reporter-tagged HIV are substantially the same as the replication kinetics of its source HIV, e.g., patient-derived HIV sample, f) the invention's reporter-tagged HIV is infectious of cells that express CXCR4 and/or CCR5, g) stability of gene expression by the invention's reporter-tagged HIV over multiple rounds of replication, and h) the expression levels of HIV genes by the invention's reporter-tagged HIV are not altered when compared to the expression levels of the source HIV, e.g., patient-derived HIV genes. These advantages are further discussed below.

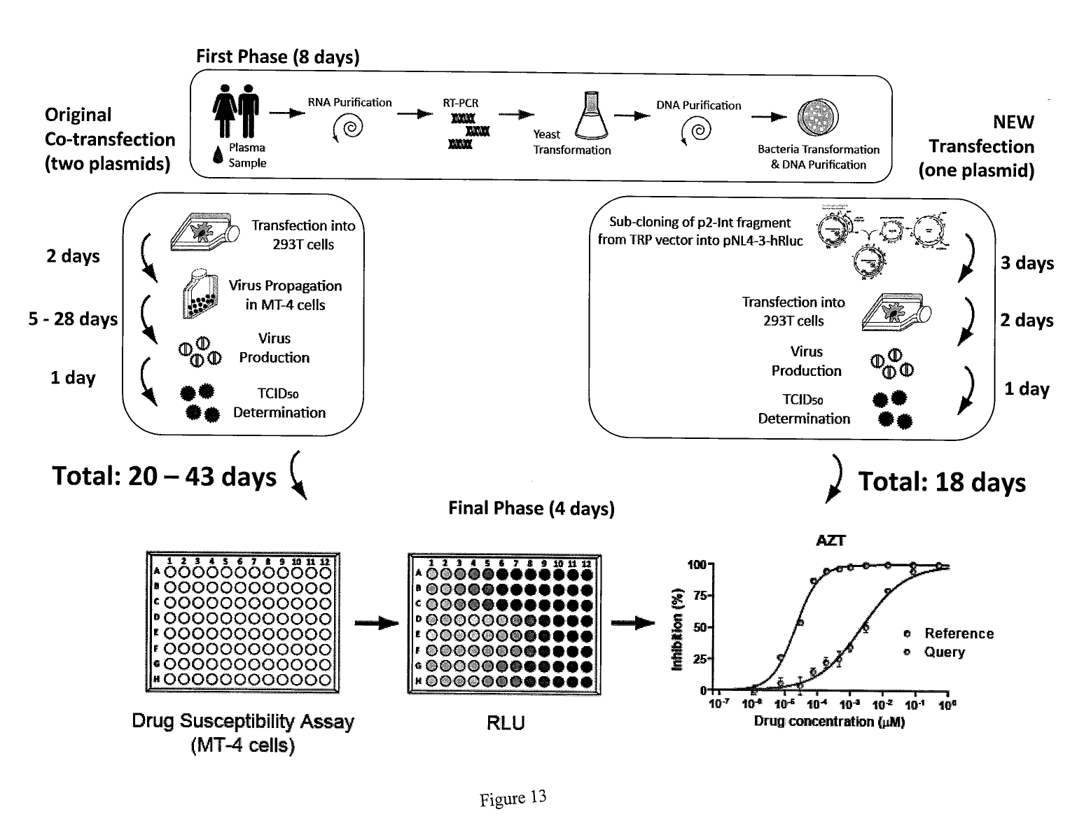

Thus, in one embodiment, one of the advantages of the invention's methods is the high efficiency of cellular release and/or rapid release of the reporter-tagged HIV. For example, the invention's reporter-tagged HIV is produced by the transfection step in less than 30 days (preferably in less than 5 days, and most preferably in less than 3 days) at TCID₅₀ equal to or greater than 10³ IU/ml, including TCID₅₀ equal to or greater than 5×10³ IU/ml, equal to or greater than 10⁴ IU/ml, equal to or greater than 5×10⁴ IU/ml, equal to or greater than 10⁵ IU/ml, equal to or greater than 5×10⁵ IU/ml, equal to or greater than 10⁶ IU/ml, equal to or greater than 5×10⁶ IU/ml, equal to or greater than 10⁷ IU/ml, etc. To illustrate, data herein in Examples 8 and 9, Table 4 and FIG. 13 show the production of the invention's reporter-tagged HIV at TCID₅₀ of from 10⁵ to 10^{6.3} IU/ml at 2 days after cell transfection. This is in contrast to the art's co-transfection methods, which produced HIV at TCID₅₀ of less than 10³ IU/ml in from 5 to 28 days after cell transfection. Also, the inventor's preliminary data in Example 2, showed that compositions and methods that are different from the preferred embodiments of the invention required from 12 to 30 days to produce HIV in MT-4 cells at TCID₅₀ equal to or greater than 10⁴ IU/ml.

Another advantages of the invention's methods is the higher success rate in producing the reporter-tagged HIV when using the invention's methods that involve transfection with a single plasmid, as compared to the prior art's methods of co-transfection with two plasmids. Thus, in one embodiment, the invention's reporter-tagged HIV is produced by the transfection step at a success rate of greater than 80% (Example 1, Table 1).

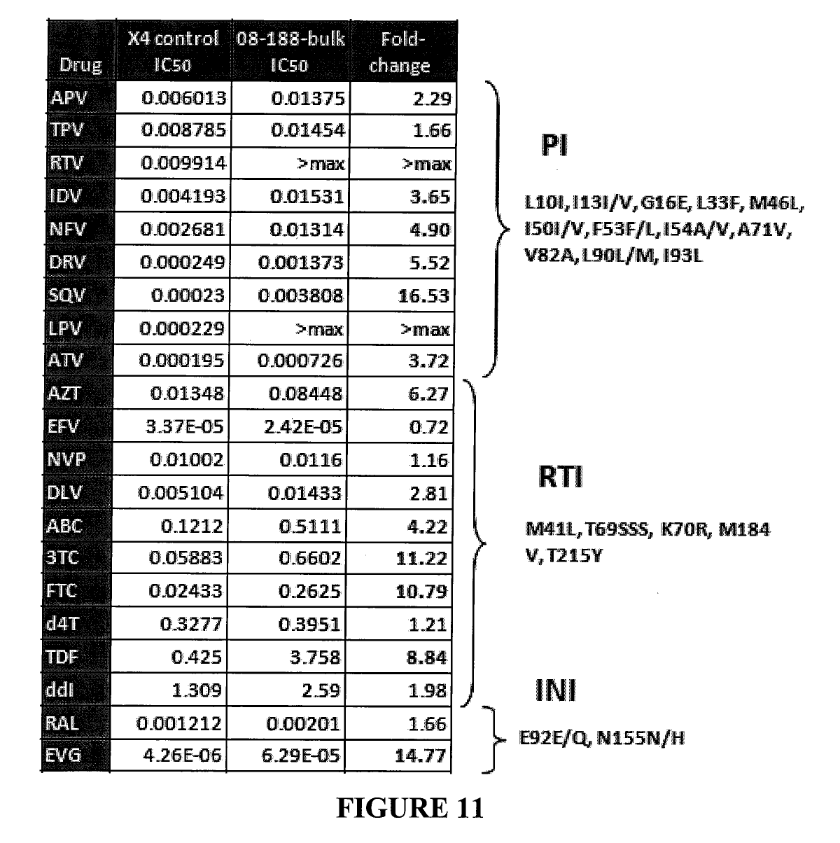

A further advantage is that the genotype of the invention's reporter-tagged HIV is the same as the genotype of the source HIV-RNA, e.g., from a HIV-infected patient. Thus, in one embodiment, the patient-derived DNA sequence that is comprised in the invention's reporter-tagged HIV, which is produced by the invention's transfection step, has from 99% to 100% identity to the source DNA sequence that encodes the HIV-infected patient's RNA sequence. For example, data herein in Example 8, FIG. 11, show that the amino acid sequence in the protease, RT, and integrase genes of the invention's virus matched the original sequence obtained from the patient's plasma sample (compare to preliminary data in Example 3).

Another advantage is that the reporter-tagged HIV produced by the invention's methods is replication competent. Thus, viral production may be monitored without interfering with the viral culture (i.e., without harvesting cells and/or supernatant for DNA/RNA purification, PCR amplification, or sequencing). Rather, viral production may be monitored by adding the luciferase substrate to viral culture and measuring the expression of firefly and/or renilla luciferase genes. In addition, the viral competition assay provides an estimate of the replicative fitness of the two viruses (query and control) that harbor the different reporter genes.

“Replication competent” virus refers to a virus that is capable of producing one or more copies of the virus following infection of a cell.

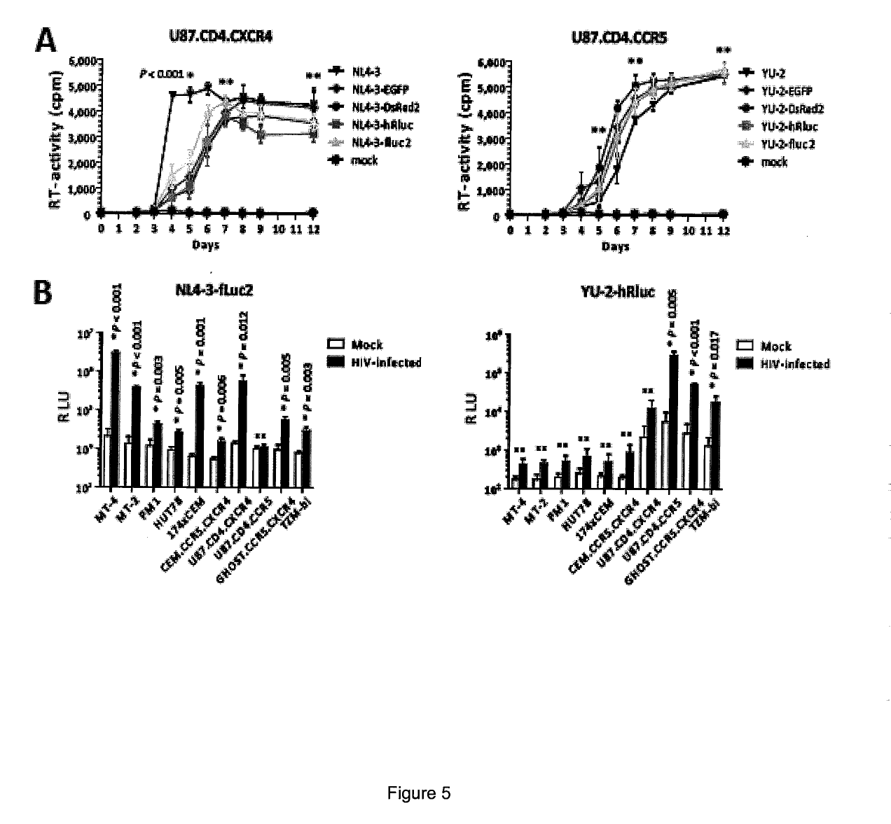

“Replication” of a virus refers to the production by a cell that is infected with the virus, of one or more copies of the virus. Replication of a virus includes the steps of adsorbing (e.g., receptor binding) to a cell, entry into a cell (such as by endocytosis), introducing its genome sequence into the cell, un-coating the viral genome, initiating transcription of the viral genome, directing expression of encapsidation proteins, and/or encapsidating the replicated viral nucleic acid sequence with the encapsidation proteins into a viral particle that is released from the cell to infect other cells. The level of replication of HIV may be determined using methods known in the art and described herein, such as by determining the level of reverse transcriptase (RT) activity (Example 5, FIG. 5A), expression of the reporter gene (Example 7 using using Dual-Glo® Luciferase Assay System (Promega)), etc. Cells suitable for such determination include, without limitation, human T cells, MT4, MT2, Jurkat, PM1, human cervical epithelial carcinoma cells (TZM-bl), human astroglioma cells (U87.CD4.CXCR4) (FIG. 5 & Weber et al. (2006)).

Yet another advantage is that the replication kinetics of the invention's reporter-tagged HIV is substantially the same as the replication kinetics of its source, patient-derived HIV sample. “Replication kinetics” refers to the change in the number of virus particles produced by a cell over a period of time, such as from 1 to 21 days after infection, including from 1 to 12 days after infection. Data herein show that the replication kinetics of the invention's hRluc expressing HIV and fluc2-expressing HIV are substantially the same over a period from 1 to 12 days as the replication kinetics of the source, patient derived HIV (Example 5, FIG. 5A). Also, the data show that the invention's hRluc-expressing HIV that were obtained only 48 hours post-transfection also carried the renilla luciferase (hRluc) gene without a notable effect in viral replication (Example 9).

A further advantage is that the invention's reporter-tagged HIV is infectious of cells that express CXCR4 and/or CCR5. The terms “infectious,” “infectivity,” and “infection” when in reference to HIV interchangeably refer to the ability of HIV to fuse with a target cell to gain entry and/or replicate and/or transcribe its genes and/or assemble viral particles and/or release viral particles. Infectivity may be determined, directly or indirectly, by any method, such as by in vitro cell-cell fusion assays using the exemplary HeLa-P5L and HeLa-ADA cell lines, by in vitro HIV infection assays using peripheral blood mononuclear cells (PMBC), and by in vivo HIV infection assays in animals, such as the art's humanized mouse model and macaque model. Infectivity may be expressed as a tissue culture dose for 50% infectivity (“TCID₅₀”) and expressed as infectious units per milliliter (IU/ml), as disclosed herein. Data herein in FIG. 5B demonstrate that the invention's hRluc-tagged HIV and fluc2-tagged HIV were able to infect one or more of the following exemplary cells that express the receptor CXCR4 and/or CCR5: MT-4, MT-2, PM1, HUT78, 174xCEM, CEM.CCR5.CXCR4, U87.CD4.CXCR4, U87.CD4.CCR5, GHOSTX4/R5, and TZM-bl.

Yet another advantage is the stability of gene expression by the invention's reporter-tagged HIV (as exemplified by expression of the reporter gene) over multiple rounds of replication. In one embodiment, the level of expression of the DNA sequence that encodes the exemplary patient-derived HIV RNA, and that is comprised in the invention's reporter-tagged HIV, is substantially the same for at least 5 days (preferably for at least 10 days, at least 15 days, at least 20 days, at least 25 days, at least 30 days, at least 35 days, and/or at least 40 days) following the transfection of the vector (e.g., pNL4-3-Δ(p24-VPR)-hRluc) into the host cell (e.g., mammalian cell).

For example, in one embodiment, the stability of HIV gene expression by the invention's reporter-tagged HIV was determined using a phenotypic approach, i.e., by quantifying the ratio of virus production and expression of the reporter gene, instead of using a genotypic approach, i.e., by quantifying copies of the HIV and reporter genes. Using this phenotypic approach, the inventors observed that the expression of the renilla (hRluc) gene by the virus was substantially unaltered for about 32 days, before observing a decrease in the expression of this gene. Expression of the firefly gene, which is larger than hRluc and EGFP or DsRed2, began to decrease after about 2 weeks. These prolonged periods of stable expression allow successful completion of drug susceptibility tests in about 3 to 4 days.

A further advantage is that the expression levels of HIV genes by the invention's reporter-tagged HIV are not altered when compared to the expression levels of the source, patient-derived HIV genes. In one embodiment, the expression level of one or more HIV genes by the invention's reporter-tagged HIV is substantially the same as the expression level of the the corresponding HIV gene in the source sample, e.g, HIV-infected patient sample. In a particular embodiment, the HIV gene is gag, pol, env, tat, rev, vif, vpr, vpu, nef, and/or vpx. In a preferred embodiment, the exemplary HIV RNA sequence that was used to construct the invention's vectors included a sequence spanning the 3′Gag (p2/p7/p1/p6), protease, reverse transcriptase and the integrase genes (Example 7).

4. Reporter Genes

In some embodiments, the vector that is used for homologous recombination (e.g., the yeast vector pRECnfl-TRPΔ(p2-INT)/URA3-hRluc vector), and/or the vector used for transfection (e.g., the eukaryotic vector pNL4-3-Δ(p24-VPR)-hRluc) comprises a heterologous reporter gene.

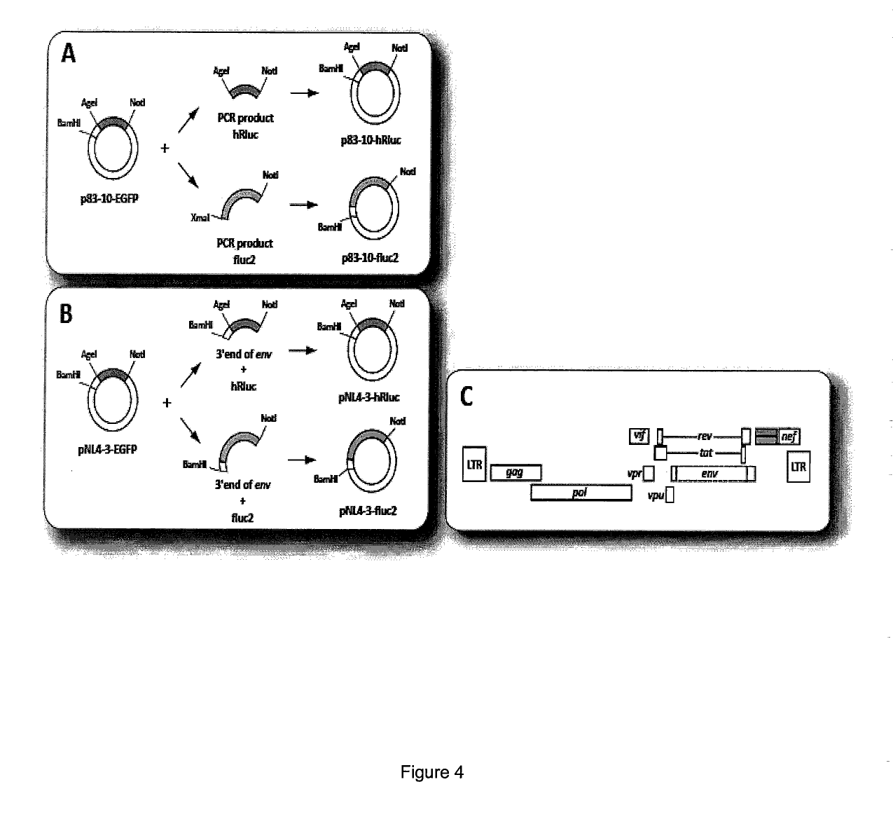

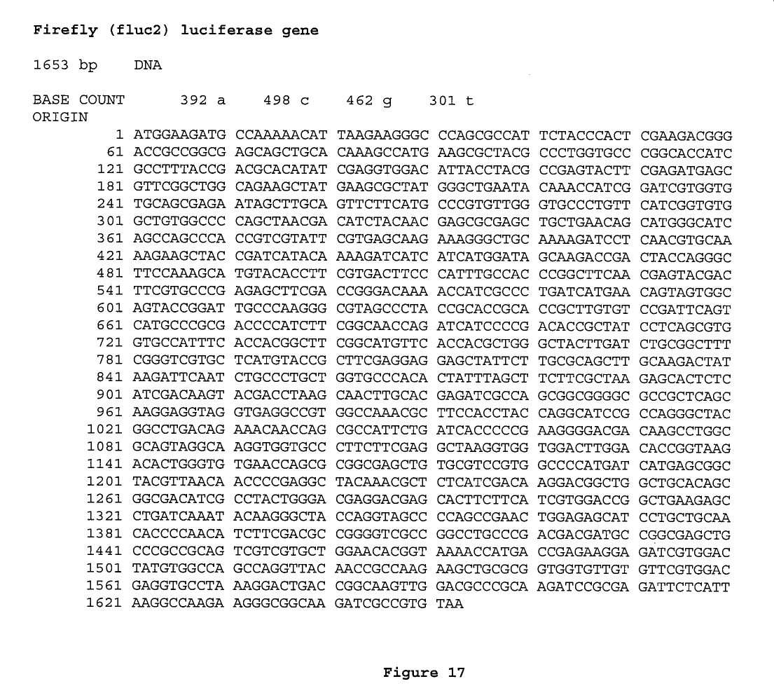

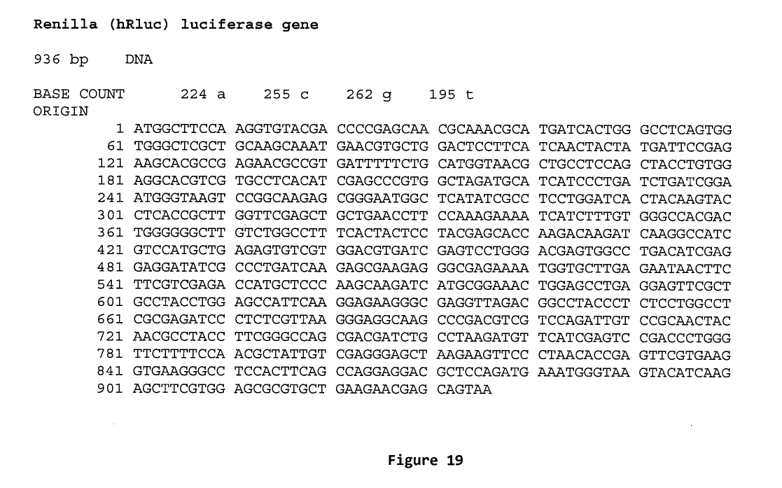

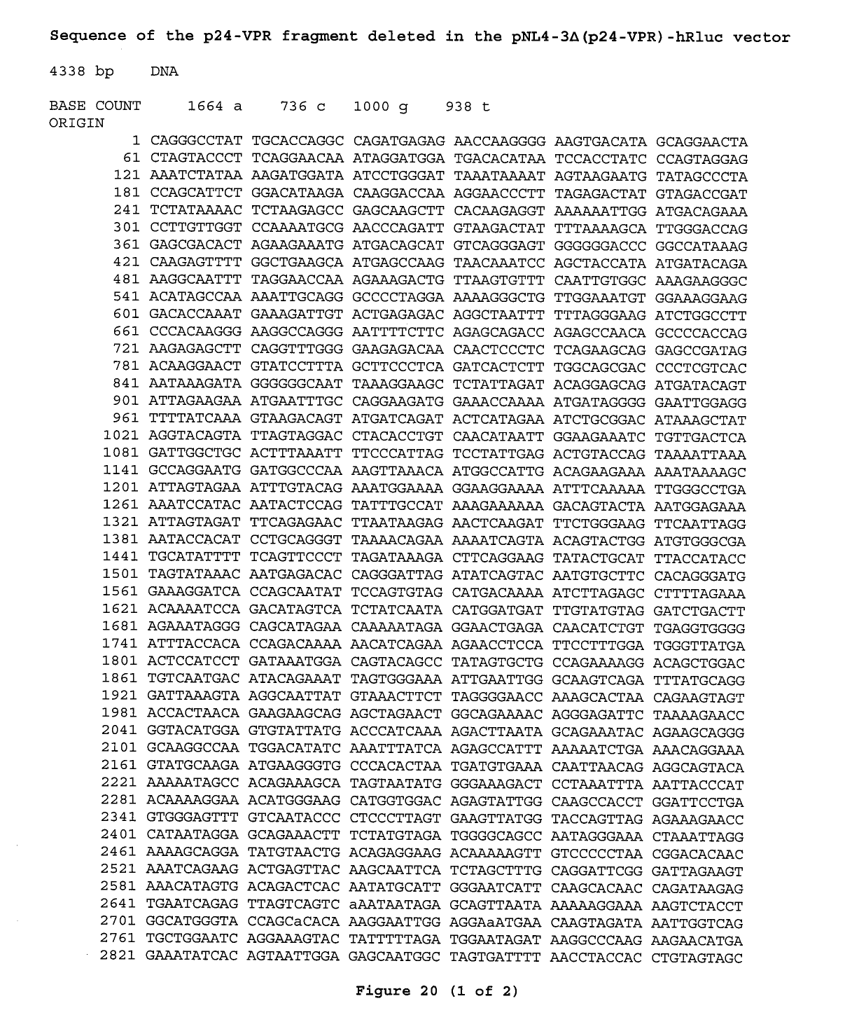

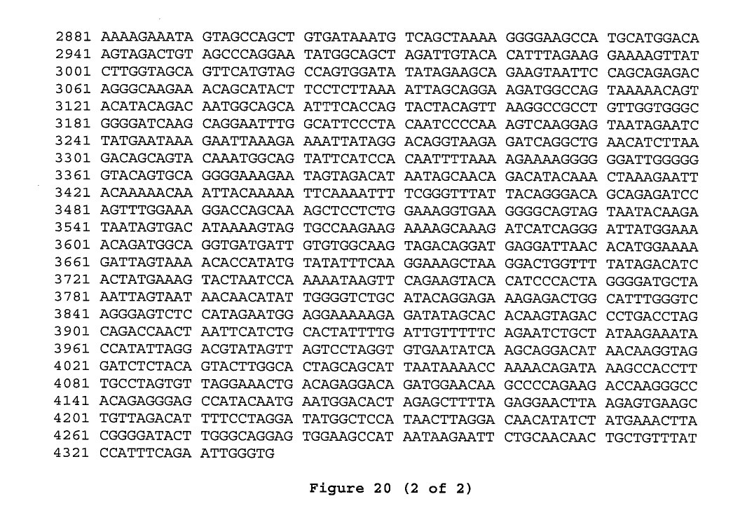

“Reporter sequence” and “marker sequence” are used interchangeably to refer to DNA, RNA, and/or polypeptide sequences that are detectable in any detection system, including, but not limited to enzyme (e.g., ELISA, as well as enzyme-based histochemical assays), fluorescent, radioactive, and luminescent systems. Exemplary reporter gene sequences include, for example, β-glucuronidase gene, green fluorescent protein (GFP) gene, E. coli β-galactosidase (LacZ) gene, Halobacterium β-galactosidase gene, E. coli luciferase gene, Neurospora tyrosinase gene, Aequorin (jellyfish bioluminescenece) gene, human placental alkaline phosphatase gene, and chloramphenicol acetyltransferase (CAT) gene. Reporter gene may be monitored by fluorescence microscopy, flow cytometry, etc. It is not intended that the present invention be limited to any particular reporter sequence. In one embodiment, the reporter sequence comprises one or more of firefly luciferase gene (fluc2) of FIGS. 4 and 5, exemplified by SEQ ID NO:02 of FIG. 17; renilla luciferase gene (hRluc) of FIGS. 4 and 5, exemplified by SEQ ID NO:4 of FIG. 19; enhanced green fluorescent protein (EGFP) of FIG. 5; red Discosoma sp. red fluorescent (DsRed2) protein of FIG. 5; enhanced yellow fluorescent protein (YFP) (Levy et al. (2004) PNAS 101:4204-4209); cyan fluorescent protein (CFP): (Levy et al. (2004)).

5. Vectors

The invention contemplates the use of vectors in the inventor's methods to produce chimeric HIV. The terms “vector” and “vehicle” are used interchangeably in reference to nucleic acid molecules that transfer DNA segment(s) from one cell to another. Vectors are exemplified by, but not limited to, plasmids, linear DNA, encapsidated virus, etc. that may be used for expression of a desired sequence. Vectors include expression vectors. An “expression vector” refers to a recombinant DNA molecule containing a desired coding sequence and appropriate nucleic acid sequences necessary for the expression (i.e., transcription and/or translation) of the operably linked coding sequence in a particular host cell. Expression vectors are exemplified by, but not limited to, plasmid (including “bacterial artificial chromosomes,” phagemid, shuttle vector, cosmid, virus, chromosome, mitochondrial DNA, and nucleic acid fragment. Expression vectors include “eukaryotic vectors,” i.e., vectors that are capable of replicating in a eukaryotic cell (e.g., insect cells, yeast cell, mammalian cells, etc.) and “prokaryotic vectors,” i.e., vectors that are capable of replicating in a prokaryotic cell (e.g., E. coli). Thus, a eukaryotic vectors includes a “yeast vector,” i.e., a vector that is capable of replication in a yeast cell. Nucleic acid sequences used for expression in prokaryotes include a promoter, optionally an operator sequence, a ribosome binding site and possibly other sequences. Eukaryotic cells are known to utilize promoters, enhancers, and termination and polyadenylation signals.

Vectors (i.e., plasmids, linear DNA, encapsidated virus, etc.) may be introduced into cells using techniques well known in the art and disclosed herein. The term “introducing” a nucleic acid sequence into a cell refers to the introduction of the nucleic acid sequence into a target cell to produce a “transformed,” “transfected,” and/or “transgenic” cell. Methods of introducing nucleic acid sequences into cells are well known in the art and disclosed herein. For example, where the nucleic acid sequence is a plasmid or naked piece of linear DNA, the sequence may be “transfected” into the cell using, for example, calcium phosphate-DNA co-precipitation, DEAE-dextran-mediated transfection, polybrene-mediated transfection, electroporation, microinjection, liposome fusion, lipofection, protoplast fusion, and biolistics. Alternatively, where the nucleic acid sequence is encapsidated into a viral particle, the sequence may be introduced into a cell by “infecting” the cell with the virus.

Transformation of a cell may be stable or transient. The terms “transient transformation” and “transiently transformed” refer to the introduction of one or more nucleotide sequences of interest into a cell in the absence of integration of the nucleotide sequence of interest into the host cell's genome. Transient transformation may be detected by, for example, enzyme-linked immunosorbent assay (ELISA) that detects the presence of a polypeptide encoded by one or more of the nucleotide sequences of interest. Alternatively, transient transformation may be detected by detecting the activity of the protein encoded by the nucleotide sequence of interest. The term “transient transformant” refer to a cell that has transiently incorporated one or more nucleotide sequences of interest.

In contrast, the terms “stable transformation” and “stably transformed” refer to the introduction and integration of one or more nucleotide sequence of interest into the genome of a cell. Thus, a “stable transformant” is distinguished from a transient transformant in that, whereas genomic DNA from the stable transformant contains one or more heterologous nucleotide sequences of interest, genomic DNA from the transient transformant does not contain the heterologous nucleotide sequence of interest. Stable transformation of a cell may be detected by Southern blot hybridization of genomic DNA of the cell with nucleic acid sequences that are capable of binding to one or more of the nucleotide sequences of interest. Alternatively, stable transformation of a cell may also be detected by the polymerase chain reaction of genomic DNA of the cell to amplify the nucleotide sequence of interest.

“Gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process.

Large numbers of suitable expression vectors that function is prokaryotic, eukaryotic cells, and insect cells are known to those of skill in the art, and are commercially available. Prokaryotic bacterial expression vectors are exemplified by pBR322, pUC, pYeDP60, pQE70, pQE60, pQE-9 (Qiagen), pBS, pD10, phagescript, psiX174, pbluescript SK, pBSKS, pNH8A, pNH16a, pNH18A, pNH46A (Stratagene); ptrc99a, pKK223-3, pKK233-3, pDR540, pRIT5 (Pharmacia). Eukaryotic expression vectors are exemplified by pMLBART, pSV2CAT, pOG44, PXT1, pSG (Stratagene) pSVK3, pBPV, pMSG, pSVL (Pharmacia), pGEMTeasy plasmid, pCambia1302 (for plant cell transformation using the exemplary Agrobacteria tumefaciens strain GV3101), and transcription-translation (TNT®) coupled wheat germ extract systems (Promega). Baculovirus expression vectors for expression in insect cells are also commercially available (e.g., Invitrogen). Any other expression vector may be used as long as it is replicable in the host cell.

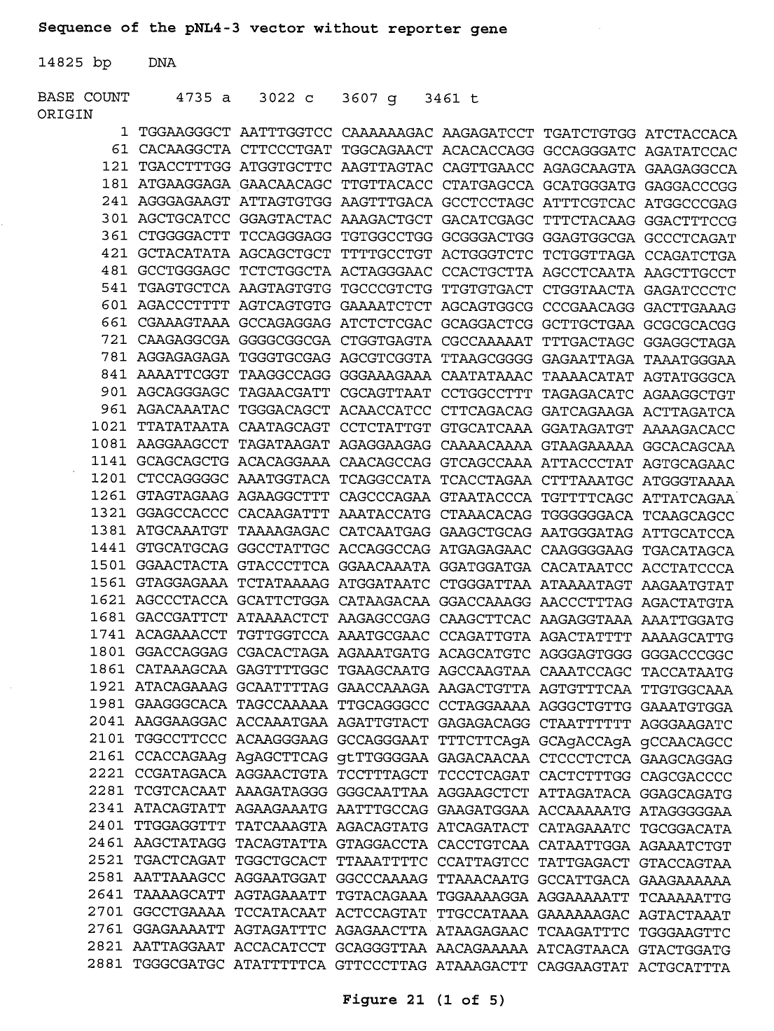

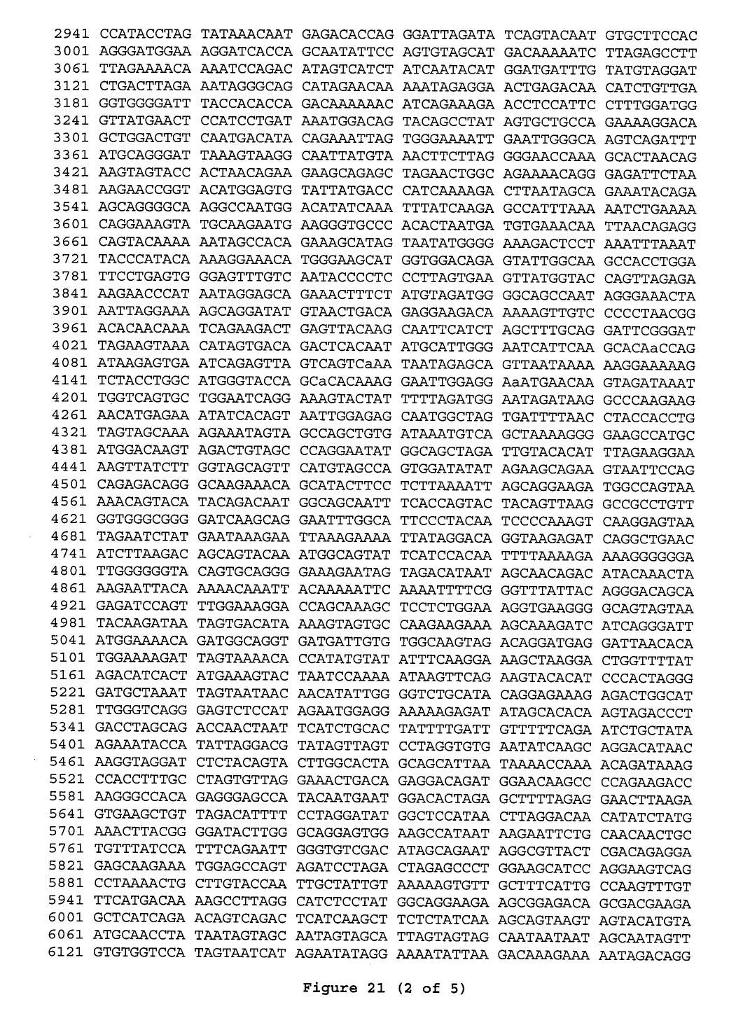

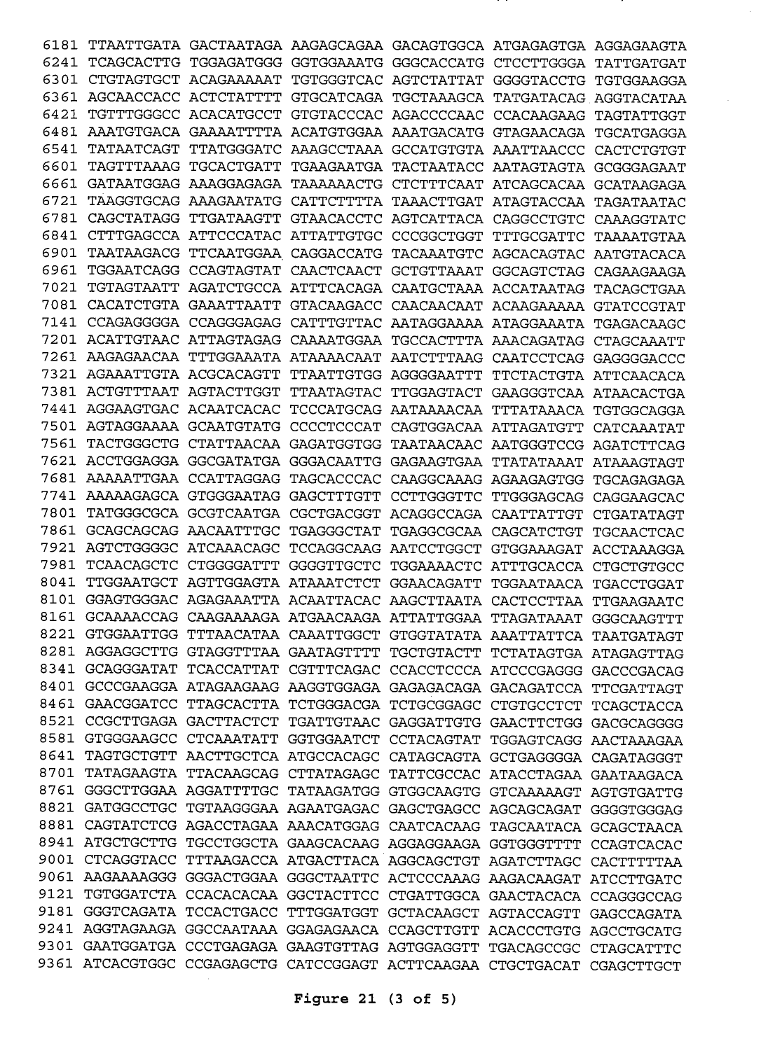

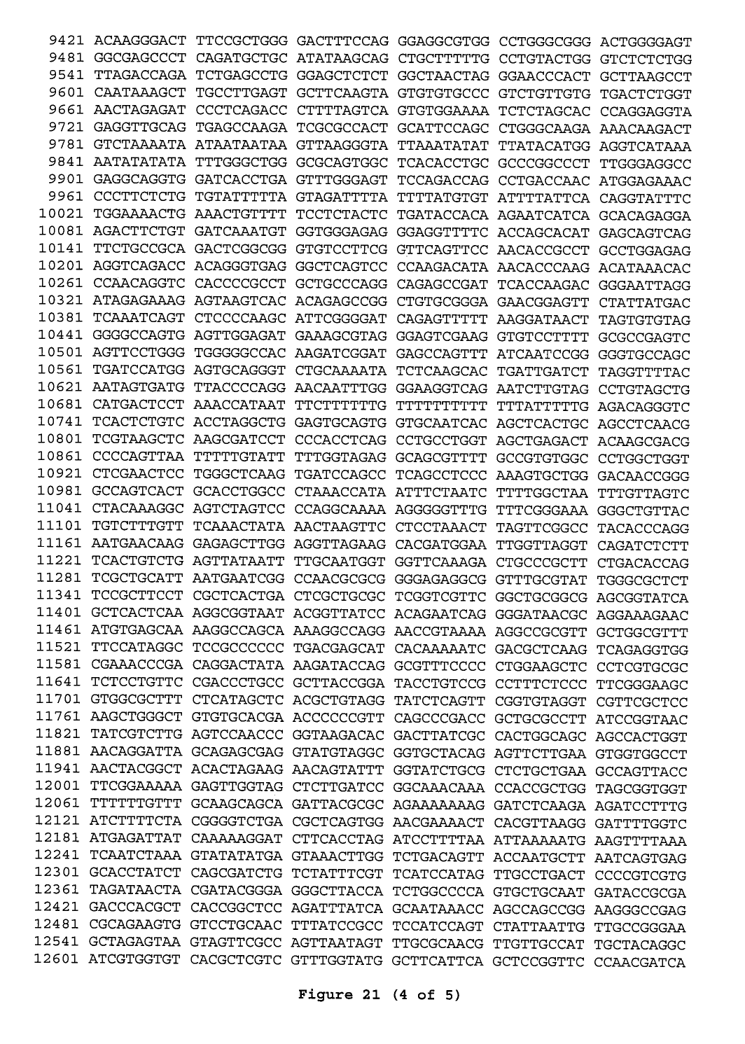

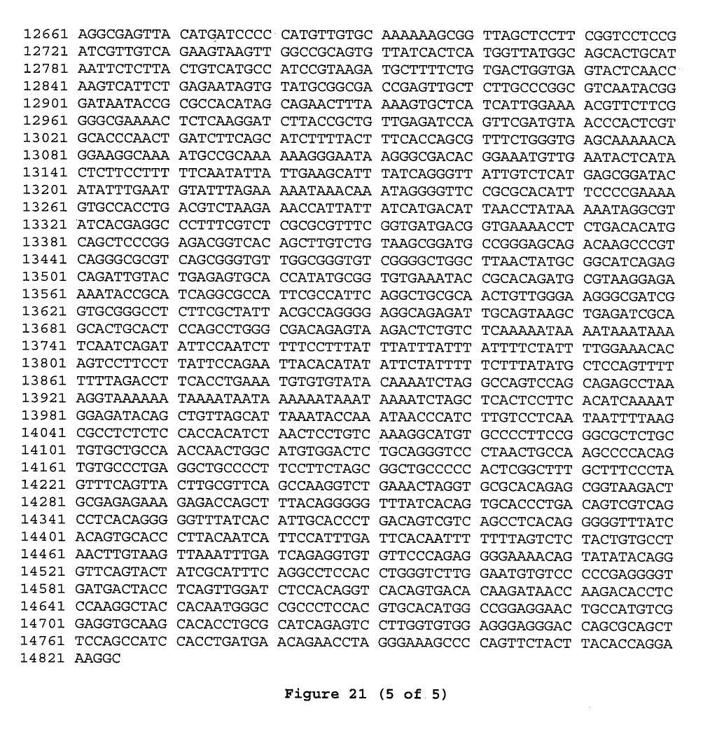

In one preferred embodiment, the expression vector is a yeast vector, exemplified by pRECnfl and derivatives thereof (Moore et al. (2004)) in “Methods in Molecular Biology, vol 304, pp. 371-387, Edited by t. Zhu, Humana Press Inc. Totowa, N.J.; Dudley et al. (2009) BioTechniques 46(6):297-305; Arts et al., Patent Application No. US 2009/0130654). In another embodiment, the expression vector is a mammalian vector, exemplified by the pUC-based pNL4-3 plasmid (SEQ ID NO:06 of FIG. 21) and derivatives thereof, including pCHUS (Abad et al. (2002) Int Conf AIDS, 14:Abstract No. MoPeB3126.

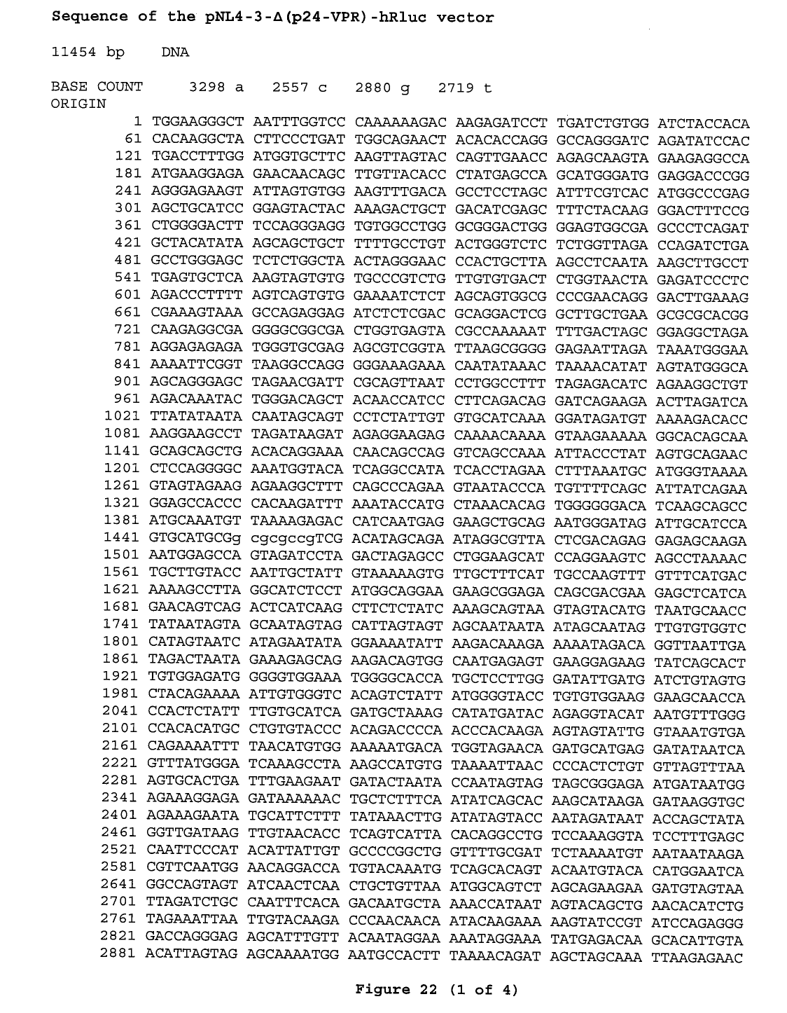

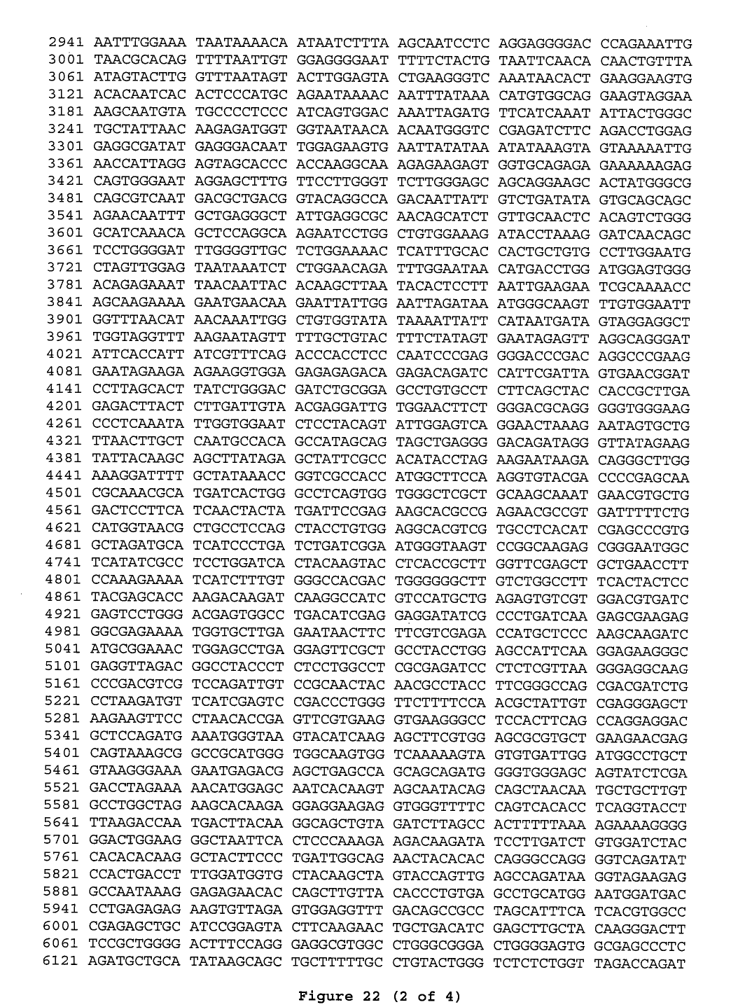

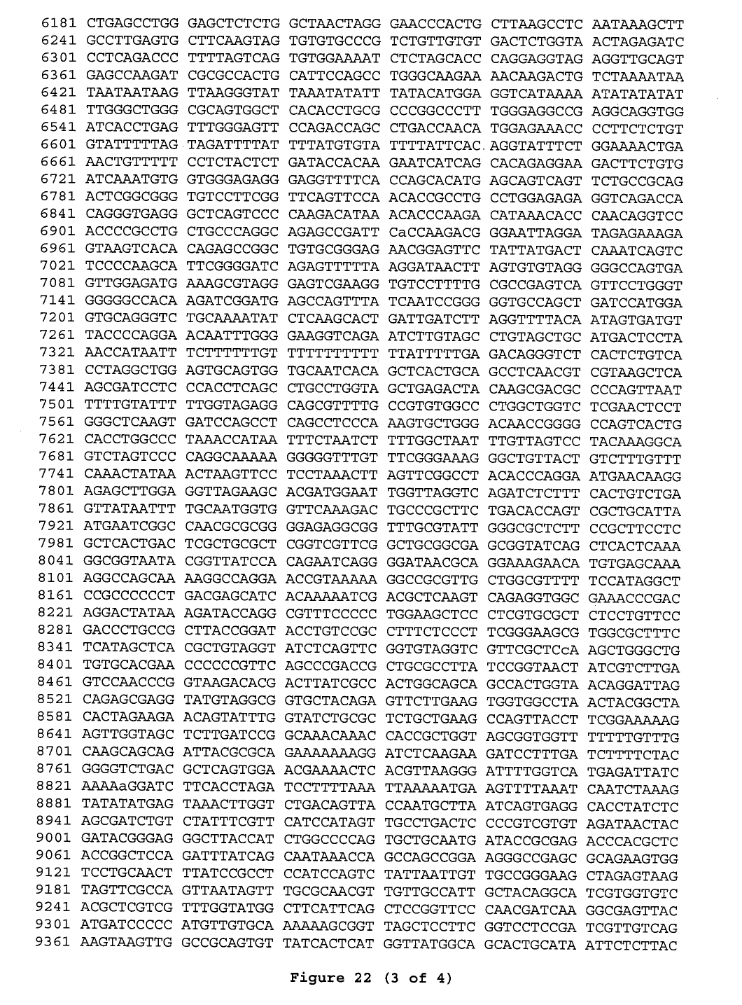

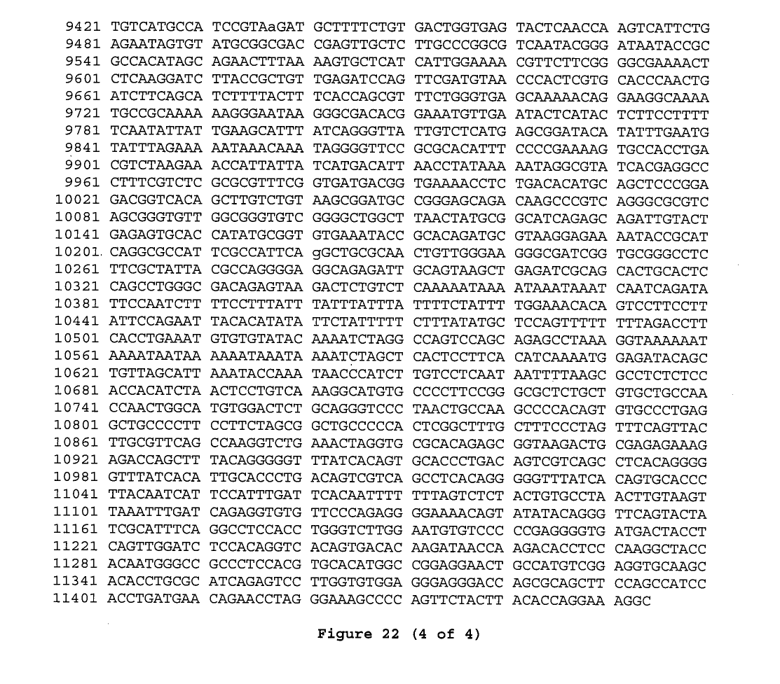

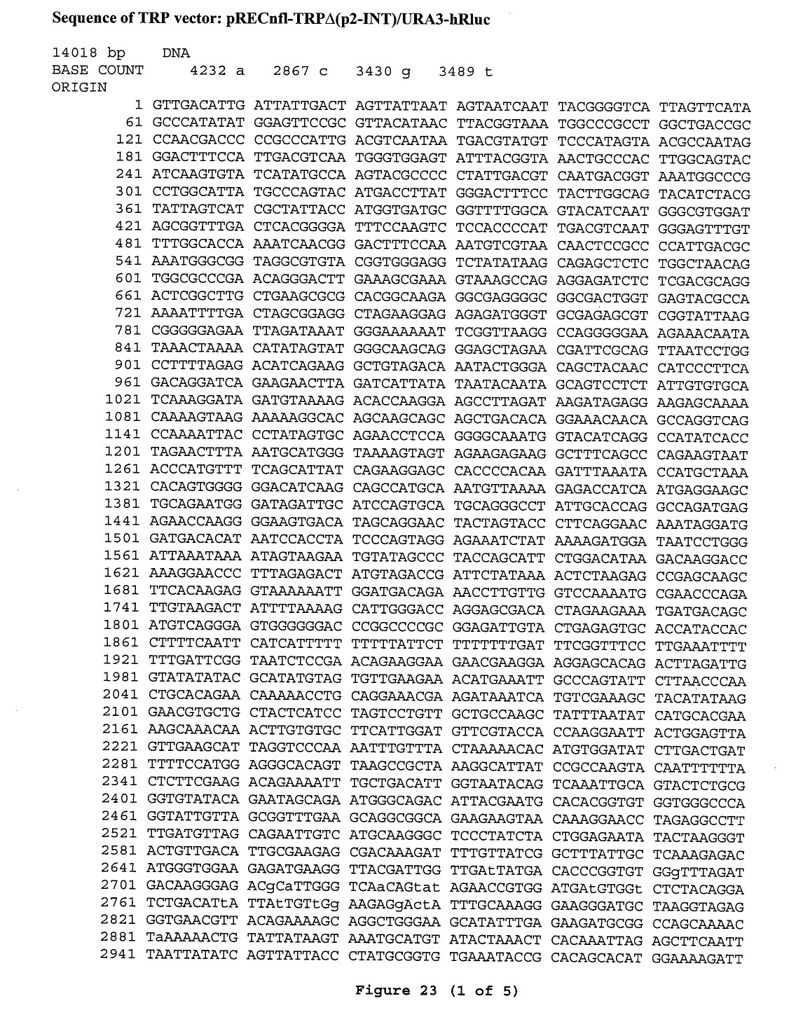

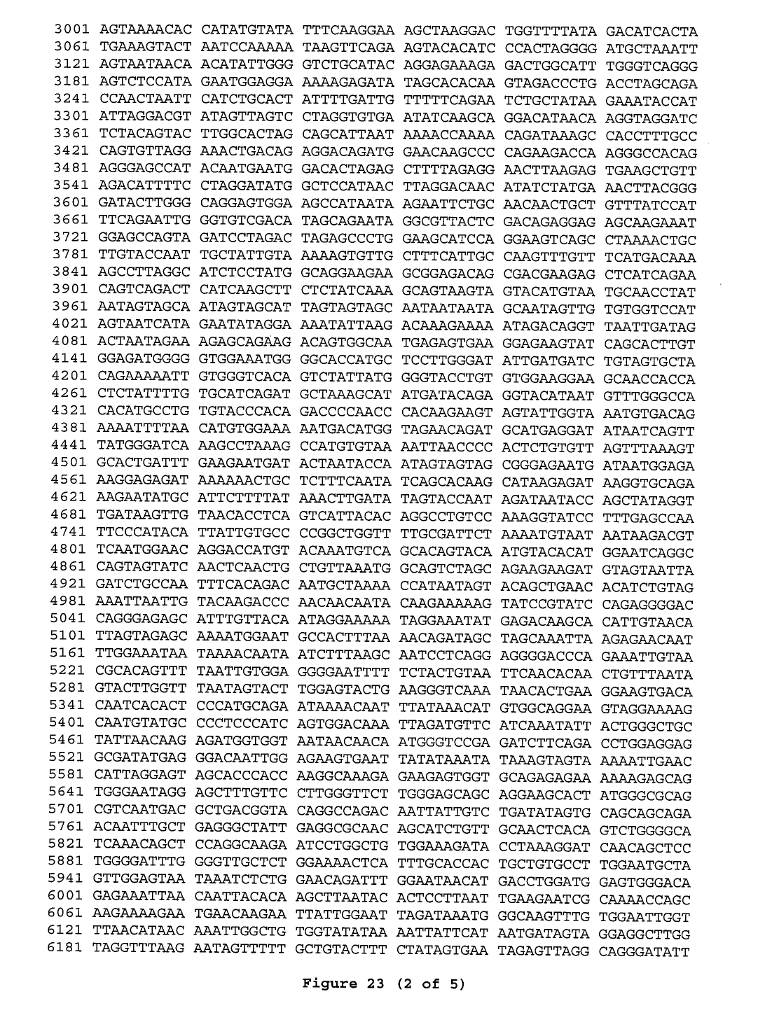

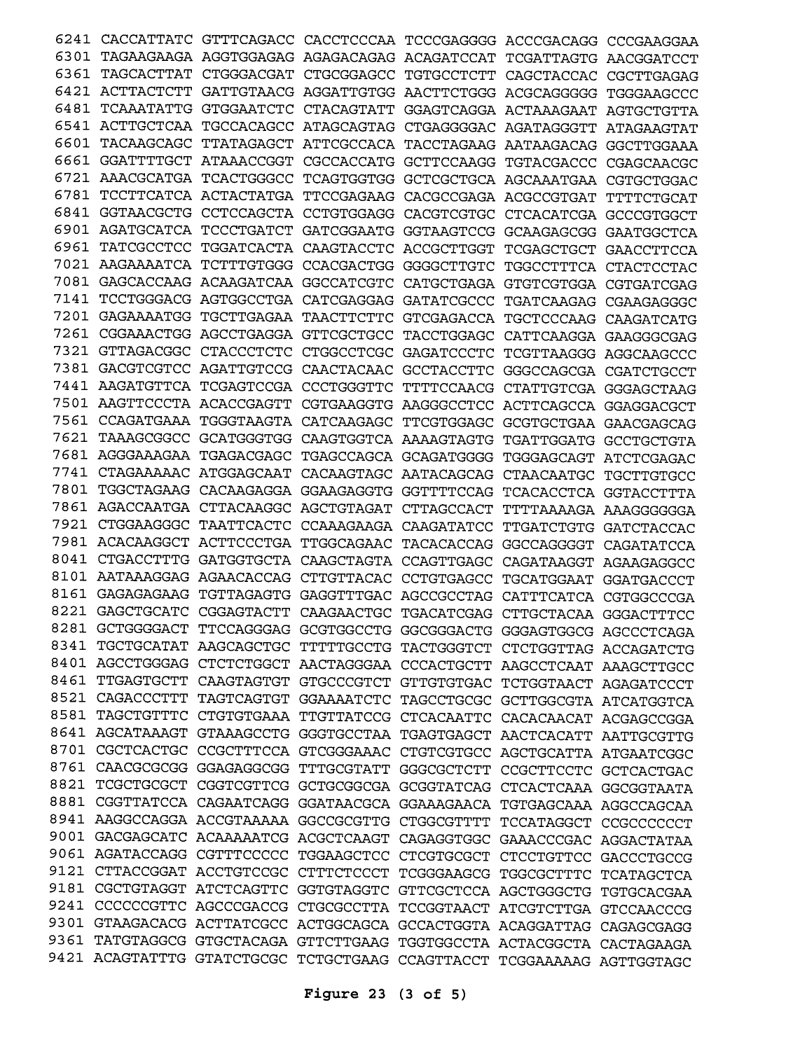

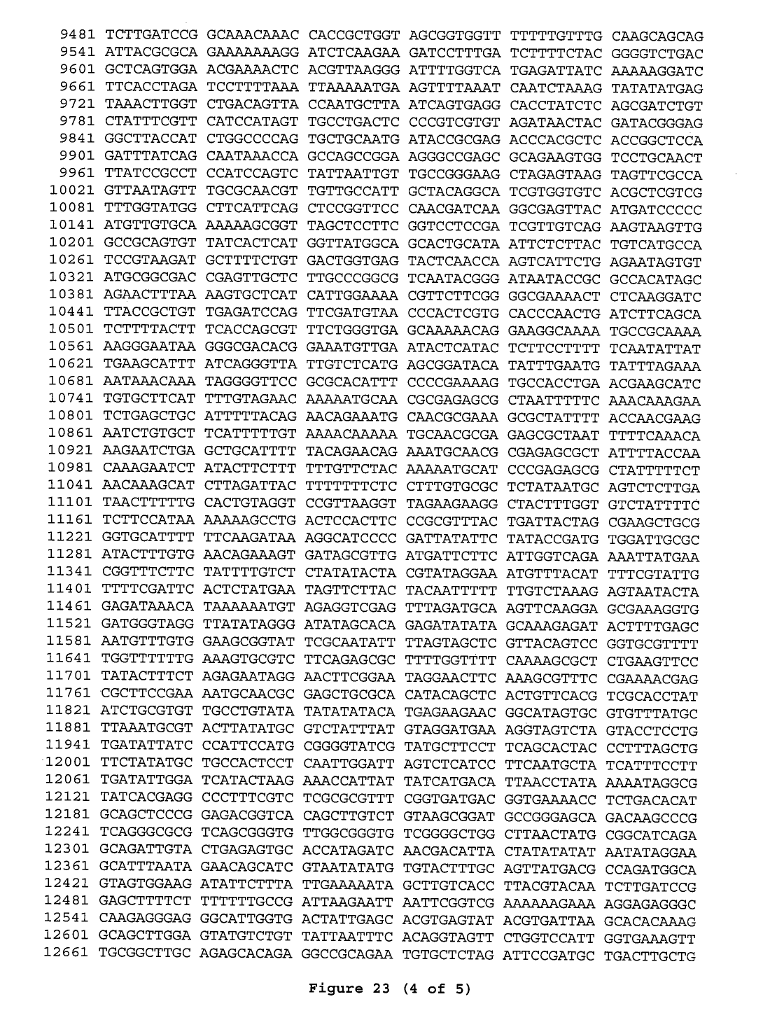

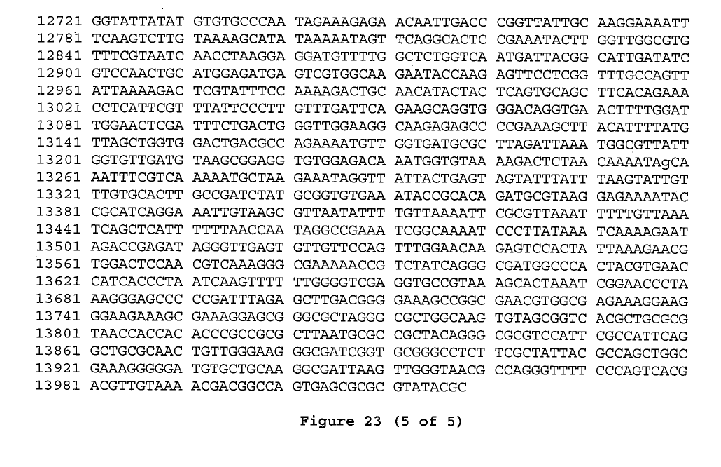

Some of the exemplary vectors generated in the invention's methods include, without limitation, the yeast vectors pRECnfl-TRPΔ(p2-INT)/URA3-hRluc, and pRECnfl-TRPΔ(p2-INT)/URA3-hRluc (SEQ ID NO:08 of FIG. 23) (FIG. 10). The invention further provides the eukaryotic vector pNL4-3-Δ(p24-VPR)-hRluc (SEQ ID NO:07 of FIG. 22), which is also referred to a pNL4-3-Δ(SphI-SalI)-hRluc in FIGS. 9 and 10.

In particular, the invention contemplates a composition comprising a yeast vector that lacks a DNA sequence encoding HIV 5′ long terminal repeat (LTR) (exemplified by SEQ ID NO:01 of FIG. 16) and that comprises, in operable combination, i) a first DNA sequence encoding an HIV genome sequence containing a deletion of an HIV sequence, and ii) a first restriction sequence and a second restriction sequence flanking the deleted HIV sequence. While a reporter gene is not necessary, in some embodiments, the vector further comprises iii) a reporter gene. In a particular embodiment the vector comprises pRECnfl-TRPΔ(p2-INT)/URA3-hRluc (SEQ ID NO:08 of FIG. 23, also shown in FIG. 10, step 2). In a more preferred embodiment, the deleted HIV sequence is substituted with a corresponding sequence, e.g., from a HIV-infected subject.

In addition, the invention contemplates a composition comprising a vector that comprises, in operable combination, i) a DNA sequence encoding an HIV genome sequence containing a deletion of an HIV sequence, wherein the deleted HIV sequence is substituted by a heterologous sequence (e.g., linker and/or lethal gene), and ii) a reporter gene. In some embodiments, the vector further comprises iii) a first restriction sequence and a second restriction sequence that flank the heterologous sequence. In a preferred embodiment, the vector comprises pNL4-3-Δ(p24-VPR)-hRluc SEQ ID NO:07 of FIG. 22, which is also referred to a pNL4-3-Δ(SphI-SalI)-hRluc in FIG. 9 and FIG. 10, step 3. In a particular embodiment, the deleted HIV sequence is substituted with a corresponding sequence from a HIV-infected subject.

6. Restriction Sequences

In some preferred embodiments, the DNA sequence that encodes HIV RNA (e.g., from a HIV-infected patient) is inserted into a first vector (e.g., a yeast vector such as pRECnfl-TRPΔ(p2-INT)/URA3-hRluc) such that it is flanked by a first restriction sequence and a second restriction sequence. In a more preferred embodiment, the first restriction sequence and the second restriction sequence are different, such as SphI and SalI restriction sequences.

In a subsequent step, the DNA sequence that encodes HIV RNA from the exemplary HIV-infected patient is used to replace a heterologous sequence (e.g., linker and/or lethal gene) in a vector (e.g., a eukaryotic vector such as pNL4-3-Δ(p24-VPR)-hRluc). To facilitate this, the heterologous sequence is flanked by the same first restriction sequence and the second restriction sequence that flank the DNA sequence in the first vector.

“Restriction enzyme” refers to an enzyme that specifically binds to a particular nucleotide sequence, referred to as a “binding sequence” of double-stranded DNA (dsDNA) molecule, and whose binding results in cleavage of the DNA molecule at a restriction site between two nucleotides. Restriction sites may be located within the restriction enzyme binding sequence (e.g., the restriction sites for EcoRV, EcoRI, SmaI, HindIII, PacI, and NotI). Alternatively, restriction sites may be located substantially adjacent to the restriction enzyme binding sequence (e.g., the restriction sites for BseRI, BsgI, BsmBI, FokI, and SapI).

In one embodiment, the SphI restriction site 5′-GCATGC-3′ (SEQ ID NO:10) and the SalI restriction site 5′-GTCGAC-3′ (SEQ ID NO:11) were used to clone a patient's HIV-1 p24-VPR fragment into pNL4-3, and an AscI restriction site 5′-GGCGCGCC-3′ (SEQ ID NO:12) was used to linearize the vector.

The invention is not limited to the exemplary restriction sites and/or enzymes disclosed herein. Thus, in one embodiment, the invention's vectors may be designed to contain unique restriction sites for insertion of nucleotide sequences, linearizing plasmids, etc.

In one embodiment, the restriction sites that flank the HIV sequence that is deleted from the first vector (e.g., yeast vector pRECnfl-TRPΔ(p2-INT)/URA3-hRluc) that lacks the HIV 5′ long terminal repeat (LTR), are not used to clone and produce virus, but to introduce patient-derived HIV sequences into a second plasmid.

7. Phenotyping and Genotyping

The invention's compositions and methods are useful for determining the phenotypic susceptibility of HIV to at least one test compound. Thus, in one embodiment, the methods may further comprise contacting the invention's reporter-tagged HIV with a test compound, and optionally further comprise determining the phenotypic susceptibility of the HIV to the test compound. In some embodiments, it may be desirable to include in the invention's method the step of generating a database that comprises the phenotypic susceptibility of the HIV to the test compound. The database may be generated manually, and preferably by a computer system.

“Test compound” refers to any compound of interest to one skilled in the art (e.g., naturally occurring, synthetic, organic, inorganic, polypeptide sequence, nucleic acid sequence, small molecule, non-peptide, antibody, etc.), and includes anti-HIV drugs (i.e., compounds that are known or suspected of targeting any stage of the HIV life cycle and/or any of the enzymes essential for HIV replication and/or survival). Amongst the anti-HIV drugs that have been approved for AIDS therapy are nucleoside reverse transcriptase inhibitors (“NRTIs”) such as AZT, ddl, ddC, d4T, 3TC, and abacavir; nucleotide reverse transcriptase inhibitors such as tenofovir; non-nucleoside reverse transcriptase inhibitors (“NNRTIs”) such as nevirapine, efavirenz, delavirdine, and etravirine; protease inhibitors (“PIs”) such as darunavir, saquinavir, ritonavir, indinavir, nelfinavir, amprenavir, lopinavir and atazanavir; fusion inhibitors, such as enfuvirtide, co-receptor antagonists such as maraviroc and integrase inhibitors such as raltegravir. Some of the anti-HIV drugs are listed in FIG. 11.

“Phenotypic susceptibility” of a virus to a test compound refers to a drug concentration that produces a particular level of reduction in the level of virus replication when compared to a reference. In one embodiment, phenotypic susceptibility may be expressed as a change in the level of infectivity of the virus, compared to a wild type virus, in the presence of the test compound, such as by using EC₅₀ and/or EC₉₀ values (the EC₅₀ and EC₉₀ value being the drug concentration that inhibits replication of 50% and 90%, respectively, of the viral population). Hence, susceptibility of a virus towards a test compound can be expressed as a fold change in susceptibility, wherein the fold change is derived from the ratio of, for instance the EC₅₀ values of a mutant virus compared to the EC₅₀ values of a wild type virus. In particular, the susceptibility of a mutant virus may also be expressed as resistance of the mutant virus, wherein the result is indicated as a fold change in EC₅₀ of the mutant virus as compared to the EC₅₀ of the wild type virus.

In another embodiment, phenotypic susceptibility of a virus to a test compound may be expressed as a change in the level of infectivity (such as the level of 50% infectivity (“TCID₅₀”)) of the virus in the presence of the test compound compared to in the absence of the test compound, as disclosed herein.

In some embodiments, the susceptibility of a virus to a drug is tested by determining the cytopathogenicity of the virus to cells and/or by determining the replicative capacity of the virus in the presence of at least one test compound, relative to a wild type or reference virus.

In yet another embodiment, phenotypic susceptibility of a virus to a test compound may be derived from database analysis such as the VirtualPhenotype® (WO 01/79540). A decrease in susceptibility vis-à-vis the wild type virus correlates to an increased viral drug resistance, and hence reduced effectiveness of the drug.

The invention's methods are also useful for constructing a database that correlates HIV genotype to HIV phenotypic susceptibility to at least one test compound. Thus, in one embodiment, the HIV RNA sequence (e.g., from an HIV-infected subject) comprises at least one mutation relative to a reference HIV RNA sequence, and the database comprises a listing of the mutation. Such databases may be used to predict the drug susceptibility phenotype of a virus strain based on the genotypic results. The results of genotyping may be interpreted in conjunction with phenotyping and subjected to database interrogation, such as by virtual phenotyping (WO 01/79540).

In one embodiment of virtual phenotyping, the nucleotide sequence of HIV RNA may be used. In another embodiment, the genotypes are reported as amino acid changes at positions along the HIV gene products compared to a reference sequence, e.g., the wild-type HIV strain, HXB2 (SEQ ID NO:09 of FIG. 15). Analysis by VirtualPhenotype™ interpretational software (WO 01/79540) allows detection of mutational patterns in the database containing the genetic sequences of clinical isolates and linkage with the corresponding resistance profiles of the same isolates.

For example, in the process of virtual phenotyping, the genotype of a patient-derived HIV sequence may be correlated to the phenotypic response of the patient-derived HIV sequence. A report may be prepared including the EC₅₀ of the viral strain for one or more drugs, the sequence of the strain under investigation, and the biological cut-offs.

According to the methods described herein, a database may be constructed comprising genotypic and phenotypic data of HIV sequences, wherein the database further provides a correlation between genotypes and phenotypes, and wherein the correlation is indicative of efficacy of a given drug regimen (Van Baelen, WO 2008/090185).

8. Kits

The invention contemplates kits comprising (a) any one or more of the vectors disclosed herein (exemplified by, but not limited to, the yeast vectors pRECnfl-TRPΔ(p2-INT)/URA3-hRluc, and pRECnfl-TRPΔ(p2-INT)/URA3-hRluc (SEQ ID NO:08 of FIG. 23) (FIG. 10), and the eukaryotic vector pNL4-3-Δ(p24-VPR)-hRluc (SEQ ID NO:07 of FIG. 22), which is also referred to a pNL4-3-Δ(SphI-SalI)-hRluc in FIGS. 9 and 10), and (b) instructions for using the vectors.

The term “kit” is used in reference to a combination of reagents and other materials. It is contemplated that the kit may include reagents such as buffering agents, nucleic acid stabilizing reagents, protein stabilizing reagents, signal producing systems (e.g., fluorescence generating systems such as fluorescence resonance energy transfer (FRET) systems, radioactive isotopes, etc.), restriction enzymes, control proteins, control nucleic acid sequences, as well as testing containers (e.g., microtiter plates, etc.). It is not intended that the term “kit” be limited to a particular combination of reagents and/or other materials. In one embodiment, the kit further comprises instructions for using the reagents. The test kit may be packaged in any suitable manner, typically with the elements in a single container or various containers as necessary along with a sheet of instructions for carrying out the test. In some embodiments, the kits also preferably include a positive control sample. Kits may be produced in a variety of ways that are standard in the art. In some embodiments, the kits contain at least one reagent for amplifying a DNA sequence of interest, such as primers, enzymes, etc.

### EXPERIMENTAL

The following examples serve to illustrate certain embodiments and aspects of the present invention and are not to be construed as limiting the scope thereof.

### EXAMPLE 1

### Preliminary Experiments to Grow the Virus in MT-4 Cells

The complementation system based in the co-transfection of pRECnfl-AK-Δ(p2-Int)/URA3-hRluc+pCMV_cpltRU5gag into HEK 293T cells was used to construct recombinant viruses carrying HIV-1 p2-INT fragments from clinical samples (i.e., the p2/p7/p1/p6 region of gag and all of protease, reverse transcriptase, and integrase coding regions of poly, exemplified by SEQ ID NO:03 of FIG. 18. Unfortunately, we soon observed that we were not able to propagate in MT-4 cells all the viruses obtained after co-transfection of both plasmids into HEK 293T cells (Table 1). The data show a low success rate despite getting enough yeast colonies and having the correct plasmid transfected into HEK 293T cells.

TABLE 1
Virus production success rate (%)

| | | PCR | | | HEK | |
|---|---|---|---|---|---|---|
| | | product | Yeast | Bacteria | 293T | Virus |
| All (project | # | 95 | 93 | 93 | 93 | 76 |
| completed | Samples | | | | | |
| with verified | Success | 100% | 98% | 100% | 100% | 82% * |
| sequence) - | Rate | | | | | |
| (n = 95) | | | | | | |

* 80% cumulative

### EXAMPLE 2

Preliminary Experiment to Grow Virus—Growth in MT-4 Cells Took from 12 to 30 Days

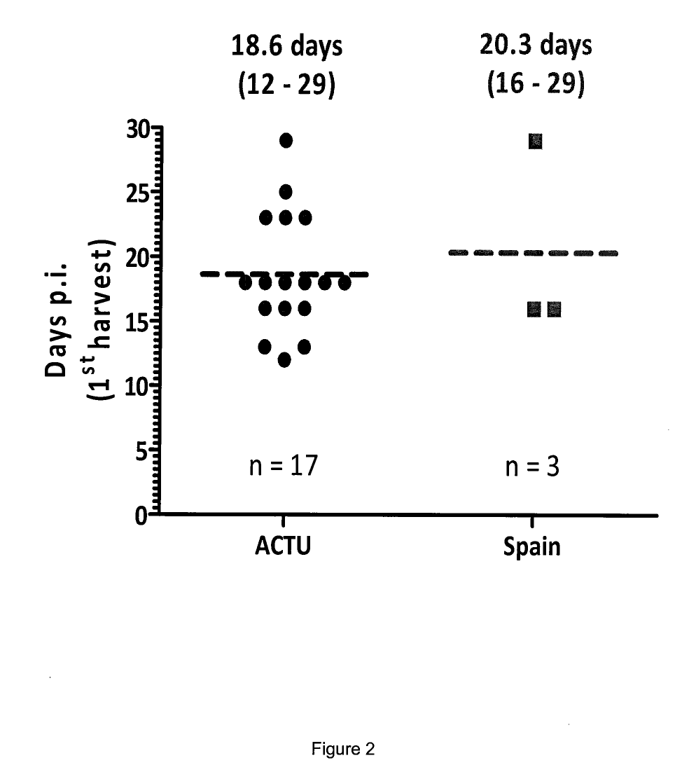

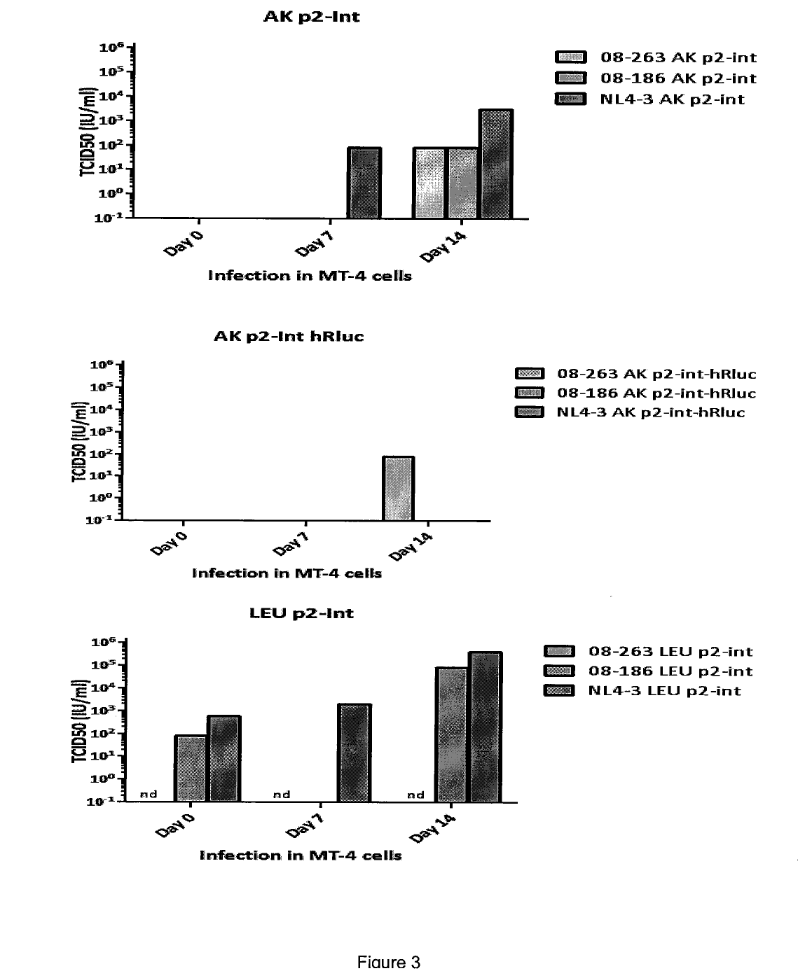

In addition to the problem obtaining 100% of the viruses most of them needed to be grown in MT-4 cells for a period of time ranging from 12 to 30 days to obtain enough virus titer to be used in drug susceptibility assays (FIG. 2).

### EXAMPLE 3

Preliminary Experiment to Grow Virus—Virus Grown in MT-4 Cells has a Viral Sequence that Does Not Match that from the Original Sample

In numerous occasions, and perhaps more critical than a long turn-around-time, propagating the recombinant virus in MT-4 cells led to the selection of variants that replicate more efficient in vitro. HIV replicates as a swarm of different viruses or quasispecies (1). Thus, it is common that a patient is infected with a myriad of viruses harboring different amino acids (mutations) in any given position of the HIV genome. Unfortunately, growing the virus in MT-4 cells led to the production and characterization of recombinant viruses with a different genotype than that observed in the original clinical sample (Table 2). The data show that virus grew in MT-4 cells but the viral sequence did not match that from the original sample.

TABLE 2
Changes in HIV genotype due to lengthy virus propagation in MT-4 cells

| | Genotype | Genotype (virus |
|---|---|---|
| Virus | (bacteria) | grown in MT-4 cells) |
| 08-188 | 92E/Q; 155N/H | 92E/Q 155N |
| 08-191 | 11D; 24G; 25E; 39C; 66T/I; 97A; | 11D; 24G; 25E; 39C; 66T; 97A; |
| | 101I; 112I; 119G; 122I; 125A; | 101I; 112I; 119G; 122I; 125A; |
| | 147G; 155N/H; 201I; 234V | 147G; 155N; 201I; 234V |
| 08-205 | 101I; 106A; 147G/S; 148Q/R; | 101I; 106A; 147G/S; 148Q/R; |
| | 155N/H; 193E; 201I; 206S 212G; | 155N; 193E; 201I; 206S; 212G; |
| | 230S/R; 232D/N; 256E; 288D/N | 230S; 232D/N; 256E; 288D |
| 08-219 | 31I; 42R/K; 66T/K; 85E/Q; 92E/Q; | 31I; 42R/K; 66T; 85E; 92E; |
| | 101I; 111K/R; 112V; 119S/R; 124N; | 101I; 111R; 112V; 119R; 124N; |
| | 125V; 135V; 155N/H; 201I; 206S; | 125V; 135V; 155N/H; 201I; |
| | 215N; 216Q/R; 253D/H; 256E | 206S; 215N ; 216Q; 253D; 256E |
| 08-246 | 17V; 31I; 51Y; 113V; 124N; | 17V; 31I; 51Y; 113V; 124N; |
| | 125A; 145S; 148R; 201I | 125A; 145S; 148Q; 201I |

Underlined amino acids were lost after propagating the virus in MT-4 cells

### EXAMPLE 4

Vector with the Renilla Gene Does Not Produce Recombinant Virus Efficiently:

At this point the data showed that the introduction of the renilla luciferase gene into the pRECnfl-AK-Δ(p2-Int)/URA3 may have been affecting the ability of the virus to replicate. Thus, we used three different samples, i.e., an antiretroviral naïve (08-263), a multidrug resistant strain (08-186), and a wild-type control (pNL4-3, exemplified by SEQ ID NO:06 of FIG. 21) to compare the virus production using three vectors expressing or not hRluc (Table 3).

TABLE 3
Virus production success rate

| | Samples | | |
|---|---|---|---|
| | | 08-186 | pNL4-3 |
| | 08-263 | (multidrug | (wt |
| Vectors | (ARV naive) | resistant) | control) |
| pRECnfl-AK-Δ(p2-Int)/URA3 | ✓ | ✓ | ✓ |
| pRECnfl-AK-Δ(p2-Int)/URA3- | ✓ | ✓ | ✓ |
| hRluc | | | |
| pRECnfl-LEU-Δ(p2-Int)/URA3 | not determined | ✓ | ✓ |

As observed in FIG. 3, viruses constructed using the pRECnfl-AK-Δ(p2-Int)/URA3 needed to be propagated longer than the viruses constructed using the original pRECnfl-LEU-(Δp2-Int)/URA3 to obtain a detectable titer (i.e., 10² to 10³ IU/ml). More important, only one virus was detected at day 14 when using the vector expressing the renilla luciferase gene (pRECnfl-AK-Δ(p2-Int)/URA3-hRluc).

In conclusion, the data showed that (i) trimming the original pRECnfl-LEU vector to create the pRECnfl-AK vector seem to have adversely affected the efficiency of the complementation system (i.e., co-transfection of the two plasmids into the HEK 293T cells) to generate viable virions and (ii) the introduction of the renilla luciferase gene into the pRECnfl-AK vector impaired the system even more.

### EXAMPLE 5

Construction of HIV-1 Tagged with Renilla or Firefly Luciferase Genes:

HIV-1 replication competent viruses were generated as luminescence variants expressing firefly (fluc2) or Renilla (hRluc) proteins in a HIV-1_{NL4-3} genotypic background as described (5). No viral gene was deleted or affected in this process. FIG. 4 summarizes the construction of these vectors.

Fluc2- and hRluc-tagged viruses showed similar replication kinetics and stability over multiple rounds of replication in U87.CD4.CCR5/CXCR4 cells, and were able to infect a variety of other CXCR4 and CCR5 expressing cells (i.e., MT-4, MT-2, HUT78, 174xCEM, PM1, GHOSTX4/R5, and TZM-bl) (FIG. 5). Briefly, to test the stability of the reporter genes, we infected MT-4 cells with either the recombinant pNL4-3 that expresses firefly (fluc2) or renilla (hRluc) genes and quantified viral replication (virus production) using a reverse transcriptase assay. Expression of the reporter gene in the cells was quantified using a luciferase assay. We monitored the cultures every 3 to 4 days for 42 days. A ratio of virus production/luciferase expression (cpm in the RT assay/RLU in the luciferase assay) provided data on whether the plasmids were “loosing” expression of the reporter gene with each passage, despite the fact that the virus continues to replicate.

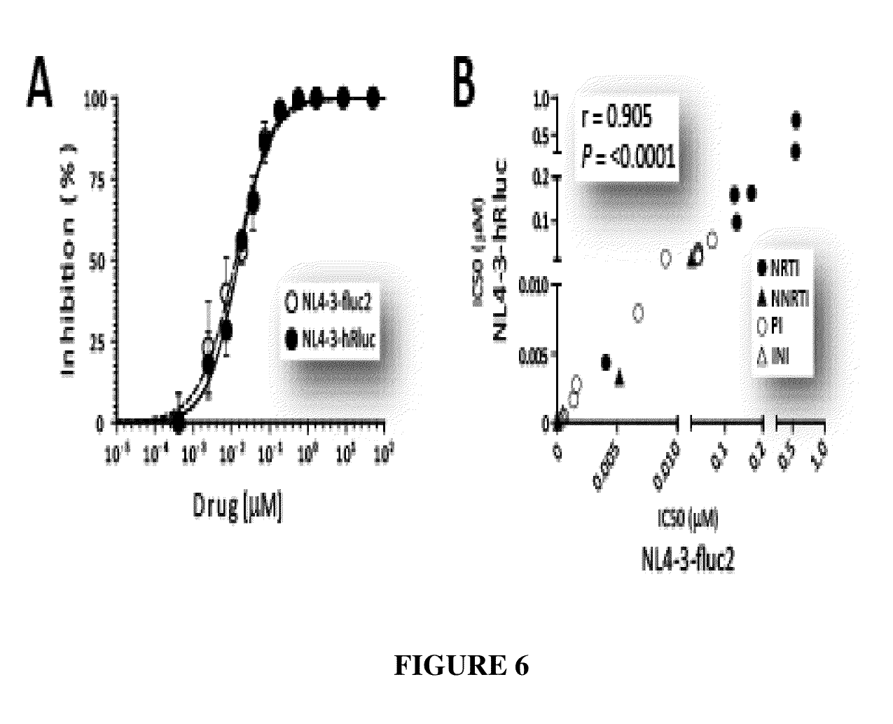

Furthermore, these viruses were successfully used in drug susceptibility (IC₅₀) determinations of different classes of antiretroviral drugs (i.e., protease, reverse transcriptase, and integrase inhibitors) (FIG. 6).

### EXAMPLE 6

Construction of a Single Exemplary Vector pNL4-3-Δ(SphI-SalI)-hRluc (also Referred to herein as pNL4-3-Δ(p24-VPR)-hRluc) Based on the HIV-1NL4-3 Background Lacking the p2/p7/p1/p6/PR/RT/INT-Coding Region, and Expressing the Renilla Luciferase Gene:

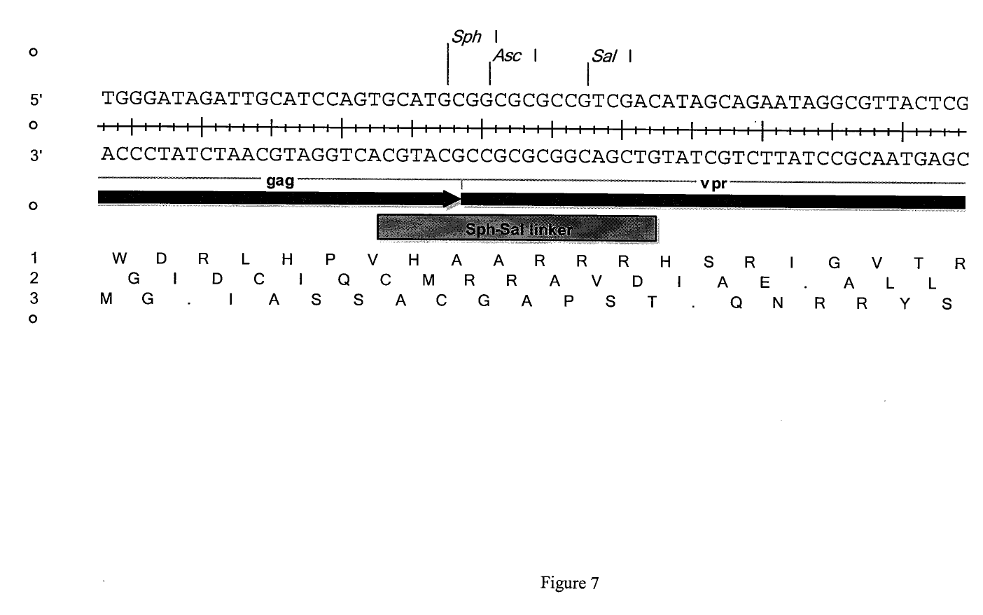

In order to create p2-Int recombinant viruses we replaced this HIV-1 region in the pNL4-3-hRluc vector with a non-HIV sequence that acts as a linker fragment. Briefly, a SphI-SalI linker was prepared by mixing 30 μg of forward primer 5′-TCCAGTGCATGCGGCGCGCCGTCGACATAGCA-3′ with 30 μg reverse primer 5′-TGCTATGTCGACGGCGCGCCGCATGCACTGGA-3′ (both from Invitrogen), heated for 1 min at 94° C., slowly cooled to 37° C. in a block heater and incubated for one hour. Annealed linker was double digested for 3 hours with SphI and SalI (New England Biolabs) at 37° C. and phosphorylated using T4 polynucleotide kinase (New England Biolabs) for 30 minutes at 37° C. followed by heat inactivation for 10 minutes at 65° C. The pNL4-3-hRluc vector was double digested with SphI and SalI at 37° C. and gel purified (E-Gel, Invitrogen) to remove the unwanted 4,333 by SphI-SalI fragment from the HIV-1_{NL4-3} strain. Twenty nanograms of this vector was then ligated at 16° C. with a range of vector:linker ratios (i.e., 1:1 to 1:20) using T4 ligase (New England Biolabs) for 16 hours. The ligase enzyme was heat inactivated for 10 minutes at 65° C. and one tenth of the ligation reaction was transformed by electroporation into electrocompetent Top 10 cells (Invitrogen). The 1:20 vector:linker ratio had the highest number of colonies and six colonies were analyzed. All six clones were positive (contained vector with the linker) as demonstrated by the digestion with the AscI enzyme (this restriction site was introduced with the linker, FIG. 7).

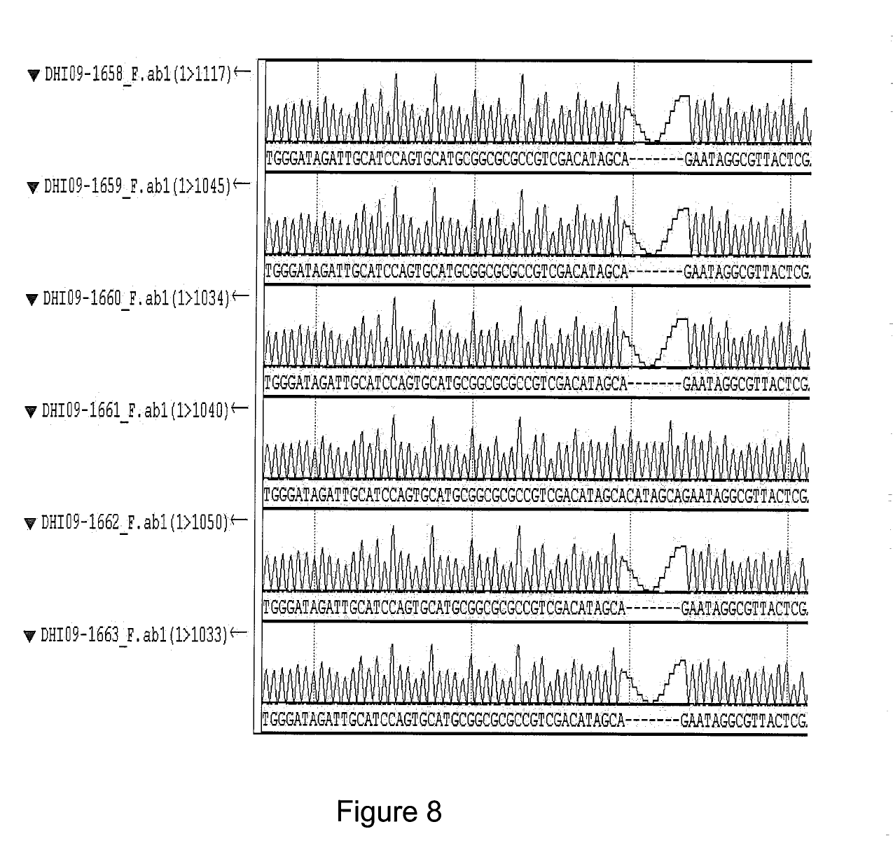

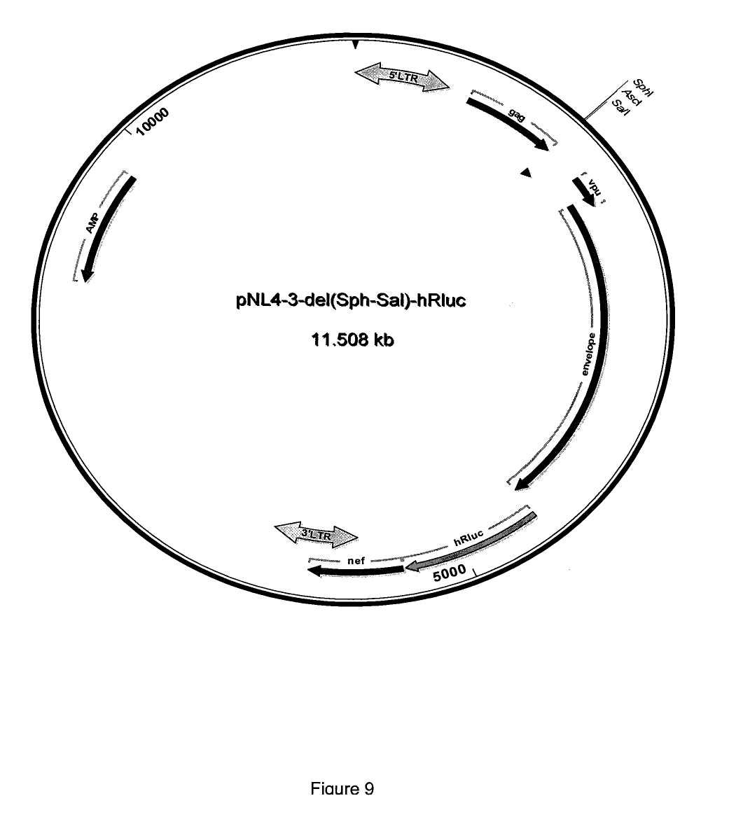

In addition, the sequence of all six clones was verified to corroborate the correct introduction of the linker into the pNL4-3-hRluc vector. Five out of the six clones contained the right form of the linker (FIG. 8). FIG. 9 depicts a schema of the invention's pNL4-3-Δ(SphI-SalI)-hRluc vector (also referred to herein as pNL4-3-Δ(p24-VPR)-hRluc).

### EXAMPLE 7

The pNL4-3-Δ(SphI-SalI)-hRluc Vector (also Referred to Herein as pNL4-3-Δ(p24-VPR)-hRluc) is Able to Produce High Titer Replication Competent p2-Int-Recombinant Virus Following Plasmid Transfection into HEK 293T Cells, Without Propagation in MT-4 Cells.

Different attempts to grow a p2-Int-recombinant virus obtained from a highly antiretroviral-experienced patient infected (08-188) with a multidrug resistant HIV-1 strain were unsuccessful, despite having enough plasmid DNA to transfect HEK 293T using the pRECnfl-AK-Δp2-Int or the pRECnfl-LEU-Δp2-Int by the complementation method, i.e., co-transfection of two vectors. For that reason, we selected the same clinical sample to test the functionality of the pNL4-3-Δ(SphI-SalI)-hRluc vector (also referred to herein as pNL4-3-Δ(p24-VPR)-hRluc) to produce high titer p2-Int-recombinant virus two days after transfection. FIG. 10 summarizes the process. Briefly, one ml of plasma was centrifuged at 20,000×g for 60 minutes at 4° C. After removal of 860 μl of supernatant the pellet was resuspended in the remaining 140 μl of supernatant and viral RNA was extracted using QIAamp Viral RNA Mini kit (Qiagen). The RNA was reverse-transcribed using AccuScript High Fidelity Reverse Transcriptase (Agilent) and the corresponding antisense external primer in 20 μl of reaction mixture containing 1 mM dNTPs, 10 mM DTT and 10 units of RNAse inhibitor. Viral cDNA was further amplified by two rounds of PCR using a set of external and nested primers. The external PCR was carried out in 50 μl reaction mixture containing 0.2 mM dNTPs, 3 mM MgCl₂ and 2.5 units of Pfu Turbo DNA Polymerase (Agilent). The nested PCR was carried out in 50 μl reaction mixture containing 0.2 mM dNTPs, 0.3 units of Pfu Turbo DNA Polymerase and 0.9 units of Taq Polymerase (Denville Scientific). The final PCR product spanning the 3′Gag (p2/p7/p1/p6), protease, reverse transcriptase and the integrase genes was cloned into the pRECnfl-TRPΔ(p2-INT)/URA3-hRluc vector (also referred to as pRECnfl-TRP-Δp2-Int-hRluc) (comprising a sequence exemplified by SEQ ID NO:08 of FIG. 23) using the yeast-based recombination/gap repair method as described (2). That is, the PCR product (˜2 μg) was transformed into yeast cells along with the pRECnfl-TRPΔ(p2-INT)/URA3-hRluc. Yeast colonies grew on CSM-TRP+5-FOA plates after 2 to 4 days carrying the pRECnfl-TRP-p2-INT vector with the foreign p2-INT gene. URA3 converts 5-FOA into a toxic anabolite such that yeast carrying the pRECnfl-TRP-Δp2-INT/URA3 vector cannot survive on the CSM-TRP+5-FOA plates. DNA vector was isolated from yeast colonies (yeast recombination/gap repair typically yields from 200 to 2,000 colonies) and transformed into Electrocomp TOP10 (Invitrogen). Ten to 20 μg of plasmid DNA was obtained using QIAprep Spin Miniprep Kit (Qiagen) from 10 ml of bacteria culture.

At this point, the SphI-SalI fragment was extracted from the pRECnfl-TRP-Δp2-INT/URA3 vector by double-digesting five micrograms of the vector with 30 units of SphI HF and 100 units of SalI HF for 4 hours at 37° C. The SphI-SalI fragment, containing the virus p2-Int region from the clinical sample, was purified (E-gel, Invitrogen). Ten micrograms of the pNL4-3-Δ(SphI-SalI)-hRluc vector (also referred to as pNL4-3-Δ(p24-VPR)-hRluc) containing the linker were (i) double digested with 60 units of SphI HF and 120 units of SalI HF for 3 hours at 37° C., (ii) dephosphorylated with 10 units of Antarctic phosphatase for 1 hour and (iii) PCR purified (Qiagen). The ligation reaction was performed at 16° C. for 3 hours with a 3:1 molar ratio of vector:fragment. One tenth of ligation product pNL4-3-Δ(p24-VPR)-hRluc was transformed by electroporation into Electrocomp Top10 cells (Invitrogen). All bacteria colonies were collected with 10 ml of LB medium with ampicillin and incubated overnight at 37° C. with shaking. Four micrograms of isolated plasmid DNA (Qiagen) were transfected into HEK 293T cells using GenDrill (BamaGen). Cell culture supernatant was harvested 48 hours post-transfection, clarified by centrifugation at 700×g, filtered through a 0.45 μm filter, aliquoted and stored at −80° C. for further use.

Tissue culture dose for 50% infectivity (TCID₅₀) was determined by infecting MT-4 cells in triplicate with serially diluted virus, calculated using the Reed and Muench method, and expressed as infectious units per milliliter (IU/ml). Finally, the phenotype (drug susceptibility) of the p2-Int recombinant 08-188 virus was quantified in MT-4 cells. For that, a mixture of the 08-188 (query) virus expressing hRluc and the NL4-3 (control) virus expressing fluc2 was used to infect MT-4 cells at a multiplicity of infection of 0.0025 IU/ml for one hour. HIV-infected cells were then grown for three days in triplicate with serial dilutions of twenty antiretroviral drugs at 37° C., 5% CO₂. Viral replication was quantified by measuring the expression of hRluc and fluc2 using Dual-Glo® Luciferase Assay System (Promega) in a Victor V multilabel reader (PerkinElmer). The 50% inhibitory concentration (IC₅₀) for each drug was calculated and graphs constructed using nonlinear regression analysis with GraphPad Prism version 5.02 for Windows (GraphPad Software, San Diego, Calif.) and the fold-resistance calculated based on the IC₅₀ values of the reference NL4-3-fluc2 virus.

### EXAMPLE 8

The Invention's Reporter-Tagged Viral Sequence Matches that from the Original Sample:

The 08-188 p2-Int recombinant constructed by transfecting the single pNL4-3-p2-Int₍₀₈₋₁₈₈₎-hRluc vector into HEK-293T cells had a high TCID₅₀ of 10^{6.3} IU/ml. More important, the amino acid sequence in the protease, RT, and integrase genes of the virus matched the original sequence obtained from the plasma sample, which then correlated with the drug susceptibility data (FIG. 11).

### EXAMPLE 9

The Invention's Vector with the Renilla Gene Produces Recombinant Virus Efficiently—Comparing the Production of Recombinant Virus Using the Art's Co-Transfection (Two Vectors) Method and the Invention's (Single Vector) Method:

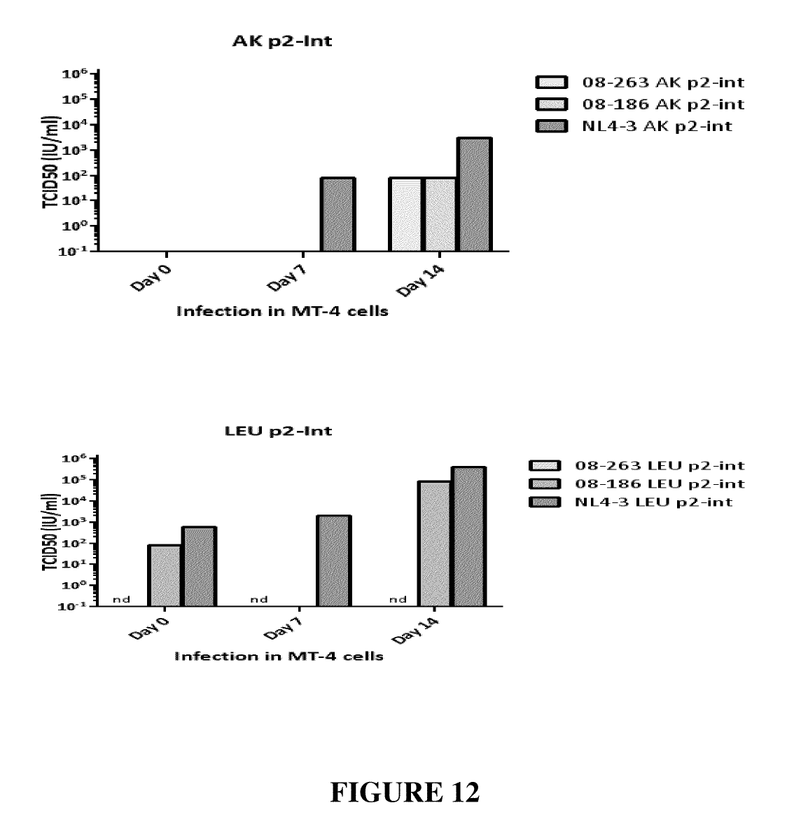

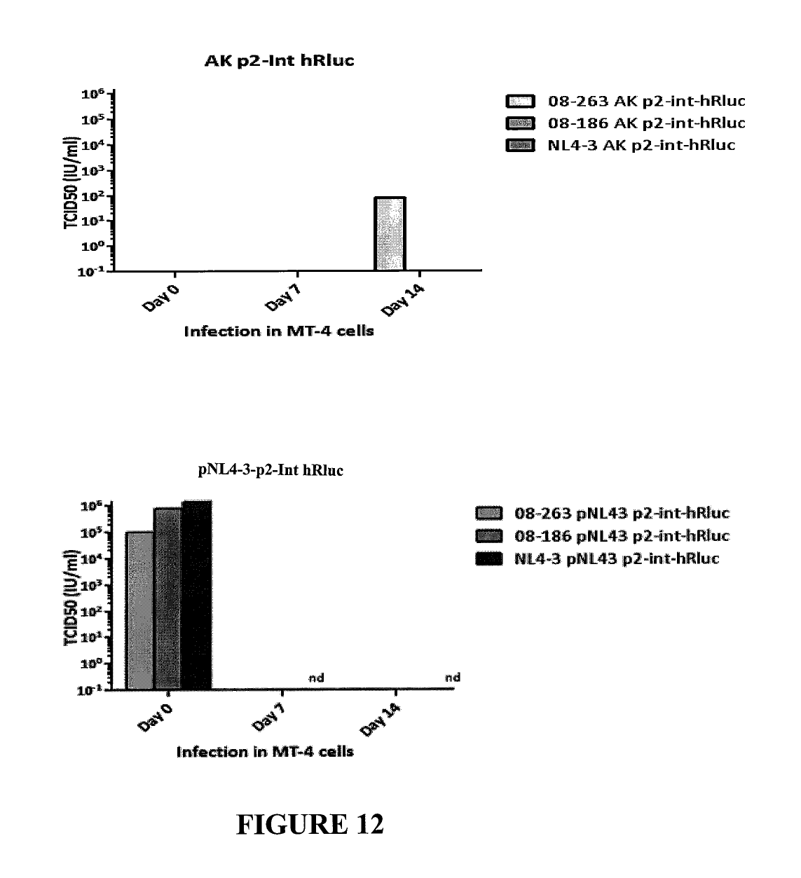

The results producing p2-Int recombinant viruses by transfecting HEK 293T cells with the invention's single vector were encouraging. For that reason, we tested the same three samples described in Table 2 and FIG. 3 with the invention's method (one vector) to compare the yield and time to produce recombinant virus with the art's complementation technology (two vectors). As observed in FIG. 12, the invention's single vector approach produced high titers (ranging from 10⁵ to 10^{6.3} IU/ml) of all three replication competent viruses two days after transfection (day 0) without propagation in MT-4 cells. In contrast, viruses produced with the pRECnfl vectors and complementation system had to be propagated for no less than two weeks to reach similar titers.

Importantly, the recombinant viruses obtained only 48 hours post-transfection also carry the renilla luciferase gene without a notable effect in viral replication.

In summary, using a single vector to transfect HEK 293T cells (i) reduces the time to obtain replication competent virus, (ii) increase the yield or titer of the virus without the need for propagation in HIV-susceptible cells, and (iii) allows the construction of recombinant viruses expressing reporter genes such as renilla or firefly luciferase. Table 4 compares some of the characteristics of the art's and the invention's approaches to construct recombinant viruses using the yeast-based cloning technology.

TABLE 4
Comparing he production of recombinant virus obtained
fro clinical samples using co-transection (two-vectors)
or and transfection (one vector) of HEK 293T cells.

| | | Invention's |
|---|---|---|
| | Art's Method | Exemplary Method |
| | (two plasmids) | (one plasmid) |
| Vectors | pCMV_cpltRU5gag | pRECnfl-TRP- |
| | pRECnfl-LEU- | Δ(p2-Int) |
| | Δp2-Int | pNL4- 3-Δp2- |
| | | Int-hRluc |
| Method to clone the patient- | Recombina- | Recombina- |
| derived viral PCR product | tion (yeast) | tion (yeast) |
| Sub-cloning of p2-Int | No | Yes |
| fragment into a vector | | |
| Producer cells | HEK 293T | HEK 293T |
| Virus propagation | Yes (MT-4 cells) | No |
| Time to get “enough” | 5-28 days | 2 days |
| virus to test | | |
| Typical TCID₅₀ (after trans- | <10³ IU/ml | 10⁵-10⁶ IU/ml |
| fection of HEK 293T cells) | | |
| Reporter gene | No | Yes (hRluc) |

### EXAMPLE 10

### HIV-1 Drug Susceptibility Assay

One of the goals for the construction of recombinant viruses tagged with reporter genes is to use them to quantify their phenotype with respect to susceptibility to a panel of antiretroviral drugs. As shown in FIG. 13, the invention's approach to construct p2-Int recombinant viruses reduces the total time to perform the HIV-1 phenotyping assay by 2 to 25 days, depending on the time needed to propagate the virus in the art's method.

### SOME REFERENCES

1. Domingo et al. 1997. Prog. Drug Res. 48:99-128.

2. Dudley et al. 2009. Biotechniques 46:458-467.

3. Hertogs et al. 1998. Antimicrob. Agents Chemother. 42:269-276.

4. Meyerhans et al. 1989. Cell 58:901-910.

5. Weber et al. 2006. J Virol Methods. 136:102-117.

All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiment, it should be understood that the invention as claimed should not be unduly limited to such specific embodiments. Indeed, various modifications of the described modes for carrying out the invention which are obvious to those skilled in the art and in fields related thereto are intended to be within the scope of the following claims.

We claim:

1. An in vitro method for producing a replication competent chimeric human immunodeficiency virus (HIV), comprising
a) providing
1) a first DNA sequence encoding an HIV RNA sequence,
2) a first restriction enzyme,
3) a second restriction enzyme,
4) a first yeast vector that lacks a second DNA sequence encoding HIV 5′ long terminal repeat (LTR), and that comprises a third DNA sequence encoding an HIV genome sequence, wherein said HIV genome sequence contains, in place of a sequence that corresponds to said first DNA sequence,
i) a restriction sequence which can be specifically cleaved by said first restriction enzyme, and
ii) a restriction sequence which can be specifically cleaved by said second restriction enzyme,
5) a second vector that comprises, in operable combination, a fourth DNA sequence encoding an HIV genome sequence, wherein said HIV genome sequence comprises a heterologous sequence in place of said sequence corresponding to said first DNA sequence, and wherein said heterologous sequence is flanked by
i) a restriction sequence which can be specifically cleaved by said first restriction enzyme, and
ii) a restriction sequence which can be specifically cleaved by said second restriction enzyme, and
6) a host cell,
b) introducing said first DNA sequence by homologous recombination into said first yeast vector to produce a second yeast vector that comprises said first DNA sequence flanked by
i) a restriction sequence which can be specifically cleaved by said first restriction enzyme, and
ii) a restriction sequence which can be specifically cleaved by said second restriction enzyme,
c) contacting said second yeast vector produced in step b) with said first restriction enzyme and with said second restriction enzyme, wherein said contacting produces a cleaved nucleotide sequence comprising said first DNA sequence,
d) introducing said cleaved nucleotide sequence produced in step c) into said second vector under conditions to substitute said heterologous sequence with said first DNA sequence, thereby producing a fourth vector that comprises, in operable combination, a fifth DNA sequence encoding an HIV genome sequence, wherein said HIV genome comprises said first DNA sequence in place of said sequence corresponding to said first DNA sequence, and
e) transfecting said fourth vector into said host cell to produce a replication competent chimeric HIV that comprises said first DNA sequence.

2. The method of claim 1, wherein said method comprises, prior to said transfecting of step e), transforming said fourth vector into a bacterial cell to produce a transformed bacterial cell.

3. The method of claim 1, further comprising purifying said fourth vector from said transformed bacterial cell.

4. The method of claim 1, further comprising f) contacting said replication competent chimeric HIV produced by step e) with a test compound.

5. The method of claim 4, further comprising g) determining phenotypic susceptibility of said HIV, that is produced in step e), to said test compound.

6. The method of claim 5, further comprising h) generating a database that comprises said phenotypic susceptibility of said HIV, that is produced by step e), to said test compound.

7. The method of claim 6, wherein said HIV RNA sequence comprises at least one mutation relative to a reference HIV RNA sequence, and wherein said database comprises a listing of said mutation.

8. The method of claim 1, wherein said steps from step a) to step d) do not include propagation of an HIV particle, that comprises said first DNA sequence, by a producer cell.

9. The method of claim 1, wherein said heterologous sequence of step a)5) is selected from the group consisting of a linker sequence and a lethal gene sequence.

10. The method of claim 1, wherein said first DNA sequence that is comprised in said replication competent chimeric HIV produced by step e), has 100% identity to said first DNA sequence in step a)1).

11. The method of claim 1, wherein said replication competent chimeric HIV that is produced by step e) is infectious of a cell that is susceptible to HIV.

12. The method of claim 1, wherein said HIV RNA sequence of step a)1) is from a sample obtained from an HIV-infected subject.

13. The method of claim 12, wherein said first DNA sequence is produced by reverse-transcribing and amplifying said HIV RNA sequence.

14. The method of claim 1, wherein said first yeast vector further comprises a heterologous reporter gene.

15. The method of claim 1, wherein said second vector further comprises a heterologous reporter gene.

16. The method of claim 1, wherein said first yeast vector of step a)4) comprises pRECnfl-TRPΔ(p2-INT)/URA3-hRluc having SEQ ID NO:08.

17. The method of claim 16, wherein said second vector of step 5) comprises pNL4-3-Δ(p24-VPR)-hRluc having SEQ ID NO:07.

18. A composition comprising a replication competent chimeric HIV produced by the method of claim 1.

19. A database produced by a method selected from the group consisting of the method of claim 6 and the method of claim 7.

20. A composition comprising a vector that
a) lacks a DNA sequence encoding HIV 5′ long terminal repeat (LTR), and
b) comprises an HIV genome sequence that contains, in place of a first DNA sequence encoding an HIV RNA sequence,
i) a restriction sequence which can be specifically cleaved by a first restriction enzyme, and
ii) a restriction sequence which can be specifically cleaved by a second restriction enzyme.

21. The composition of claim 20, wherein said vector further comprises a reporter gene.

22. The composition of claim 21, wherein said vector comprises pRECnfl-TRPΔ(p2-INT)/URA3-hRluc having SEQ ID NO:08.

23. The composition of claim 20, wherein said vector further comprises a second DNA sequence that corresponds to said first DNA sequence, wherein said second DNA sequence is from a HIV-infected subject.

24. A composition comprising a vector that comprises, in operable combination,
i) a DNA sequence encoding an HIV genome sequence containing a deletion of an HIV sequence, wherein the deleted HIV sequence is substituted by a heterologous sequence, and
ii) a reporter gene.

25. The composition of claim 24, wherein said vector further comprises iii) a first restriction sequence and a second restriction sequence that flank said heterologous sequence.

26. The composition of claim 25, wherein said vector comprises pNL4-3-Δ(p24-VPR)-hRluc having SEQ ID NO:07.

27. The composition of claim 24, wherein the deleted HIV sequence is substituted with a corresponding sequence from a HIV-infected subject.

28. A kit comprising (a) one or more composition selected from the group consisting of the composition of claim 20 and the composition of claim 24, and (b) instructions for using said composition.


SEQUENCE LISTING (13 sequences)

SEQ ID NO: 1 - DNA, 634 bp, Artificial sequence
  synthetic
tggaagggctaatttggtcccaaaaaagacaagagatccttgatctgtggatctaccacacacaaggctacttccctgattggcagaactacacaccagggccagggatcagatatccactgacctttggatggtgcttcaagttagtaccagttgaaccagagcaagtagaagaggccaatgaaggagagaacaacagcttgttacaccctatgagccagcatgggatggaggacccggagggagaagtattagtgtggaagtttgacagcctcctagcatttcgtcacatggcccgagagctgcatccggagtactacaaagactgctgacatcgagctttctacaagggactttccgctggggactttccagggaggtgtggcctgggcgggactggggagtggcgagccctcagatgctacatataagcagctgctttttgcctgtactgggtctctctggttagaccagatctgagcctgggagctctctggctaactagggaacccactgcttaagcctcaataaagcttgccttgagtgctcaaagtagtgtgtgcccgtctgttgtgtgactctggtaactagagatccctcagacccttttagtcagtgtggaaaatctctagca

SEQ ID NO: 2 - DNA, 1653 bp, Artificial sequence
  synthetic
atggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaa

SEQ ID NO: 3 - DNA, 3232 bp, Artificial sequence
  synthetic
ataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaagtatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagataaattggtcagtgctggaatcaggaaagtactatttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggat

SEQ ID NO: 4 - DNA, 936 bp, Artificial sequence
  synthetic
atggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggatatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagatgttcatcgagtccgaccctgggttcttttccaacgctattgtcgagggagctaagaagttccctaacaccgagttcgtgaaggtgaagggcctccacttcagccaggaggacgctccagatgaaatgggtaagtacatcaagagcttcgtggagcgcgtgctgaagaacgagcagtaa

SEQ ID NO: 5 - DNA, 4338 bp, Artificial sequence
  synthetic
cagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaagtatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagataaattggtcagtgctggaatcaggaaagtactatttttagatggaatagataaggcccaagaagaacatgagaaatatcacagtaattggagagcaatggctagtgattttaacctaccacctgtagtagcaaaagaaataccatttcagagtgataaatgtcagctaaaaggggaagccatgcatggacaagtagactgtgtagccagcttatggcagctagattgtacacatttagaaggaaaagttatcttggtagcaagcccaggaaccagtggatatatagaagcagaagtaattccagcagagacagggcaagaagttcatgtagtcctcttaaaattagcaggaagatggccagtaaaaacagtacatacagacacagcatactatttcaccagtactacagttaaggccgcctgttggtgggcggggatcaagaatggcagcagcattccctacaatccccaaagtcaaggagtaatagaatctatgaataaacaggaatttgaaattataggacaggtaagagatcaggctgaacatcttaagacagcagtagaattaaagatattcatccacaattttaaaagaaaaggggggattggggggtacagtgcacaaatggcagtagtagacataatagcaacagacatacaaactaaagaattacaaaaacaaggggaaagaattcaaaattttcgggtttattacagggacagcagagatccagtttggaaaattacaaaaaagctcctctggaaaggtgaaggggcagtagtaatacaagataatagtgacggaccagcaatgccaagaagaaaagcaaagatcatcagggattatggaaaacagatggcaataaaagtaggtgtggcaagtagacaggatgaggattaacacatggaaaagattagtaaaggtgatgatttatatttcaaggaaagctaaggactggttttatagacatcactatgaaagacaccatatgaaaataagttcagaagtacacatcccactaggggatgctaaattagtaattactaatccatggggtctgcatacaggagaaagagactggcatttgggtcagggagtctcaacaacatataggaaaaagagatatagcacacaagtagaccctgacctagcagaccaactcatagaatggcactattttgattgtttttcagaatctgctataagaaataccatattaggaattcatctgagtcctaggtgtgaatatcaagcaggacataacaaggtaggatctctacaacgtatagttctagcagcattaataaaaccaaaacagataaagccacctttgcctagtgtgtacttggcaacagaggacagatggaacaagccccagaagaccaagggccacagagggagtaggaaactgaatggacactagagcttttagaggaacttaagagtgaagctgttagacatccatacaatgtatggctccataacttaggacaacatatctatgaaacttacggggatacttttcctaggatggaagccataataagaattctgcaacaactgctgtttatccatttcagaattgggtg

SEQ ID NO: 6 - DNA, 14825 bp, Artificial sequence
  synthetic
tggaagggctaatttggtcccaaaaaagacaagagatccttgatctgtggatctaccacacacaaggctacttccctgattggcagaactacacaccagggccagggatcagatatccactgacctttggatggtgcttcaagttagtaccagttgaaccagagcaagtagaagaggccaatgaaggagagaacaacagcttgttacaccctatgagccagcatgggatggaggacccggagggagaagtattagtgtggaagtttgacagcctcctagcatttcgtcacatggcccgagagctgcatccggagtactacaaagactgctgacatcgagctttctacaagggactttccgctggggactttccagggaggtgtggcctgggcgggactggggagtggcgagccctcagatgctacatataagcagctgctttttgcctgtactgggtctctctggttagaccagatctgagcctgggagctctctggctaactagggaacccactgcttaagcctcaataaagcttgccttgagtgctcaaagtagtgtgtgcccgtctgttgtgtgactctggtaactagagatccctcagacccttttagtcagtgtggaaaatctctagcagtggcgcccgaacagggacttgaaagcgaaagtaaagccagaggagatctctcgacgcaggactcggcttgctgaagcgcgcacggcaagaggcgaggggcggcgactggtgagtacgccaaaaattttgactagcggaggctagaaggagagagatgggtgcgagagcgtcggtattaagcgggggagaattagataaatgggaaaaaattcggttaaggccagggggaaagaaacaatataaactaaaacatatagtatgggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacaccaaggaagccttagataagatagaggaagagcaaaacaaaagtaagaaaaaggcacagcaagcagcagctgacacaggaaacaacagccaggtcagccaaaattaccctatagtgcagaacctccaggggcaaatggtacatcaggccatatcacctagaactttaaatgcatgggtaaaagtagtagaagagaaggctttcagcccagaagtaatacccatgttttcagcattatcagaaggagccaccccacaagatttaaataccatgctaaacacagtggggggacatcaagcagccatgcaaatgttaaaagagaccatcaatgaggaagctgcagaatgggatagattgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaagtatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagataaattggtcagtgctggaatcaggaaagtactatttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaacacatggaaaagattagtaaaacaccatatgtatatttcaaggaaagctaaggactggttttatagacatcactatgaaagtactaatccaaaaataagttcagaagtacacatcccactaggggatgctaaattagtaataacaacatattggggtctgcatacaggagaaagagactggcatttgggtcagggagtctccatagaatggaggaaaaagagatatagcacacaagtagaccctgacctagcagaccaactaattcatctgcactattttgattgtttttcagaatctgctataagaaataccatattaggacgtatagttagtcctaggtgtgaatatcaagcaggacataacaaggtaggatctctacagtacttggcactagcagcattaataaaaccaaaacagataaagccacctttgcctagtgttaggaaactgacagaggacagatggaacaagccccagaagaccaagggccacagagggagccatacaatgaatggacactagagcttttagaggaacttaagagtgaagctgttagacattttcctaggatatggctccataacttaggacaacatatctatgaaacttacggggatacttgggcaggagtggaagccataataagaattctgcaacaactgctgtttatccatttcagaattgggtgtcgacatagcagaataggcgttactcgacagaggagagcaagaaatggagccagtagatcctagactagagccctggaagcatccaggaagtcagcctaaaactgcttgtaccaattgctattgtaaaaagtgttgctttcattgccaagtttgtttcatgacaaaagccttaggcatctcctatggcaggaagaagcggagacagcgacgaagagctcatcagaacagtcagactcatcaagcttctctatcaaagcagtaagtagtacatgtaatgcaacctataatagtagcaatagtagcattagtagtagcaataataatagcaatagttgtgtggtccatagtaatcatagaatataggaaaatattaagacaaagaaaaatagacaggttaattgatagactaatagaaagagcagaagacagtggcaatgagagtgaaggagaagtatcagcacttgtggagatgggggtggaaatggggcaccatgctccttgggatattgatgatctgtagtgctacagaaaaattgtgggtcacagtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaagtgcactgatttgaagaatgatactaataccaatagtagtagcgggagaatgataatggagaaaggagagataaaaaactgctctttcaatatcagcacaagcataagagataaggtgcagaaagaatatgcattcttttataaacttgatatagtaccaatagataataccagctataggttgataagttgtaacacctcagtcattacacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaatcaggccagtagtatcaactcaactgctgttaaatggcagtctagcagaagaagatgtagtaattagatctgccaatttcacagacaatgctaaaaccataatagtacagctgaacacatctgtagaaattaattgtacaagacccaacaacaatacaagaaaaagtatccgtatccagaggggaccagggagagcatttgttacaataggaaaaataggaaatatgagacaagcacattgtaacattagtagagcaaaatggaatgccactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcacactcccatgcagaataaaacaatttataaacatgtggcaggaagtaggaaaagcaatgtatgcccctcccatcagtggacaaattagatgttcatcaaatattactgggctgctattaacaagagatggtggtaataacaacaatgggtccgagatcttcagacctggaggaggcgatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctgatatagtgcagcagcagaacaatttgctgagggctattgaggcgcaacagcatctgttgcaactcacagtctggggcatcaaacagctccaggcaagaatcctggctgtggaaagatacctaaaggatcaacagctcctggggatttggggttgctctggaaaactcatttgcaccactgctgtgccttggaatgctagttggagtaataaatctctggaacagatttggaataacatgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaatcccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttaacttgctcaatgccacagccatagcagtagctgaggggacagatagggttatagaagtattacaagcagcttatagagctattcgccacatacctagaagaataagacagggcttggaaaggattttgctataagatgggtggcaagtggtcaaaaagtagtgtgattggatggcctgctgtaagggaaagaatgagacgagctgagccagcagcagatggggtgggagcagtatctcgagacctagaaaaacatggagcaatcacaagtagcaatacagcagctaacaatgctgcttgtgcctggctagaagcacaagaggaggaagaggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctgggcgggactggggagtggcgagccctcagatgctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcacccaggaggtagaggttgcagtgagccaagatcgcgccactgcattccagcctgggcaagaaaacaagactgtctaaaataataataataagttaagggtattaaatatatttatacatggaggtcataaaaatatatatatttgggctgggcgcagtggctcacacctgcgcccggccctttgggaggccgaggcaggtggatcacctgagtttgggagttccagaccagcctgaccaacatggagaaaccccttctctgtgtatttttagtagattttattttatgtgtattttattcacaggtatttctggaaaactgaaactgtttttcctctactctgataccacaagaatcatcagcacagaggaagacttctgtgatcaaatgtggtgggagagggaggttttcaccagcacatgagcagtcagttctgccgcagactcggcgggtgtccttcggttcagttccaacaccgcctgcctggagagaggtcagaccacagggtgagggctcagtccccaagacataaacacccaagacataaacacccaacaggtccaccccgcctgctgcccaggcagagccgattcaccaagacgggaattaggatagagaaagagtaagtcacacagagccggctgtgcgggagaacggagttctattatgactcaaatcagtctccccaagcattcggggatcagagtttttaaggataacttagtgtgtagggggccagtgagttggagatgaaagcgtagggagtcgaaggtgtccttttgcgccgagtcagttcctgggtgggggccacaagatcggatgagccagtttatcaatccgggggtgccagctgatccatggagtgcagggtctgcaaaatatctcaagcactgattgatcttaggttttacaatagtgatgttaccccaggaacaatttggggaaggtcagaatcttgtagcctgtagctgcatgactcctaaaccataatttcttttttgtttttttttttttatttttgagacagggtctcactctgtcacctaggctggagtgcagtggtgcaatcacagctcactgcagcctcaacgtcgtaagctcaagcgatcctcccacctcagcctgcctggtagctgagactacaagcgacgccccagttaatttttgtatttttggtagaggcagcgttttgccgtgtggccctggctggtctcgaactcctgggctcaagtgatccagcctcagcctcccaaagtgctgggacaaccggggccagtcactgcacctggccctaaaccataatttctaatcttttggctaatttgttagtcctacaaaggcagtctagtccccaggcaaaaagggggtttgtttcgggaaagggctgttactgtctttgtttcaaactataaactaagttcctcctaaacttagttcggcctacacccaggaatgaacaaggagagcttggaggttagaagcacgatggaattggttaggtcagatctctttcactgtctgagttataattttgcaatggtggttcaaagactgcccgcttctgacaccagtcgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccaggggaggcagagattgcagtaagctgagatcgcagcactgcactccagcctgggcgacagagtaagactctgtctcaaaaataaaataaataaatcaatcagatattccaatcttttcctttatttatttatttattttctattttggaaacacagtccttccttattccagaattacacatatattctatttttctttatatgctccagttttttttagaccttcacctgaaatgtgtgtatacaaaatctaggccagtccagcagagcctaaaggtaaaaaataaaataataaaaaataaataaaatctagctcactccttcacatcaaaatggagatacagctgttagcattaaataccaaataacccatcttgtcctcaataattttaagcgcctctctccaccacatctaactcctgtcaaaggcatgtgccccttccgggcgctctgctgtgctgccaaccaactggcatgtggactctgcagggtccctaactgccaagccccacagtgtgccctgaggctgccccttccttctagcggctgcccccactcggctttgctttccctagtttcagttacttgcgttcagccaaggtctgaaactaggtgcgcacagagcggtaagactgcgagagaaagagaccagctttacagggggtttatcacagtgcaccctgacagtcgtcagcctcacagggggtttatcacattgcaccctgacagtcgtcagcctcacagggggtttatcacagtgcacccttacaatcattccatttgattcacaatttttttagtctctactgtgcctaacttgtaagttaaatttgatcagaggtgtgttcccagaggggaaaacagtatatacagggttcagtactatcgcatttcaggcctccacctgggtcttggaatgtgtcccccgaggggtgatgactacctcagttggatctccacaggtcacagtgacacaagataaccaagacacctcccaaggctaccacaatgggccgccctccacgtgcacatggccggaggaactgccatgtcggaggtgcaagcacacctgcgcatcagagtccttggtgtggagggagggaccagcgcagcttccagccatccacctgatgaacagaacctagggaaagccccagttctacttacaccaggaaaggc

SEQ ID NO: 7 - DNA, 11454 bp, Artificial sequence
  synthetic
tggaagggctaatttggtcccaaaaaagacaagagatccttgatctgtggatctaccacacacaaggctacttccctgattggcagaactacacaccagggccagggatcagatatccactgacctttggatggtgcttcaagttagtaccagttgaaccagagcaagtagaagaggccaatgaaggagagaacaacagcttgttacaccctatgagccagcatgggatggaggacccggagggagaagtattagtgtggaagtttgacagcctcctagcatttcgtcacatggcccgagagctgcatccggagtactacaaagactgctgacatcgagctttctacaagggactttccgctggggactttccagggaggtgtggcctgggcgggactggggagtggcgagccctcagatgctacatataagcagctgctttttgcctgtactgggtctctctggttagaccagatctgagcctgggagctctctggctaactagggaacccactgcttaagcctcaataaagcttgccttgagtgctcaaagtagtgtgtgcccgtctgttgtgtgactctggtaactagagatccctcagacccttttagtcagtgtggaaaatctctagcagtggcgcccgaacagggacttgaaagcgaaagtaaagccagaggagatctctcgacgcaggactcggcttgctgaagcgcgcacggcaagaggcgaggggcggcgactggtgagtacgccaaaaattttgactagcggaggctagaaggagagagatgggtgcgagagcgtcggtattaagcgggggagaattagataaatgggaaaaaattcggttaaggccagggggaaagaaacaatataaactaaaacatatagtatgggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacaccaaggaagccttagataagatagaggaagagcaaaacaaaagtaagaaaaaggcacagcaagcagcagctgacacaggaaacaacagccaggtcagccaaaattaccctatagtgcagaacctccaggggcaaatggtacatcaggccatatcacctagaactttaaatgcatgggtaaaagtagtagaagagaaggctttcagcccagaagtaatacccatgttttcagcattatcagaaggagccaccccacaagatttaaataccatgctaaacacagtggggggacatcaagcagccatgcaaatgttaaaagagaccatcaatgaggaagctgcagaatgggatagattgcatccagtgcatgcggcgcgccgtcgacatagcagaataggcgttactcgacagaggagagcaagaaatggagccagtagatcctagactagagccctggaagcatccaggaagtcagcctaaaactgcttgtaccaattgctattgtaaaaagtgttgctttcattgccaagtttgtttcatgacaaaagccttaggcatctcctatggcaggaagaagcggagacagcgacgaagagctcatcagaacagtcagactcatcaagcttctctatcaaagcagtaagtagtacatgtaatgcaacctataatagtagcaatagtagcattagtagtagcaataataatagcaatagttgtgtggtccatagtaatcatagaatataggaaaatattaagacaaagaaaaatagacaggttaattgatagactaatagaaagagcagaagacagtggcaatgagagtgaaggagaagtatcagcacttgtggagatgggggtggaaatggggcaccatgctccttgggatattgatgatctgtagtgctacagaaaaattgtgggtcacagtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaagtgcactgatttgaagaatgatactaataccaatagtagtagcgggagaatgataatggagaaaggagagataaaaaactgctctttcaatatcagcacaagcataagagataaggtgcagaaagaatatgcattcttttataaacttgatatagtaccaatagataataccagctataggttgataagttgtaacacctcagtcattacacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaatcaggccagtagtatcaactcaactgctgttaaatggcagtctagcagaagaagatgtagtaattagatctgccaatttcacagacaatgctaaaaccataatagtacagctgaacacatctgtagaaattaattgtacaagacccaacaacaatacaagaaaaagtatccgtatccagaggggaccagggagagcatttgttacaataggaaaaataggaaatatgagacaagcacattgtaacattagtagagcaaaatggaatgccactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcacactcccatgcagaataaaacaatttataaacatgtggcaggaagtaggaaaagcaatgtatgcccctcccatcagtggacaaattagatgttcatcaaatattactgggctgctattaacaagagatggtggtaataacaacaatgggtccgagatcttcagacctggaggaggcgatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctgatatagtgcagcagcagaacaatttgctgagggctattgaggcgcaacagcatctgttgcaactcacagtctggggcatcaaacagctccaggcaagaatcctggctgtggaaagatacctaaaggatcaacagctcctggggatttggggttgctctggaaaactcatttgcaccactgctgtgccttggaatgctagttggagtaataaatctctggaacagatttggaataacatgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaatcccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttaacttgctcaatgccacagccatagcagtagctgaggggacagatagggttatagaagtattacaagcagcttatagagctattcgccacatacctagaagaataagacagggcttggaaaggattttgctataaaccggtcgccaccatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggatatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagatgttcatcgagtccgaccctgggttcttttccaacgctattgtcgagggagctaagaagttccctaacaccgagttcgtgaaggtgaagggcctccacttcagccaggaggacgctccagatgaaatgggtaagtacatcaagagcttcgtggagcgcgtgctgaagaacgagcagtaaagcggccgcatgggtggcaagtggtcaaaaagtagtgtgattggatggcctgctgtaagggaaagaatgagacgagctgagccagcagcagatggggtgggagcagtatctcgagacctagaaaaacatggagcaatcacaagtagcaatacagcagctaacaatgctgcttgtgcctggctagaagcacaagaggaggaagaggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctgggcgggactggggagtggcgagccctcagatgctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcacccaggaggtagaggttgcagtgagccaagatcgcgccactgcattccagcctgggcaagaaaacaagactgtctaaaataataataataagttaagggtattaaatatatttatacatggaggtcataaaaatatatatatttgggctgggcgcagtggctcacacctgcgcccggccctttgggaggccgaggcaggtggatcacctgagtttgggagttccagaccagcctgaccaacatggagaaaccccttctctgtgtatttttagtagattttattttatgtgtattttattcacaggtatttctggaaaactgaaactgtttttcctctactctgataccacaagaatcatcagcacagaggaagacttctgtgatcaaatgtggtgggagagggaggttttcaccagcacatgagcagtcagttctgccgcagactcggcgggtgtccttcggttcagttccaacaccgcctgcctggagagaggtcagaccacagggtgagggctcagtccccaagacataaacacccaagacataaacacccaacaggtccaccccgcctgctgcccaggcagagccgattcaccaagacgggaattaggatagagaaagagtaagtcacacagagccggctgtgcgggagaacggagttctattatgactcaaatcagtctccccaagcattcggggatcagagtttttaaggataacttagtgtgtagggggccagtgagttggagatgaaagcgtagggagtcgaaggtgtccttttgcgccgagtcagttcctgggtgggggccacaagatcggatgagccagtttatcaatccgggggtgccagctgatccatggagtgcagggtctgcaaaatatctcaagcactgattgatcttaggttttacaatagtgatgttaccccaggaacaatttggggaaggtcagaatcttgtagcctgtagctgcatgactcctaaaccataatttcttttttgtttttttttttttatttttgagacagggtctcactctgtcacctaggctggagtgcagtggtgcaatcacagctcactgcagcctcaacgtcgtaagctcaagcgatcctcccacctcagcctgcctggtagctgagactacaagcgacgccccagttaatttttgtatttttggtagaggcagcgttttgccgtgtggccctggctggtctcgaactcctgggctcaagtgatccagcctcagcctcccaaagtgctgggacaaccggggccagtcactgcacctggccctaaaccataatttctaatcttttggctaatttgttagtcctacaaaggcagtctagtccccaggcaaaaagggggtttgtttcgggaaagggctgttactgtctttgtttcaaactataaactaagttcctcctaaacttagttcggcctacacccaggaatgaacaaggagagcttggaggttagaagcacgatggaattggttaggtcagatctctttcactgtctgagttataattttgcaatggtggttcaaagactgcccgcttctgacaccagtcgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccaggggaggcagagattgcagtaagctgagatcgcagcactgcactccagcctgggcgacagagtaagactctgtctcaaaaataaaataaataaatcaatcagatattccaatcttttcctttatttatttatttattttctattttggaaacacagtccttccttattccagaattacacatatattctatttttctttatatgctccagttttttttagaccttcacctgaaatgtgtgtatacaaaatctaggccagtccagcagagcctaaaggtaaaaaataaaataataaaaaataaataaaatctagctcactccttcacatcaaaatggagatacagctgttagcattaaataccaaataacccatcttgtcctcaataattttaagcgcctctctccaccacatctaactcctgtcaaaggcatgtgccccttccgggcgctctgctgtgctgccaaccaactggcatgtggactctgcagggtccctaactgccaagccccacagtgtgccctgaggctgccccttccttctagcggctgcccccactcggctttgctttccctagtttcagttacttgcgttcagccaaggtctgaaactaggtgcgcacagagcggtaagactgcgagagaaagagaccagctttacagggggtttatcacagtgcaccctgacagtcgtcagcctcacagggggtttatcacattgcaccctgacagtcgtcagcctcacagggggtttatcacagtgcacccttacaatcattccatttgattcacaatttttttagtctctactgtgcctaacttgtaagttaaatttgatcagaggtgtgttcccagaggggaaaacagtatatacagggttcagtactatcgcatttcaggcctccacctgggtcttggaatgtgtcccccgaggggtgatgactacctcagttggatctccacaggtcacagtgacacaagataaccaagacacctcccaaggctaccacaatgggccgccctccacgtgcacatggccggaggaactgccatgtcggaggtgcaagcacacctgcgcatcagagtccttggtgtggagggagggaccagcgcagcttccagccatccacctgatgaacagaacctagggaaagccccagttctacttacaccaggaaaggc

SEQ ID NO: 8 - DNA, 14018 bp, Artificial sequence
  synthetic
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaacagtggcgcccgaacagggacttgaaagcgaaagtaaagccagaggagatctctcgacgcaggactcggcttgctgaagcgcgcacggcaagaggcgaggggcggcgactggtgagtacgccaaaaattttgactagcggaggctagaaggagagagatgggtgcgagagcgtcggtattaagcgggggagaattagataaatgggaaaaaattcggttaaggccagggggaaagaaacaatataaactaaaacatatagtatgggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacaccaaggaagccttagataagatagaggaagagcaaaacaaaagtaagaaaaaggcacagcaagcagcagctgacacaggaaacaacagccaggtcagccaaaattaccctatagtgcagaacctccaggggcaaatggtacatcaggccatatcacctagaactttaaatgcatgggtaaaagtagtagaagagaaggctttcagcccagaagtaatacccatgttttcagcattatcagaaggagccaccccacaagatttaaataccatgctaaacacagtggggggacatcaagcagccatgcaaatgttaaaagagaccatcaatgaggaagctgcagaatgggatagattgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccccgcggagattgtactgagagtgcaccataccaccttttcaattcatcattttttttttattcttttttttgatttcggtttccttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagcacatggaaaagattagtaaaacaccatatgtatatttcaaggaaagctaaggactggttttatagacatcactatgaaagtactaatccaaaaataagttcagaagtacacatcccactaggggatgctaaattagtaataacaacatattggggtctgcatacaggagaaagagactggcatttgggtcagggagtctccatagaatggaggaaaaagagatatagcacacaagtagaccctgacctagcagaccaactaattcatctgcactattttgattgtttttcagaatctgctataagaaataccatattaggacgtatagttagtcctaggtgtgaatatcaagcaggacataacaaggtaggatctctacagtacttggcactagcagcattaataaaaccaaaacagataaagccacctttgcccagtgttaggaaactgacagaggacagatggaacaagccccagaagaccaagggccacagagggagccatacaatgaatggacactagagcttttagaggaacttaagagtgaagctgttagacattttcctaggatatggctccataacttaggacaacatatctatgaaacttacggggatacttgggcaggagtggaagccataataagaattctgcaacaactgctgtttatccatttcagaattgggtgtcgacatagcagaataggcgttactcgacagaggagagcaagaaatggagccagtagatcctagactagagccctggaagcatccaggaagtcagcctaaaactgcttgtaccaattgctattgtaaaaagtgttgctttcattgccaagtttgtttcatgacaaaagccttaggcatctcctatggcaggaagaagcggagacagcgacgaagagctcatcagaacagtcagactcatcaagcttctctatcaaagcagtaagtagtacatgtaatgcaacctataatagtagcaatagtagcattagtagtagcaataataatagcaatagttgtgtggtccatagtaatcatagaatataggaaaatattaagacaaagaaaaatagacaggttaattgatagactaatagaaagagcagaagacagtggcaatgagagtgaaggagaagtatcagcacttgtggagatgggggtggaaatggggcaccatgctccttgggatattgatgatctgtagtgctacagaaaaattgtgggtcacagtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaagtgcactgatttgaagaatgatactaataccaatagtagtagcgggagaatgataatggagaaaggagagataaaaaactgctctttcaatatcagcacaagcataagagataaggtgcagaaagaatatgcattcttttataaacttgatatagtaccaatagataataccagctataggttgataagttgtaacacctcagtcattacacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaatcaggccagtagtatcaactcaactgctgttaaatggcagtctagcagaagaagatgtagtaattagatctgccaatttcacagacaatgctaaaaccataatagtacagctgaacacatctgtagaaattaattgtacaagacccaacaacaatacaagaaaaagtatccgtatccagaggggaccagggagagcatttgttacaataggaaaaataggaaatatgagacaagcacattgtaacattagtagagcaaaatggaatgccactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcacactcccatgcagaataaaacaatttataaacatgtggcaggaagtaggaaaagcaatgtatgcccctcccatcagtggacaaattagatgttcatcaaatattactgggctgctattaacaagagatggtggtaataacaacaatgggtccgagatcttcagacctggaggaggcgatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctgatatagtgcagcagcagaacaatttgctgagggctattgaggcgcaacagcatctgttgcaactcacagtctggggcatcaaacagctccaggcaagaatcctggctgtggaaagatacctaaaggatcaacagctcctggggatttggggttgctctggaaaactcatttgcaccactgctgtgccttggaatgctagttggagtaataaatctctggaacagatttggaataacatgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaatcccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttaacttgctcaatgccacagccatagcagtagctgaggggacagatagggttatagaagtattacaagcagcttatagagctattcgccacatacctagaagaataagacagggcttggaaaggattttgctataaaccggtcgccaccatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggatatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagatgttcatcgagtccgaccctgggttcttttccaacgctattgtcgagggagctaagaagttccctaacaccgagttcgtgaaggtgaagggcctccacttcagccaggaggacgctccagatgaaatgggtaagtacatcaagagcttcgtggagcgcgtgctgaagaacgagcagtaaagcggccgcatgggtggcaagtggtcaaaaagtagtgtgattggatggcctgctgtaagggaaagaatgagacgagctgagccagcagcagatggggtgggagcagtatctcgagacctagaaaaacatggagcaatcacaagtagcaatacagcagctaacaatgctgcttgtgcctggctagaagcacaagaggaggaagaggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctgggcgggactggggagtggcgagccctcagatgctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcctgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatagatcaacgacattactatatatataatataggaagcatttaatagaacagcatcgtaatatatgtgtactttgcagttatgacgccagatggcagtagtggaagatattctttattgaaaaatagcttgtcaccttacgtacaatcttgatccggagcttttctttttttgccgattaagaattaattcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgccgatctatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtatacgc

SEQ ID NO: 9 - DNA, 9719 bp, Artificial sequence
  synthetic
tggaagggctaattcactcccaacgaagacaagatatccttgatctgtggatctaccacacacaaggctacttccctgattagcagaactacacaccagggccagggatcagatatccactgacctttggatggtgctacaagctagtaccagttgagccagagaagttagaagaagccaacaaaggagagaacaccagcttgttacaccctgtgagcctgcatggaatggatgacccggagagagaagtgttagagtggaggtttgacagccgcctaggatttcatcacatggcccgagagctgcatccggagtacttcaagaactgctgacatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgdcttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggattaaataaaatagtaagaatgtatagccctaccagcattctggacataagacaaggadcaaaggaaccctttagagactatgtagaccggttctataaaactctaagagccgagcaagcttcacaggaggtaaaaaattggatgacaaaaaccttgttggtccaaaatgcgaacccagattgtaagactattttaaaagcattgggaccagcggctacactagaagaaatgatgacagcatgtcagggagtaggaggacccggccataaggcaagagttttggctgaagcaatgagccaagtaacaaattcagctaccataatgatgcagagaggcaattttaggaaccaaagaaagattgttaagtgtttcaattgtggcaaagaagggcacacagccagaaattgcagggcccctaggaaaaagggctgttggaaatgtggaaaggaaggacaccaaatgaaagattgtactgagagacaggctaattttttagggaagatctggccttcctacaagggaaggccagggaattttcttcagagcagaccagagccaacagccccaccagaagagagcttcaggtctggggtagagacaacaactccccctcagaagcaggagccgatagacaaggaactgtatcctttaacttccctcaggtcactctttggcaacgacccctcgtcacaataaagataggggggcaactaaaggaagctctattagatacaggagcagatgatacagtattagaagaaatgagtttgccaggaagatggaaaccaaaaatgatagggggaattggaggttttatcaaagtaagacagtatgatcagatactcatagaaatctgtggacataaagctataggtacagtattagtaggacctacacctgtcaacataattggaagaaatctgttgactcagattggttgcactttaaattttcccattagccctattgagactgtaccagtaaaattaaagccaggaatggatggcccaaaagttaaacaatggccattgacagaagaaaaaataaaagcattagtagaaatttgtacagagatggaaaaggaagggaaaatttcaaaaattgggcctgaaaatccatacaatactccagtatttgccataaagaaaaaagacagtactaaatggagaaaattagtagatttcagagaacttaataagagaactcaagacttctgggaagttcaattaggaataccacatcccgcagggttaaaaaagaaaaaatcagtaacagtactggatgtgggtgatgcatatttttcagttcccttagatgaagacttcaggaagtatactgcatttaccatacctagtataaacaatgagacaccagggattagatatcagtacaatgtgcttccacagggatggaaaggatcaccagcaatattccaaagtagcatgacaaaaatcttagagccttttagaaaacaaaatccagacatagttatctatcaatacatggatgatttgtatgtaggatctgacttagaaatagggcagcatagaacaaaaatagaggagctgagacaacatctgttgaggtggggacttaccacaccagacaaaaaacatcagaaagaacctccattcctttggatgggttatgaactccatcctgataaatggacagtacagcctatagtgctgccagaaaaagacagctggactgtcaatgacatacagaagttagtggggaaattgaattgggcaagtcagatttacccagggattaaagtaaggcaattatgtaaactccttagaggaaccaaagcactaacagaagtaataccactaacagaagaagcagagctagaactggcagaaaacagagagattctaaaagaaccagtacatggagtgtattatgacccatcaaaagacttaatagcagaaatacagaagcaggggcaaggccaatggacatatcaaatttatcaagagccatttaaaaatctgaaaacaggaaaatatgcaagaatgaggggtgcccacactaatgatgtaaaacaattaacagaggcagtgcaaaaaataaccacagaaagcatagtaatatggggaaagactcctaaatttaaactgcccatacaaaaggaaacatggaaaacatggtggacagagtattggcaagccacctggattcctgagtgggagtttgttaatacccctcccttagtgaaattatggtaccagttagagaaagaacccatagtaggagcagaaaccttctatgtagatggggcagctaacagggagactaaattaggaaaagcaggatatgttactaatagaggaagacaaaaaattgtcaccctaactgacacaacaaatcagaagactgagttacaagcaatttatctagctttgcaggattcgggattagaagtaaacatagtaacagactcacaatatgcattaggaatcattcaagcacaaccagatcaaagtgaatcagagttagtcaatcaaataatagagcagttaataaaaaaggaaaaggtctatctggcatgggtaccagcacacaaaggaattggaggaaatgaacaagtagataaattagtcagtgctggaatcaggaaagtactatttttagatggaatagataaggcccaagatgaacatgagaaatatcacagtaattggagagcaatggctagtgattttaacctgccacctgtagtagcagaagaaatagtagccagctgtgataaahgtcagctaaaaggagaagccatgcatggacaagtagactgtagtccaggaatatggcaactagattgtacacatttagaaggaaaagttatcctggtagcagttcatgtagccagtggatatatagaagcagaagttattccagcagaaacagggcaggaaacagcatattttcttttaaaattagcaggaagatggccagtaaaaacaatacatactgacaatggcagcaatttcaccggtgctacggttagggccgcctgttggtgggcgggaatcaagcaggaatttggaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccatatgtatgtttcagggaaagctaggggatggttttatagacatcactatgaaagccctcatccaagaataagttcagaagtacacatcccactaggggatgctagattggtaataacaacatattggggtctgcatacaggagaaagagactggcatttgggtcagggagtctccatagaatggaggaaaaagagatatagcacacaagtagaccctgaactagcagaccaactaattcatctgtattactttgactgtttttcagactctgctataagaaaggccttattaggacacatagttagccctaggtgtgaatatcaagcaggacataacaaggtaggatctctacaatacttggcactagcagcattaataacaccaaaaaagataaagccacctttgcctagtgttacgaaactgacagaggatagatggaacaagccccagaagaccaagggccacagagggagccacacaatgaatggacactagagcttttagaggagcttaagaatgaagctgttagacattttcctaggatttggctccatggcttagggcaacatatctatgaaacttatggggatacttgggcaggagtggaagccataataagaattctgcaacaactgctgtttatccattttcagaattgggtgtcgacatagcagaataggcgttactcgacagaggagagcaagaaatggagccagtagatcctagactagagccctggaagcatccaggaagtcagcctaaaactgcttgtaccaattgctattgtaaaaagtgttgctttcattgccaagtttgtttcataacaaaagccttaggcatctcctatggcaggaagaagcggagacagcgacgaagagctcatcagaacagtcagactcatcaagcttctctatcaaagcagtaagtagtacatgtaacgcaacctataccaatagtagcaatagtagcattagtagtagcaataataatagcaatagttgtgtggtccatagtaatcatagaatataggaaaatattaagacaaagaaaaatagacaggttaattgataggctaatggaaagagcagaagacagtggcaatgagagtgaaggagaaatatcagcacttgtggagatgggggtggagatggggcaccatgctccttgggatgttgatgatctgtagtgctacagaaaaattgtgggtcacagtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaagtgcactgatttgaagaatgatactaataccaatagtagtagcgggagaatgataatggagaaaggagagataaaaaactgctctttcaatatcagcacaagcataagaggtaaggtgcagaaagaatatgcatttttttataaacttgatataataccaatagataatgatactaccagctataagttgacaagttgtaacacctcagtcattacacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaattaggccagtagtatcaactcaactgctgttaaatggcagtctagcagaagaagaggtagtaattagatctgtcaatttcacggacaatgctaaaaccataatagtacagctgaacacatctgtagaaattaattgtacaagacccaacaacaatacaagaaaaagaatccgtatccagagaggaccagggagagcatttgttacaataggaaaaataggaaatatgagacaagcacattgtaacattagtagagcaaaatggaataacactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcaccctcccatgcagaataaaacaaattataaacatgtggcagaaagtaggaaaagcaatgtatgcccctcccatcagtggacaaattagatgttcatcaaatattacagggctgctattaacaagagatggtggtaatagcaacaatgagtccgagatcttcagacgtggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggamtcaggaactaaagaatagtgctgttagcttgctcaatgccacagccatagcagtagctgaggggacagatagggttatagaagtagtacaaggagcttgtagagctattcgccacatacctagaagaataagacagggcttggaaaggattttgctataagatgggtggcaagtggtcaaaaagtagtgtgattggatggcctactgtaagggaaagaatgagacgagctgagccagcagcagatagggtgggagcagcatctcgagacctggaaaaacatggagcaatcacaagtagcaatacagcagctaccaatgctgcttgtgcgtggctagaagcacaagaggaggaggaggtgggttttccagtcacacctcaggtacctttaagaccaatgacttacaaggcagttgtagatcttagccactttttaaaagaaaaggggggactggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataagatagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 10 - DNA, 6 bp, Artificial sequence
  synthetic
gcatgc

SEQ ID NO: 11 - DNA, 6 bp, Artificial sequence
  synthetic
gtcgac

SEQ ID NO: 12 - DNA, 8 bp, Artificial sequence
  synthetic
ggcgcgcc

SEQ ID NO: 13 - DNA, 20 bp, Artificial sequence
  synthetic
gcatgcggcgcgccgtcgac